(19) United States
(12) Patent Application Publication
(10) Pub. No.: US 2022/0211810 A1
(43) Pub. Date: Jul. 7, 2022

(54) Method of Treatment

(71) Applicant: Volution Immuno Pharmaceuticals SA, Geneva (CH)

(72) Inventors: Wynne H. Weston-Davies, Caine (GB); Virginia Calder, London (GB)

(73) Assignee: Volution Immuno Pharmaceuticals SA, Geneva (CH)

(21) Appl. No.: 17/604,509

(22) PCT Filed: Mar. 4, 2020
(86) PCT No.: PCT/EP2020/055741

§ 371 Date: Oct. 18, 2021

(30) Foreign Application Priority Data
Apr. 25, 2019 (GB) 1905810.6
Jan. 22, 2020 (GB) 2000955.1

(51) Int. Cl. A61K 38/17; A61P 27/02; A61K 45/06

(52) U.S. Cl. CPC A61K 38/1767; A61K 38/17; A61K 45/06; A61P 27/02

(57) ABSTRACT

The present invention relates to methods of treating or preventing proliferative retinal disease.

### FIELD OF THE INVENTION

The present invention relates to methods of treating and preventing proliferative retinal diseases.

All documents mentioned in the text and listed at the end of this description are incorporated herein by reference.

### BACKGROUND TO THE INVENTION

Complement

The complement system is an essential part of the body's natural defence mechanism against foreign invasion and is also involved in the inflammatory process. More than 30 proteins in serum and at the cell surface are involved in the functioning and regulation of the complement system. Recently, it has become apparent that, as well as the approximately 35 known components of the complement system, which may be associated with both beneficial and pathological processes, the complement system itself interacts with at least 85 biological pathways with functions as diverse as angiogenesis, platelet activation and haemostasis, glucose metabolism and spermatogenesis.

The complement system is activated by the presence of materials that are recognised by the immune system as non-self. Three activation pathways exist: (1) the classical pathway which is activated by IgM and IgG complexes or by recognition of carbohydrates; (2) the alternative pathway which is activated by non-self surfaces (lacking specific regulatory molecules) and by bacterial endotoxins; and (3) the lectin pathway which is activated by binding of mannan-binding lectin (MBL) to mannose residues on the surface of a pathogen. The three pathways comprise parallel cascades of events that result in the production of complement activation through the formation of similar C3¹ and C5 convertases on cell surfaces, resulting in the release of acute mediators of inflammation (C3a and C5a) and the formation of the membrane attack complex (MAC). The parallel cascades involved in the classical (here defined as classical via C1q and lectin via MBL) and alternative pathways are shown in FIG. 1. ¹ It is conventional to refer to the components of the complement pathway by the letter “C” followed by a number, such as “3”, such that “C3” refers to complement protein C3. Some of these components are cleaved during activation of the complement system and the cleavage products are given lower case letters after the number. Thus, C5 is cleaved into fragments which are conventionally labelled C5a and C5b. The complement proteins do not necessarily act in their number order and so the number does not necessarily give any indication of the order of action. This naming convention is used in this application.

The classical complement pathway, the alternative complement pathway and the lectin complement pathway are herein collectively referred to as the complement pathways. C5b initiates the ‘late’ or ‘terminal’ events of complement activation. These comprise a sequence of polymerization reactions in which the terminal complement components interact to form the MAC, which creates a pore in the cell membranes of some pathogens which can lead to their death or activates the body's own cells without causing lysis. The terminal complement components include C5b (which initiates assembly of the membrane attack system), C6, C7, C8 and C9.

LTB4

Leukotriene B4 (LTB4) is the most powerful chemotactic and chemokinetic eicosanoid described and promotes adhesion of neutrophils to the vascular endothelium via upregulation of integrins [1]. It is also a complete secretagogue for neutrophils, induces their aggregation and increases microvascular permeability. LTB4 recruits and activates natural killer cells, monocytes and eosinophils. It increases superoxide radical formation [2] and modulates gene expression including the production of a number of proinflammatory cytokines and mediators which may augment and prolong tissue inflammation [3,4]. LTB4 also has roles in the induction and management of adaptive immune responses. For example, regulation of dendritic cell trafficking to draining lymph nodes [5,6], Th2 cytokine IL-13 production from lung T cells [7], recruitment of antigen-specific effector CD8+ T cells [8] and activation and proliferation of human B lymphocytes [9].

LTB4 and the hydroxyeicosanoids mediate their effects though the BLT1 and BLT2 G-protein coupled receptors [10,11]. Human BLT1 is a high affinity receptor (Kd 0.39-1.5 nM; [12]) specific for LTB4 with only 20-hydroxy LTB4 and 12-epi LTB4 able to displace LTB4 in competitive binding studies [13]. Human BLT2 has a 20-fold lower affinity (Kd 23 nM) for LTB4 than BLT1 and is activated by binding a broader range of eicosanoids including 12-epi LTB4, 20-hydroxy LTB4, 12(S)- and 15(S)-HETE and 12(S)- and 15(S)-HPETE [13]. Human BLT2 has 45.2 and 44.6% amino acid identity with human and mouse BLT1, while human and mouse BLT2 have 92.7% identity [11].

Human BLT1 is mainly expressed on the surface of leukocytes, though it has recently been described in endothelial cells and vascular smooth muscle cells. Human BLT2 is expressed in a broader range of tissue and cell types. A number of specific antagonists of BLT1 and BLT2 have been described which inhibit activation, extravasation and apoptosis of human neutrophils [14 ] and reduce symptoms caused by neutrophil infiltration in mouse models of inflammatory arthritis [15] and renal ischaemia reperfusion [16]. Increasing numbers of studies indicate that both BLT1 and BLT2 can mediate pathological effects through LTB4 and hydroxyeicosanoids [17], although BLT1 certainly has a dominant role in some pathologies such as collagen induced arthritis in mice [18]. BLT1−/− deficient mice have also highlighted the importance of BLT1 in directing neutrophil migration in inflammatory responses. In particular, a 5LO deficient mouse strain was used to show autocrine activation of BLT1 on neutrophils is needed for their recruitment into arthritic joints [19].

A number of marketed drugs target the eicosanoids. These include the glucocorticoids which modulate phopholipase A2 (PLA2) and thereby inhibit release of the eicosanoid precursor arachidonic acid (AA) [20]. Non-steroidal antiinflammatory drugs (NSAID) and other COX2 inhibitors which prevent synthesis of the prostaglandins and thromboxanes [21]. There are also a number of leukotriene (LK) modifiers which either inhibit the 5-LOX enzyme required for LTB4 synthesis and other leukotrienes (Zileuton; [22]), or antagonise the CysLT1 receptor that mediates the effects of cysteinyl leukotrienes (Zafirlukast and Montelukast) [23]. The LK modifiers are orally available and have been approved by the FDA for use in the treatment of e.g. asthma. No drug that acts specifically on LTB4 or its receptors has yet reached the market.

Eye Conditions

Background

The invention concerns the treatment of proliferative retinal diseases, which are retinal conditions that involve the formation of blood vessels on the retina. For example, blood vessels can be produced in response to reduced blood supply caused by retinal ischaemia. This neovasculation occurs in general in response to the growth hormone Vascular Endothelial Cell Growth Factor (VEGF), which stimulates the production of new blood vessels on the optic disc or on the retinal surface. However, these new blood vessels are particularly weak, prone to leaking and can easily rupture resulting in haemorrhage and severe visual loss.

Such diseases include autoimmune uveitis, infective uveitis, wet age-related macular degeneration (AMD) (choroidal neovascularisation), dry age-related macular degeneration (geographic atrophy), diabetic retinopathy, diabetic macular oedema, optic neuritis (e.g. glaucoma associated optic neuritis), retinal vein occlusion and retinopathy of prematurity. Also included are Stargardt disease and polypoidal choroidal vasculopathy. Of particular interest is uveitis.

Current treatments for certain of these conditions exist. For example in noninfectious uveitis, treatment is focused on control of the eye inflammation with anti-inflammatory medications, e.g. steroids (e.g. corticosteroids) given as eyedrops, or injected in or around the eye, orally (by mouth), or intravenously, or immunomodulatory therapy (IMT) drugs (e.g. methotrexate, azathioprine, and mycophenolate), biologic response modifier (BRM) drugs (e.g. anti-TNFalpha agents, which may be antibodies or fragments thereof that bind TNFalpha, such as infliximab or adalimumab). For wet AMD, diabetic macular odema, retinal vein occlusion, and diabetic retinopathy current treatments include anti-VEGF treatment. These include anti VEGF-A antibodies or fragments thereof (such as bevacizumab (Avastin), ranibizumab (Lucentis)), anti-VEGF apatmers (such as pegaptanib (Macugen)), and other VEGF antagonists such as aflibercept (Eylea) a recombinant fusion protein consisting of vascular endothelial growth factor (VEGF)-binding portions from the extracellular domains of human VEGF receptors 1 and 2, that are fused to the Fc portion of the human IgG1 immunoglobulin)). Such anti VEGF medicines may be injected intravitreally approximately every 1 to 2 months. These treatments have also been proposed for retinopathy of prematurity.

Proliferative retinal diseases remain an unmet clinical need.

Complement Inhibitors

WO 2004/106369 (Evolutec Limited [24]) relates to complement inhibitors. A particular subset of the disclosed complement inhibitors are directed at C5 and prevent C5 being cleaved into C5a and C5b by any of the complement activation pathways. A particular example of such an inhibitor of C5 cleavage is a protein produced by ticks of the species Ornithdoros moubata, which in mature form is a protein consisting of amino acids 19 to 168 of the amino acid sequence shown in FIG. 4 of WO 2004/106369. In WO 2004/106369, this protein is known by the names “rVA576”, “EV576” and “OmCI protein” and has more recently been known as “Coversin” [25]. This protein is referred to herein as “nomacopan” which is the INN for the protein.

In the tick, nomacopan is expressed as a pre-protein having a leader sequence comprising amino acids 1 to 18 of the amino acid sequence shown in FIG. 4 of WO 2004/106369 at the N-terminal end of the mature nomacopan protein. The leader sequence is cleaved off after expression. The mature protein has the sequence consisting of amino acids 19 to 168 of the amino acid sequence shown in FIG. 4 of WO 2004/106369 and FIG. 2 of the present application.

Nomacopan also has the ability to inhibit leukotriene B4 (LTB4) activity. The ability to bind LTB4 may be demonstrated by standard in vitro assays known in the art, for example by means of a competitive ELISA between nomacopan and an anti-LTB4 antibody competing for binding to labelled LTB4, by isothermal titration calorimetry or by fluorescence titration. There are a number of further patent applications, such as WO 2007/028968, WO 2008/029167, WO 2008/029169, WO 2011/083317 and WO 2016/198133, which relate to the use of nomacopan or functional equivalents thereof in various applications. There is no experimental evidence in these applications that confirms the efficacy of nomacopan or any functional equivalent thereof in the treatment of proliferative retinal disease.

In work leading to the present invention, the inventors have shown that the LTB4 receptor and the C5a receptor are co-located on cells within the retinas of mice used in an experimental autoimmune uveitis module, where the disease was induced by injection of retinal binding protein (RBP3). At least some of these cells are believed to be M2 macrophages which are known to migrate to areas of retinal damage where they release VEGF in response to LTB4 stimulation. Furthermore, when injected intravitreally, the molecule nomacopan (which binds LTB4 and which also inhibits the complement pathway by binding to C5), as discussed above, and long acting versions of both nomacopan and LTB4-specific-nomacopan (or “L-nomacopan”, which binds LTB4 but which does not inhibit the complement pathway by binding to C5), referred to herein as PAS-nomacopan and PAS-L-Nomacopan, have been shown to improve clinical scores and composite histological scores in the experimental autoimmune uveitis (EAU) model. These molecules also reduced the population of Th17 cells (and IL-17). Th17 subsets have been detected in human uveitis and mediate disease in EAU. Nomacopan, L-nomacopan and long acting nomacopan, when administered topically, also had an effect on clinical scores in these mice, which is considered to be surprising as it was not believed that this molecule could penetrate the cornea.

The inventors have also shown that, in a separate mouse model of EAU, induction with RBP3 (also known as IRBP) results in a significant increase in VEGF levels in the retinal tissue. Intravitreal injection of nomacopan-type proteins results in a reduction of VEGF levels, as does intravitreal injection an anti-VEGF antibody (Example 4 and FIG. 7A). As nomacopan and its variants are not known to have any direct effect on VEGF, it is suggested that this is an indirect effect through at least the inhibition of LTB4 activation of the M2 macrophages referred to above, which are thought to be the principal source of VEGF in this model. The scavenging of LTB4 from neutrophils by nomacopan-type proteins is also thought to also have the effect of reducing the retinal to peripheral blood LTB4 concentration gradient thereby reducing inflammatory cell trafficking of Th17 cells (as shown in example 3) and macrophages.

Nomacopan has the ability to inhibit LTB4 and is therefore particularly advantageous in the prevention and treatment of proliferative retinal diseases, either alone or in combination with other treatments. It can furthermore inhibit the complement pathway (by inhibiting C5), which has certain advantages, e.g. where the complement pathway is involved in the disease pathology. Modified versions of nomacopan which inhibit LTB4 but which do not inhibit C5 may also be useful, and in particular in instances where it may be undesirable to inhibit the complement pathway.

Nomacopan has previously been disclosed as a potential treatment for eye surface conditions (e.g. atopic keratoconjunctivis, by administering the protein to the surface of the eye, see e.g. WO2018193120) but it was not previously known that the molecule could penetrate the cornea and thus be applied topically for the treatment of eye conditions that are not eye surface conditions, nor was it previously known that the molecule could be effective when administered directly into the eye. For example, even when administered directly into the eye, therapeutic molecules may not be able to reach the location at which they need to act to be effective, as various barriers (such as the inner limiting membrane and Bruch's membrane are present). Indeed, various previous complement inhibitor molecules have previously been tested for eye conditions but have not been found to be effective (see e.g. [26]).

Prior to the present work, it was not therefore known that nomacopan-type proteins could be used to treat proliferative retinal diseases, either by topical administration or by introduction directly into the eye. Furthermore, it was not known that nomacopan-type proteins could be used to reduce VEGF levels in retinal tissue.

### SUMMARY OF THE INVENTION

Nomacopan-type proteins have now been shown to reduce the clinical score in an experimental autoimmune uveitis mouse model, when administered topically or by introduction directly into the eye. This demonstrates the ability of such proteins to be of use in treating uveitis, as well as supporting the use of such proteins for the treatment of proliferative retinal diseases, either alone or in combination with other treatments. Nomacopan furthermore has the ability to inhibit both the complement pathway (by inhibiting C5) and also LTB4 and is therefore particularly advantageous in the prevention and treatment of proliferative retinal diseases where there is also a complement component, either alone or in combination with other treatments. The dual functions of the protein can furthermore be manipulated such that modified versions thereof that bind LTB4 but which do not bind to C5 (e.g. L-nomacopan) can be particularly advantageous in the prevention and treatment of proliferative retinal diseases where there is not a complement component, and/or where inhibiting the complement system might be disadvantageous. Again this can be either alone or in combination with other treatments.

In Example 1, topical administration of nomacopan-type proteins reduces clinical score in experimental autoimmune uveitis in a mouse model. In subsequent examples intravitreal administration of nomacopan-type proteins reduces clinical score in experimental autoimmune uveitis in a mouse model

The present inventors have therefore demonstrated that administration of the tick protein Nomacopan (also referred to as EV576 and OmCI in the art and herein [24], and previously referred to as “Coversin”) and variants thereof can be used to treat or prevent autoimmune uveitis and other proliferative retinal diseases.

The invention therefore provides a method of treating or preventing a proliferative retinal disease, which comprises administering a therapeutically or prophylactically effective amount of an agent which is a protein comprising amino acids 19 to 168 of the amino acid sequence in FIG. 2 (SEQ ID NO: 2) or a functional equivalent of this protein.

The invention also provides an agent which is a protein comprising amino acids 19 to 168 of the amino acid sequence in FIG. 2 (SEQ ID NO: 2) or a functional equivalent of this protein for use in a method of treating or preventing a proliferative retinal disease.

The invention also provides a method of treating or preventing a proliferative retinal disease in a subject, comprising administering a therapeutically or prophylactically effective amount of an agent which is a nucleic acid molecule encoding a protein comprising amino acids 19 to 168 of the amino acid sequence in FIG. 2 (SEQ ID NO: 2) or a functional equivalent of this protein.

The invention also provides an agent which is a nucleic acid molecule encoding a protein comprising amino acids 19 to 168 of the amino acid sequence in FIG. 2 (SEQ ID NO: 2) or a functional equivalent of this protein for use in a method of treating or preventing a proliferative retinal disease.

The invention also provides a method of treating or preventing a proliferative retinal disease in a subject, which comprises administering (a) a therapeutically or prophylactically effective amount of an agent which is a protein comprising amino acids 19 to 168 of the amino acid sequence in FIG. 2 (SEQ ID NO: 2) or a functional equivalent of this protein and (b) a second proliferative retinal disease treatment to said subject.

The invention also provides (a) an agent which is a protein comprising amino acids 19 to 168 of the amino acid sequence in FIG. 2 (SEQ ID NO: 2) or a functional equivalent of this protein and (b) a second proliferative retinal disease treatment, for use in a method of treating or preventing a proliferative retinal disease.

The invention also provides a method of treating or preventing a proliferative retinal disease in a subject comprising administering (a) a therapeutically or prophylactically effective amount of an agent which is a nucleic acid molecule encoding a protein comprising amino acids 19 to 168 of the amino acid sequence in FIG. 2 (SEQ ID NO: 2) or a functional equivalent of this protein and (b) a second proliferative retinal disease treatment to said subject.

The invention also provides (a) an agent which is a nucleic acid molecule encoding a protein comprising amino acids 19 to 168 of the amino acid sequence in FIG. 2 (SEQ ID NO: 2) or a functional equivalent of this protein and (b) a second proliferative retinal disease for use in a method of treating or preventing a proliferative retinal disease.

The invention also provides a method of reducing the amount of a second proliferative retinal disease treatment that is required to treat or a proliferative retinal disease, or reducing the duration of treatment with a second proliferative retinal disease treatment that is required to treat or prevent a proliferative retinal disease, or increasing the amount of time between consecutive treatments with said second proliferative retinal disease treatment, said method comprising administering a therapeutically or prophylactically effective amount of an agent which is a protein comprising amino acids 19 to 168 of the amino acid sequence in FIG. 2 (SEQ ID NO: 2) or a functional equivalent of this protein, or a nucleic acid molecule encoding said agent , and said second proliferative retinal disease treatment.

Example 4 shows that nomacopan-type proteins can effectively reduce VEGF levels in a mouse model of EAU. As nomacopan and its variants are not known to have any direct effect on VEGF, it is suggested that this is an indirect effect through the inhibition of LTB4 activation of the M2 macrophages referred to above, which are thought to be the principal source of VEGF in this model.

The agent and method of the invention therefore can prevent or treat proliferative retinal diseases, as described elsewhere herein, through the reduction of VEGF levels, e.g. in retinal tissue. For example, by reduction of VEGF signalling in retinal tissue. Effects of such a reduction, in turn, give rise to e.g. a reduction of angiogenesis and/or a reduction of vascular permeability.

The agent and method of the invention can also prevent or treat proliferative retinal diseases, as described elsewhere herein, through the reduction of LTB4-activation of M2 macrophages.

### DETAILED DESCRIPTION

Diseases

Proliferative retinal diseases are retinal conditions that involve the formation of blood vessels on the retina. For example, blood vessels can be produced in response to reduced blood supply caused by retinal ischaemia. This neovasculation occurs in general in response to the growth hormone Vascular Endothelial Cell Growth Factor (VEGF), which stimulates the production of new blood vessels on the optic disc or on the retinal surface. However, these new blood vessels are particularly weak, prone to leaking and can easily rupture resulting in haemorrhage and severe visual loss. Possible proliferative retinal diseases include autoimmune uveitis, infective uveitis, wet age-related macular degeneration (choroidal neovascularisation), dry age-related macular degeneration (geographic atrophy), diabetic retinopathy, diabetic macular oedema, optic neuritis (e.g. glaucoma associated), retinal vein occlusion and retinopathy of prematurity. Other proliferative retinal diseases include Stargardt disease and polypoidal choroidal vasculopathy.

The inventors have tested nomacopan, L-nomacopan, PAS-nomacopan and PAS-L-nomacopan in experiments in a mouse model of experimental autoimmune uveitis. Certain molecules were administered topically. Certain molecules were administered intravitreally. It was shown that nomacopan-type proteins can reduce the clinical score in this mouse model, as set out in Examples 1 and 3. The data presented below also demonstrated that these molecules can reduce the population of Th-17 cells and thereby reduce IL-17 levels. The inventors have also shown that LTB4 receptors (BLT1) can be observed on cells infiltrating into the mouse retina in this mouse model. Furthermore, these nomacopan-type molecules can bind to LTB4 and inhibit its action.

The ability of the tested molecules to reduce LTB4 signalling is consistent with the observation that upon retinal damage M2 macrophages migrate and attach to the damaged areas of the retina. These M2 macrophages subsequently release VEGF in response to LTB4 stimulation, which stimulates the production of new blood vessels [35]. So without wishing to be bound by any theory the administration of nomacopan-type proteins is believed to result in a reduction in the levels of VEGF, which reduces and/or prevents the production of new blood vessels. This theory is consistent with the data in Example 4 and FIG. 7A, which demonstrates that in a mouse model of EAU, the administration of nomacopan-type proteins significantly reduces the levels of VEGF in the retinal tissue.

Therefore, nomacopan-type proteins may be useful in the treatment or prevention of proliferative retinal diseases. Therefore, the invention provides a method for preventing or treating proliferative retinal diseases in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. In addition, the invention provides an agent which is a protein comprising amino acids 19 to 168 of the amino acid sequence in FIG. 2 (SEQ ID NO: 2) or a functional equivalent of this protein for use in preventing or treating proliferative retinal diseases.

The presence of these diseases may be determined by routine diagnosis that is well understood in the art. The severity of certain conditions can also be scored, which is useful in assessing whether a certain treatment is effective.

Uveitis

Autoimmune uveitis is an inflammatory process of the uveal components (the iris, ciliary body and choroid) due to an autoimmune reaction to self-antigens or caused by an innate inflammatory reaction secondary to an external stimulus. It can be present in different anatomical forms—anterior, intermediate, posterior or diffuse. Anterior uveitis is the most common form of the disease, which manifests as iritis that affects the iris, or iridociclitis which affects the ciliary body. Intermediate uveitis or vitritis involves the vitreous cavity and may involve the pars plan and posterior uveitis is divided in three types: choroiditis, retinochoroiditis, and chorioretinitis. Chorioretinitis is usually associated with infective diseases such as toxoplasmosis. In diffuse involvement or when uveitis affects many areas, it is described as panuveitis.

The type of uveitis can be classified used the International Uveitis Study Group (IUSG) Classification and the Standardization of Uveitis Nomenclature (SUN) group can be used to define the criteria for the onset, duration, and course of uveitis [27]. Uveitis predominantly affects people aged 20 to 50 years; although it can occur at any age and even affects children. Uveitis rates are also high in patients aged 65 or older.

Nomacopan-type proteins have been shown to reduce clinical scores and histological scores in a mouse model of autoimmune uveitis (EAU), as shown in Examples 1 and 3.

Furthermore Th17 cells, a CD4+ T-cell subset, produce interleukin (IL)-17, a pro-inflammatory cytokine that has been shown to be involved in several forms of infectious and noninfectious uveitis. IL-17 induces the production of other inflammatory cytokines such as IL-6, granulocyte colony-stimulating factor (CSF), granulocyte-macrophage-CSF, IL-1, TGF-β, and tumor necrosis factor (TNF)-α [28]. The examples also demonstrate that nomacopan-type proteins can decrease the percentage of CD4+ cells which express IL-17. Without wishing to be bound by any particular theory, these molecules may reduce the levels of IL-17 producing Th17 cells, which results in reduced inflammation in the uvea, thereby reducing the progression of uveitis. Nomacopan-type proteins may therefore be particularly useful in the treatment or prevention of autoimmune uveitis or infective uveitis.

VEGF plays an important role in the inflammatory process by promoting angiogenesis and increases vascular permeability. The expression of VEGF is linked to a number of major cytokines in the inflammtory cascade, including NFκB. The importance of VEGF in the development of retinal neovascularisation is well-established [29]. Nomacopan-type proteins can bind to LTB4 and inhibit its action, which is proposed to reduce the level of VEGF expression by M2 macrophages. This is demonstrated in the EAU mouse model in Example 4 and FIG. 7A, which shows that nomacopan-type proteins decreases VEGF levels in retinal tissue. The resulting decrease in the levels of VEGF prevent the production of new blood vessels. Nomacopan-type proteins may, therefore, be useful in the treatment or prevention of autoimmune uveitis or infective uveitis.

In preferred embodiments, the invention provides a method for preventing or treating autoimmune uveitis in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. In addition, the invention provides an agent which is a protein comprising amino acids 19 to 168 of the amino acid sequence in FIG. 2 (SEQ ID NO: 2) or a functional equivalent of this protein for use in preventing or treating autoimmune uveitis.

In certain embodiments, the invention provides a method for preventing or treating infective uveitis in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. In addition, the invention provides an agent which is a protein comprising amino acids 19 to 168 of the amino acid sequence in FIG. 2 (SEQ ID NO: 2) or a functional equivalent of this protein for use in preventing or treating infective uveitis.

In certain embodiments, the autoimmune uveitis can be anterior, intermediate, posterior or diffuse uveitis. In preferred embodiments, the invention provides a method for preventing or treating anterior uveitis in a subject. Also provided are compositions of the invention for use in preventing or treating anterior uveitis in a subject.

Subjects at risk of developing autoimmune uveitis may benefit from administration of the agents referred to herein, in order to prevent autoimmune uveitis. Risk factors for uveitis include smoking. Subjects who are smokers or who have been smokers are preferred, in terms of treatment or prevention of autoimmune uveitis. In some embodiments a subject may have one or more of these risk factors but may not show any clinical symptoms.

The subject to be treated can be aged between 20 to 50 years old. In some embodiments, the subject is older than 65 years old. In some embodiments, the subject is 18 years or older. In other embodiments, the subject under 18 years in age.

Age-Related Macular Degeneration

Age-related macular degeneration (AMD) is a degenerative retinal eye disease that causes a progressive, irreversible, severe loss of central vision. The disease impairs the macula, the region of highest visual acuity, and is one of the leading cause of blindness in Americans aged 60 years or older.

There are two types of AMD—wet and dry AMD. Wet AMD (also called neovascular AMD) is associated with rapidly deteriorating vision and severe impairment. Visual function is severely impaired in Wet AMD, and eventually inflammation and scarring cause permanent loss of visual function in the affected retina. Wet MD has two subtypes—‘classic’ and ‘occult’. In the classic subtype new blood vessels can be seen distinctly by an ophthalmologist using angiography, whereas in the occult subtype the leaking blood vessels are obscured. Patients may present with a combination of both occult and classic CNV [30].

Wet AMD is particularly characterized by abnormal neovascularization in and under the neuroretina in response to various stimuli. This abnormal vessel growth leads to the formation of leaky vessels which often haemorrhage. The abnormal blood vessel growth is activated by VEGF. Nomacopan-type proteins can bind to and inhibit LTB4 which is known to activate VEGF. Therefore, nomacopan-type proteins may be particularly effective at treating wet AMD.

Dry AMD (also called atrophic AMD) accounts for about 80% of cases and generally develops slowly, often affecting both eyes simultaneously. It usually causes only mild loss of vision. Dry AMD is characterised by fatty deposits behind the retina which cause the macula to thin and dry out.

AMD can be self-assessed using a STARS questionnaire [31]. AMD can be classified based on fundus lesions assessed within 2 disc diameters of the fovea in persons older than 55 years. Subjects with no visible drusen or pigmentary abnormalities should be considered to have no signs of AMD. Persons with small drusen (<63 μm), also termed drupelets, should be considered to have normal aging changes with no clinically relevant increased risk of late AMD developing. Persons with medium drusen (≥63-<125 μm), but without pigmentary abnormalities thought to be related to AMD, should be considered to have early AMD. Persons with large drusen or with pigmentary abnormalities associated with at least medium drusen should be considered to have intermediate AMD. Persons with lesions associated with neovascular AMD or geographic atrophy should be considered to have late AMD [32].

Increased local and systemic complement activation have been observed in AMD. Polymorphisms in a number of complement genes increase the risk of AMD [33]. Nomacopan is known to inhibit the complement activator C5. Therefore, nomacopan-type proteins that bind to C5 may be effective at treating AMD by inhibiting the activation of the complement pathway.

The LTB4 receptor has been shown to promote laser-induced choroidal neovascularization in a mouse model for wet-type AMD and the expression of VEGF mRNA has been spatially and temporally correlated with neovascularization in several animal models of retinal ischemia [34, 35]. As discussed previously, nomacopan-type proteins can inhibit LTB4. This may result in a reduction in the levels of VEGF, which prevents the production of new blood vessels. Nomacopan-type proteins may, therefore, be useful in the treatment or prevention of AMD.

Therefore, the invention provides a method for preventing or treating wet age-related macular degeneration in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. In other embodiments, the invention provides an agent which is a protein comprising amino acids 19 to 168 of the amino acid sequence in FIG. 2 (SEQ ID NO: 2) or a functional equivalent of this protein for use in preventing or treating wet age-related macular degeneration. The wet age-related macular degeneration can be occult, classic or a combination thereof.

Furthermore, the invention provides a method for preventing or treating dry age-related macular degeneration in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. In other embodiments, the invention provides an agent which is a protein comprising amino acids 19 to 168 of the amino acid sequence in FIG. 2 (SEQ ID NO: 2) or a functional equivalent of this protein for use in preventing or treating dry age-related macular degeneration.

In preferred embodiments, the subject to be treated is aged 60 years or old.

Subjects at risk of developing AMD may benefit from administration of the agents referred to herein, in order to prevent AMD. Risk factors for AMD include smoking, sunlight, artificial fats (such as partially-hydrogenated vegetable oils), a diet high in processed, packaged foods and low in fresh vegetables, uncontrolled hypertension and high cholesterol, diabetes, old age (patients over the age of 60 are at a greater risk than younger patients) and obesity.

Subjects having one or more of these risk factors are preferred, in terms of treatment or prevention of AMD. In some embodiments a subject may have one or more of these risk factors but may not show clinical symptoms.

Diabetic Retinopathy

Diabetes profoundly impacts the microvasculature in nearly every tissue. Diabetic retinopathy is characterized by microaneurysms, hard exudates, hemorrhages and venous abnormalities. Hyperglycemia induces microvascular retinal changes which leads to blurred vision, dark spots or flashing lights, and sudden loss of vision [36].

There are three different types of diabetic retinopathy—background retinopathy, diabetic maculopathy and proliferative retinopathy. Background retinopathy, also known as simple retinopathy, involves tiny swellings in the walls of the blood vessels. Known as blebs, they show up as small dots on the retina and are usually accompanied by yellow patches of exudates (blood proteins). Diabetic maculopathy is when the macula sustains some form of damage. One such cause of macular damage is from diabetic macular oedema whereby blood vessels near to the macula leak fluid or protein onto the macula. Proliferative retinopathy is an advanced stage of diabetic retinopathy in which the retina becomes blocked causing the growth of abnormal blood vessels. These can then bleed into the eyes, cause the retina to detach, and seriously damage vision. If left untreated, this can cause blindness [36].

VEGF is an important factor in the development of diabetic retinopathy. Nomacopan-type proteins can bind to and inhibit LTB4, which may result in a reduction in the levels of VEGF. Therefore, nomacopan-type proteins may be useful in the treatment or prevention of diabetic retinopathy.

Therefore, the invention provides a method for preventing or treating diabetic retinopathy in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. Furthermore, the invention provides an agent which is a protein comprising amino acids 19 to 168 of the amino acid sequence in FIG. 2 (SEQ ID NO: 2) or a functional equivalent of this protein for use in preventing or treating diabetic retinopathy. The diabetic retinopathy can be background retinopathy, diabetic maculopathy or proliferative retinopathy.

The subject in need of treatment can have type 1 or type 2 diabetes. The longer a subject suffers from diabetes and if the blood sugar levels are poorly controlled the higher the probability of suffering from diabetic retinopathy. In some embodiments, the subject has suffered from diabetes for at least 5, 10, 20, 30 or 40 years.

Other risk factors include high blood pressure, high cholesterol, pregnancy, tobacco use and being African-American, Hispanic or Native American.

Subjects at risk of developing diabetic retinopathy may benefit from administration of the agents referred to herein, in order to prevent diabetic retinopathy. Subjects having one or more of these risk factors are preferred, in terms of treatment or prevention of diabetic retinopathy. In some embodiments a subject may have one or more of these risk factors but may not show clinical symptoms.

Optic Neuropathy

Optic neuropathy occurs after damage to the optic nerve. The classic clinical signs of optic neuropathy are visual field defect, dyschromatopsia, and abnormal papillary response. The main symptom is loss of vision, with colours appearing subtly washed out in the affected eye. In many cases, only one eye is affected and patients may not be aware of the loss of colour vision until the doctor asks them to cover the healthy eye.

The rapid onset of optic neuropathy is characteristic of optic neuritis, ischemic optic neuropathy, inflammatory (non-demyelinating) and traumatic optic neuropathy. A gradual progression of symptoms is observed in compressive toxic/nutritional optic neuropathy.

There are several types of optic neuropathies including:
(a) Ischemic optic neuropathies, where there is insufficient blood flow to the optic nerve. These include anterior ischemic optic neuropathies that affect the optic nerve head and cause swelling of the optic disc and posterior ischemic optic neuropathies that do not involve the disc swelling;
(b) Optic neuritis, which is inflammation of the optic nerve and is associated with swelling and destruction of the myelin sheath covering the optic nerve. Optic neuritis can be classified into single isolated optic neuritis, relapsing isolated optic neuritis, chronic relapsing inflammatory optic neuropathy, neuromyelitis optica spectrum disorder, multiple sclerosis associated optic neuritis and classified optic neuritis forms. Optic neuritis can also be associated with glaucoma;
(c) Compressive optic neuropathy, which results from tumours, infections and inflammatory processes that cause lesions within the orbit and, less commonly, the optic canal. The lesions compress the optic nerve resulting optic disc swelling and progressive visual loss. Implicated orbital disorders include optic gliomas, meningiomas, hemangiomas, lymphangiomas, dermoid cysts, carcinoma, lymphoma, multiple myeloma, inflammatory orbital pseudotumor, and thyroid ophthalmopathy;
(d) Infiltrative optic neuropathy, where the optic nerve is be infiltrated by a variety of processes, including tumors, inflammation, and infections. The most common inflammatory disorder that infiltrates the optic nerve is sarcoidosis. Opportunistic fungi, viruses, and bacteria may also infiltrate the optic nerve. The optic nerve may be elevated if the infiltration occurs in the proximal portion of the nerve. The appearance of the nerve on examination depends on the portion of the nerve that is affected;
(e) Traumatic optic neuropathy, where the optic nerve is be damaged when exposed to direct or indirect injury. Falls are also a common cause, and optic neuropathy most commonly occurs when there is a loss of consciousness associated with multi-system trauma and serious brain injury;
(f) Mitochondrial optic neuropathies. Mitochondria play a central role in maintaining the life cycle of retinal ganglion cells because of their high energy dependence. Genetic mutations in mitochondrial DNA, vitamin depletion, alcohol and tobacco abuse, and use of certain drugs can cause derangements in efficient transport of mitochondria, which can cause a primary or secondary optic neuropathy;
(g) Nutritional optic neuropathies, which result from a lack of nutrition in the patient's diet. Nutritional deficiencies affect the whole body, so pain or loss of sensation in the arms and legs (peripheral neuropathy) is often seen in patients with nutritional optic neuropathies;
(h) Toxic optic neuropathies. The most common cause of which is methanol intoxication;
(i) Hereditary optic neuropathies, which typically manifest as symmetric bilateral central visual loss. Possible hereditary optic neuropathies include: Leber's hereditary optic neuropathy, dominant optic atrophy, Behr' s syndrome and Berk-Tabatznik syndrome.

Therefore, the invention provides a method for preventing or treating an optic neuropathy condition in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. Furthermore, the invention provides an agent which is a protein comprising amino acids 19 to 168 of the amino acid sequence in FIG. 2 (SEQ ID NO: 2) or a functional equivalent of this protein for use in preventing or treating an optic neuropathy condition.

In some embodiments, the optic neuropathy condition comprises any condition in which the optic nerve is damaged. The optic neuropathy condition may be selected from: optic neuritis, ischemic optic neuropathy, compressive optic neuropathy, infiltrative optic neuropathy, traumatic optic neuropathy, mitochondrial optic neuropathy, nutritional optic neuropathy, toxic optic neuropathy and hereditary optic neuropathy. In preferred embodiments the optic neuropathy condition is glaucoma associated optic neuritis.

Retinal Vein Occlusion

Retinal vein occlusion is a vascular disorder of the retina. It is the second most common cause of blindness after diabetic retinopathy and occurs mostly in patients over 60 years old.

There are three types of retinal vein occlusion. The first is branch retinal vein occlusion caused by a blockage in one of the four retinal veins. the second is central retinal vein occlusion which is caused by an obstruction of the main retinal vein and the third is branch retinal vein occlusion, where the obstruction occurs at a distal branch of the retinal vein. Central retinal vein occlusion usually results in more severe vision loss. Retinal vein occlusion can be further subdivided into nonischemic and ischemic types, depending on the amount of retinal capillary ischemia [37].

Retinal vein occlusion can be diagnosed using optical coherence tomography. This involves taking a high definition image of the retina using a scanning ophthalmoscope with a resolution of 5 microns. These images can determine the presence of swelling and edema by measuring the thickness of your retina. An ophthalmoscopy and fluorescein angiography can also be used to diagnose retinal vein occlusion by examining the retina and retinal blood vessels, respectively.

The two main complications of retinal vein occlusion are macular oedema and retinal ischaemia leading to iris and retinal neovascularisation. After a blockage occurs in the renal vein pressure builds up in the capillaries, leading to hemorrhage and leakage of fluid and blood. Neovascularization, new abnormal blood vessel growth, then occurs, which can result in neovascular glaucoma, vitreous hemorrhage, and, in late or severe cases, retinal detachment [37]. VEGF has a leading role in retinal vein occlusion pathogenesis as if ischaemia develops the VEGF is secreted, which results in further vascular leakage and retinal oedema [38].

Nomacopan-type proteins can bind to and inhibit LTB4, which may result in a reduction in the levels of VEGF. Therefore, nomacopan-type proteins may be useful in the treatment or prevention of diabetic retinopathy. Therefore, the invention provides a method for preventing or treating retinal vein occlusion in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. Furthermore, the invention provides an agent which is a protein comprising amino acids 19 to 168 of the amino acid sequence in FIG. 2 (SEQ ID NO: 2) or a functional equivalent of this protein for use in preventing or treating retinal vein occlusion. The retinal vein occlusion can be branch retinal vein occlusion, central retinal vein occlusion or hemicentral retinal vein occlusion.

In some embodiments, the subject is 60 years or older, 65 years or older, 70 years or older or 80 years or older. In preferred embodiments, the the subject is 65 years or older.

Retinopathy of Prematurity

Retinopathy of prematurity (ROP) is one of the leading causes of childhood blindness, which is characterized by retinal neovascularization that can eventually lead to tractional retinal detachment. ROP affects around 20 percent of babies who are born prematurely. It mainly occurs in babies who are born before week 32 of pregnancy or weigh less than 1500g when they are born.

ROP has no outward symptoms, therfore all premature babies born before week 32 of pregnancy or weighing less than 1.5 kg are screened by an ophthalmologist on a weekly or two-weekly basis. The extent and severity of ROP are traditionally described in terms of location (zones; I to III), severity (stages; 1 to 5), extent (clockhours; 1 to 12), and vascular dilatation and tortuosity (plus disease) according to the International Classification of ROP definition [39].

The blood vessels normally develop between 16-36 weeks of pregnancy and VEGF plays a key role in the angiogenesis of the foetus. During normal development VEGF is released in response to the higher oxygen demand of the retinal tissue, which leads to the development of blood vessels. However, in premature infants the levels of VEGF are elevated which leads to abnormal vascular proliferation [40].

Therefore, the invention provides a method for preventing or treating retinopathy of prematurity in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. Furthermore, the invention provides an agent which is a protein comprising amino acids 19 to 168 of the amino acid sequence in FIG. 2 (SEQ ID NO: 2) or a functional equivalent of this protein for use in preventing or treating retinopathy of prematurity.

In some embodiments, the subject is a premature baby born before 27 weeks gestational age, born between 27 and 32 weeks gestational age or born >32 weeks gestational age but with birthweight <1501 grams.

Pre-Proliferative Retinal Diseases

The subject may have, be suspected of having, or may be at risk of developing proliferative retinal diseases. For example, the subject may have a pre-proliferative retinopathy. Pre-proliferative retinopathies indicate the occurrence of chronic retinal ischaemia due to blocked capillaries. The clinical signs of a pre-proliferative retinopathy include multiple cotton wool spots, venous beading and/or looping, multiple deep round and blot haemorrhages and intra-retinal microvascular abnormalities.

As mentioned previously, proliferative retinal diseases are characterised by neovasculation, which is activated by VEGF. VEGF promotes vascular permeability and angiogenesis, which can lead to abnormal production of blood vessels in proliferative retinal diseases. Nomacopan-type proteins can bind to and inhibit LTB4, which can reduce the level of VEGF. This is shown e.g. in Example 4.

Therefore, the invention provides a method for preventing or treating a pre-proliferative retinal disease in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. Furthermore, the invention provides an agent which is a protein comprising amino acids 19 to 168 of the amino acid sequence in FIG. 2 (SEQ ID NO: 2) or a functional equivalent of this protein for use in preventing or treating a pre-proliferative retinal disease.

Stargardt Disease

Stargardt disease is an inherited macular dystrophy caused by mutations in the ABCA4 gene encoding a retinal transporter protein. It is the most prevalent form of macular degeneration in children with an estimated prevalence of approximately 10 to 12.5 per 100,000 individuals in the United States. Patients with Stargardt disease develop severe vision loss within their first or second decades of life, which progresses to irreversible decreased visual acuity in almost all cases [41].

Pathology can include choroidal neovascularization, in which case intravitreal anti-VEGF injections are performed [42]. The present inventors have shown that nomacopan-type proteins can reduce VEGF levels in proliferative retinal diseases.

Therefore, the invention provides a method for preventing or treating Stargardt disease in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. Furthermore, the invention provides an agent which is a protein comprising amino acids 19 to 168 of the amino acid sequence in FIG. 2 (SEQ ID NO: 2) or a functional equivalent of this protein for use in preventing or treating Stargardt disease.

Polypoidal Choroidal Vasculopathy

Polypoidal choroidal vasculopathy (PCV) is a disease of the choroidal vasculature. It is present in both men and woman of many ethnicities, characterized by serosanguineous detachments of the pigmented epithelium and exudative changes that can commonly lead to subretinal fibrosis. Evidence supports that symptomatic patients with PCV can have complete regression without severe vision loss with photodynamic therapy and anti-VEGF treatment [43]. The present inventors have shown that nomacopan-type proteins can reduce VEGF levels in proliferative retinal diseases.

Therefore, the invention provides a method for preventing or treating polypoidal choroidal vasculopathy disease in a subject, which comprises administering a therapeutically or prophylactically effective amount of an agent which is a protein comprising amino acids 19 to 168 of the amino acid sequence in FIG. 2 (SEQ ID NO: 2) or a functional equivalent of this protein. Furthermore, the invention provides an agent which is a protein comprising amino acids 19 to 168 of the amino acid sequence in FIG. 2 (SEQ ID NO: 2) or a functional equivalent of this protein for use in preventing or treating polypoidal choroidal vasculopathy disease. In some embodiments, the agent is administered in combination with photodynamic therapy.

Outcomes of Administration

The subject may, as a result of the treatment, have reduced incidence of symptoms, alleviation of symptoms, inhibition or delay of occurrence or re-occurence of symptoms, or a combination thereof. Preferably the treatment gives rise to a reduction in the typical disease condition symptoms. For example, visual acuity is used as an endpoint in a number of clinical studies for proliferative retinal disease treatment. Treatment according to the invention may therefore give rise to an improvement in visual acuity.

As a result of the treatment the subject may exhibit an improvement in their clinical score, e.g. using one of the methods referred to above in relation to one of the specific diseases.

Furthermore, as a result of treatment the subject may exhibit reduced vascularisation or reduced vascular proliferation (e.g. within the eye). Other outcomes may include an improvement in visual acuity, a reduction in vision loss, an increase in visual recovery, a reduction in the central retina thickness and/or improvements in diabetic retinopathy severity scores. Furthermore, the treatment may result in a reduction in vitreous hemorrhages, neovascularization of the iris or angle, neovascular glaucomaa and/or retinal detachment. The reduction observed after administration of the active agent can be measured relative to a healthy individual, an individual with a more severe form of the relevant proliferative retinal disease or observed in the patient before treatment with the active agent.

The treatment may result in the reduction of retinal inflammation, a reduction in the number of Th17 cells, a reduction in the number of CD4+ cells expressing RORgt/Tbet (e.g. in uveitis).

The treatment may also result in a reduction in the amount of, or duration of, or frequency of treatment with a second disease treatment that is required.

Thus in a further embodiment of the invention, there is provided a method of improving visual acuity, improving clinical score, reducing vascularisation or vascular proliferation (e.g. within the eye), reducing vision loss, increasing visual recovery, reducting central retina thickness and/or improving diabetic retinopathy severity scores, reducing vitreous hemorrhages, reducing neovascularization of the iris or angle, reducing neovascular glaucomae and/or retinal detachment, reducing inflammation, reducing the number of Th17 cells, and/or reducing the number of CD4+ cells expressing RORgt/Tbet (e.g. in uveitis) in a subject with a retinal proliferation disease, said method comprising administering a therapeutically or prophylactically effective amount of an agent which is a protein comprising amino acids 19 to 168 of the amino acid sequence in FIG. 2 (SEQ ID NO: 2) or a functional equivalent of this protein, or a nucleic acid molecule encoding said agent. This may be alone or with a second retinal proliferative disease treatment.

The invention also provides an agent which is a protein comprising amino acids 19 to 168 of the amino acid sequence in FIG. 2 (SEQ ID NO: 2) or a functional equivalent of this protein, a nucleic acid molecule encoding said agent, for use in a method of improving visual acuity, improving clinical score, reducing vascularisation or vascular proliferation (e.g. within the eye), reducing vision loss, increasing visual recovery, reducting central retina thickness and/or improving diabetic retinopathy severity scores, reducing vitreous hemorrhages, reducing neovascularization of the iris or angle, reducing neovascular glaucomae and/or retinal detachment, reducing inflammation, a reducing the number of Th17 cells, and/or reducing the number of CD4+ cells expressing RORgt/Tbet (e.g. in uveitis) in a subject with a retinal proliferation disease.

In a further embodiment of the invention, there is provided a method of treating or preventing a proliferative retinal disease in a subject, said method comprising administering a therapeutically or prophylactically effective amount of an agent which is a protein comprising amino acids 19 to 168 of the amino acid sequence in FIG. 2 (SEQ ID NO: 2) or a functional equivalent of this protein, or a nucleic acid molecule encoding said agent, wherein the agent gives rise to a reduction in VEGF levels, e.g. in retinal tissue, and/or the agent gives rise to a reduction of VEGF signalling, e.g. in retinal tissues.

The invention also provides an agent which is a protein comprising amino acids 19 to 168 of the amino acid sequence in FIG. 2 (SEQ ID NO: 2) or a functional equivalent of this protein, or a nucleic acid molecule encoding said agent, for use in a method of treating or preventing a proliferative retinal disease in a subject, wherein the agent gives rise to a reduction in VEGF levels, e.g. in retinal tissue, and/or the agent gives rise to a reduction of VEGF signalling, e.g. in retinal tissues.

The agent of the invention can be used in combination with other retinal proliferation disease treatments, as discussed above. The combination of the agent of the invention with the other (referred to here as a “second”) retinal proliferation disease treatment may be such that the amount of the second agent is reduced in comparison to the amount that is used in the absence of treatment with the agent of the invention, or the duration of the treatment with the second agent is reduced in comparison to the duration of treatment that is used in the absence of treatment with the agent of the invention or the frequency with with the second agent needs to be administered is reduced. This is advantagous in view of the side effects of certain known treatments. Therefore, there is also provided a method of reducing the amount of a second treatment, reducing the frequency of administration of a second treatment, or reducing the duration of the second treatment said method comprising administering a therapeutically or prophylactically effective amount of an agent which is a protein comprising amino acids 19 to 168 of the amino acid sequence in FIG. 2 (SEQ ID NO: 2) or a functional equivalent of this protein and optionally further comprising administering said second treatment.

Where a second retinal proliferation disease treatment is used, preferably it is selected from an anti-inflammatory medication, e.g. steroid such as corticosteroid, an IMT drug e.g. methotrexate, azathioprine, and mycophenolate, a BRM drug (e.g. an anti-TNFalpha agent, for example an antibody or fragment thereof that bind TNFalpha, such as infliximab or adalimumab), an anti-VEGF treatment such as an anti-TNFalpha antibody or fragment thereof (such as bevacizumab ranibizumab (Lucentis) an anti-VEGF aptamer (such as pegaptanib (Macugen)), or another VEGF antagonist such as aflibercept (Eylea, a recombinant fusion protein consisting of vascular endothelial growth factor (VEGF)-binding portions from the extracellular domains of human VEGF receptors 1 and 2, that are fused to the Fc portion of the human IgG1 immunoglobulin).

When the nomacopan-type protein and a second agent are used, they may be administered together or separately. The nomacopan-type protein may be administered first and the second retinal proliferation disease treatment may be administered second, or vice versa.

Thus, where the agent of the invention is used in combination with one or more other retinal proliferation disease treatments, e.g. in methods described as above, this can be described as (i) an agent which is a protein comprising amino acids 19 to 168 of the amino acid sequence in FIG. 2 (SEQ ID NO: 2) or a functional equivalent of this protein for use in a method of treating or preventing a retinal proliferation disease with a second retinal proliferation disease treatment, or (ii) as a second retinal proliferation disease treatment for use in a method of treating or preventing a retinal proliferation disease with an agent which is a protein comprising amino acids 19 to 168 of the amino acid sequence in FIG. 2 (SEQ ID NO: 2) or a functional equivalent of this protein, or (iii) an agent which is a protein comprising amino acids 19 to 168 of the amino acid sequence in FIG. 2 (SEQ ID NO: 2) or a functional equivalent of this protein and a second retinal proliferation disease treatment, for use in a method of treating or preventing a retinal proliferation disease. In each of (i) to (iii) said method comprises administering an agent which is a protein comprising amino acids 19 to 168 of the amino acid sequence in FIG. 2 (SEQ ID NO: 2) or a functional equivalent of this protein and administering a second retinal proliferation disease treatment.

In some embodiments the agent which is a protein comprising amino acids 19 to 168 of the amino acid sequence in FIG. 2 (SEQ ID NO: 2) or a functional equivalent of this protein is administered topically and the second retinal proliferation disease treatment is administered topically, or directly into the eye, e.g. intravitreally or suprachoroidally, preferably directly into the eye, e.g. intravitreally or suprachoroidally.

In some embodiments the agent which is a protein comprising amino acids 19 to 168 of the amino acid sequence in FIG. 2 (SEQ ID NO: 2) or a functional equivalent of this protein is administered directly into the eye, e.g. intravitreally or suprachoroidally, and the second retinal proliferation disease treatment is administered topically, or directly into the eye, e.g. intravitreally or suprachoroidally.

In some embodiments the second retinal proliferation disease treatment is also an agent which is a protein comprising amino acids 19 to 168 of the amino acid sequence in FIG. 2 (SEQ ID NO: 2) or a functional equivalent of this protein.

Where the treatment gives rise to a reduction in the amount or duration of the second retinal proliferative disease treatment, the reduction may be up to or at least 10, 20, 30, 40, 50, 60, 70, 80% compared to the amount of the second treatment that is used in the absence of the agent of the invention.

Where the treatment gives rise to a reduction in the in the frequency of the treatment with the second retinal proliferative disease treatment, this may result in an increase in the time between administration of the second retinal proliferative disease treatment of up to about 1, 2, 3, 4, 5, 6, 7 or 8 weeks.

Subjects

Preferred subjects, agents, doses and the like are as disclosed herein.

Any reference to any reduction or increase is a reduction or increase in a disease parameter is compared to said subject in the absence of the treatment. Preferably, the parameter can be quantitated and where this is the case the increase or decrease is preferably statistically significant. For example the increase or decrease may be at least 3, 5, 10, 15, 20, 30, 40, 50% or more compared to the parameter in the absence of treatment (e.g. before said treatment is started).

The subject to which the agent is administered in the practice of the invention is preferably a mammal, preferably a human. The subject to which the agent is administered is at risk of a retinal proliferation disease or has a retinal proliferation disease.

Methods of the invention may also comprise one or more additional steps of (i) determining whether the subject is at risk of or has a retinal proliferation disease, (ii) determining the severity of the retinal proliferation disease, which may be carried out before and/or after administration of the agent of the invention.

Agent to be Used in the Invention

According to one embodiment of the invention, the agent is nomacopan itself or a functional equivalent thereof. In the following, the term “a nomacopan-type protein” is used as shorthand for “a protein comprising amino acids 19 to 168 of the amino acid sequence shown in FIG. 2 (SEQ ID NO: 2) or a functional equivalent thereof”.

Nomacopan was isolated from the salivary glands of the tick Ornithodoros moubata. Nomacopan is an outlying member of the lipocalin family and is the first lipocalin family member shown to inhibit complement activation. Nomacopan inhibits the classical, alternative and lectin complement pathways by binding to C5 and preventing its cleavage by C5 convertase into C5a and C5b, thus inhibiting both the production of C5a, which is an active (e.g. proinflammatory) peptide, and the formation of the MAC. Nomacopan has been demonstrated to bind to C5 and prevent its cleavage by C5 convertase in rat, mouse and human serum with an IC50 of approximately 0.02 mg/ml.

A nomacopan-type protein may thus comprise or consist of amino acids 19 to 168 of the amino acid sequence in FIG. 2 (SEQ ID NO: 2) or amino acids 1 to 168 of the amino acid sequence in FIG. 2 (SEQ ID NO: 2). The first 18 amino acids of the protein sequence given in FIG. 2 form a signal sequence which is not required for C5 binding or for LTB4 binding activity and so this may optionally be dispensed with, for example, for efficiency of recombinant protein production.

C5 Binding Properties of Nomacopan

The nomacopan protein has been demonstrated to bind to C5 with a Kd of 1 nM, determined using surface plasmon resonance (SPR) [44]. Nomacopan-type peptides (e.g. functional equivalents of the nomacopan protein) preferably retain the ability to bind C5, conveniently with a Kd of less than 360 nM, more conveniently less than 300 nM, most conveniently less than 250 nM, preferably less than 200 nM, more preferably less than 150 nM, most preferably less than 100 nM, even more preferably less than 50, 40, 30, 20, or 10 nM, and advantageously less than 5 nM, wherein said Kd is determined using surface plasmon resonance, preferably in accordance with the method described in [44].

Nomacopan inhibits the classical complement pathway, the alternative complement pathway and the lectin complement pathway. Preferably, a nomacopan-type protein binds to C5 in such a way as to stabilize the global conformation of C5 but not directly block the C5 cleavage site targeted by the C5 convertases of the three activation pathways. Binding of nomacopan to C5 results in stabilization of the global conformation of C5 but does not block the convertase cleavage site. Functional equivalents of nomacopan also preferably share these properties.

C5 is cleaved by the C5 convertase enzyme (FIG. 1). The products of this cleavage include an anaphylatoxin C5a and a lytic complex C5b which promotes the formation of a complex of C5b, C6, C7, C8 and C9, also known as membrane attack complex (MAC). C5a is a highly pro-inflammatory peptide implicated in many pathological inflammatory processes including neutrophil and eosinophil chemotaxis, neutrophil activation, increased capillary permeability and inhibition of neutrophil apoptosis [45].

Monoclonal antibodies and small molecules that bind and inhibit C5 have been developed to treat various diseases [46], in particular PNH, psoriasis, rheumatoid arthritis, systemic lupus erythematosus and transplant rejection. However, some of these monoclonal antibodies do not bind to certain C5 proteins from subjects with C5 polymorphisms, and are thus ineffective in these subjects [47]. Preferably, the nomacopan-type protein binds to and inhibits cleavage of not only wild-type C5 but also C5 from subjects with C5 polymorphisms that render treatment by eculizumab ineffective, or reduce the efficacy of treatment with eculizumab). The term “C5 polymorphism” includes any version of C5 which has been changed by insertion, deletion, amino acid substitution, a frame-shift, truncation, any of which may be single or multiple, or a combination of one or more of these changes compared to the wild-type C5. In a human subject, wild-type C5 is considered the C5 protein with accession number NP_001726.2 ; version GI:38016947. Examples of C5 polymorphisms include polymorphisms at amino acid position 885, e.g. Arg885Cys (encoded by c.2653C>T) p.Arg885His (encoded by c.2654G>A) and Arg885Ser, which decrease the effectiveness of the mAb eculizumab [47].

The ability of an agent to bind C5, including C5 from subjects with C5 polymorphisms, e.g. C5 polymorphisms that render treatment by eculizumab ineffective, or reduce the efficacy of treatment with eculizumab may be determined by standard in vitro assays known in the art, for example by surface plasmon resonance or western blotting following incubation of the protein on the gel with labelled C5. Preferably, the nomacopan-type protein binds C5, either wild-type and/or C5 from subjects with C5 polymorphisms, e.g. C5 polymorphisms that render treatment by eculizumab ineffective, or reduce the efficacy of treatment with eculizumab, with a Kd of less than 360 nM, more conveniently less than 300 nM, most conveniently less than 250 nM, preferably less than 200 nM, more preferably less than 150 nM, most preferably less than 100 nM, even more preferably less than 50, 40, 30, 20, or 10 nM, and advantageously less than 5 nM, wherein said Kd is determined using surface plasmon resonance, preferably in accordance with the method described in [44].

It may show higher, lower or the same affinity for wild-type C5 and C5 from subjects with C5 polymorphisms, e.g. C5 polymorphisms that render treatment by eculizumab ineffective, or reduce the efficacy of treatment with eculizumab.

The ability of a nomacopan-type protein to inhibit complement activation may also be determined by measuring the ability of the agent to inhibit complement activation in serum. For example, complement activity in the serum can be measured by any means known in the art or described herein.

LTB4 Binding Properties of Nomacopan

The nomacopan-type protein may also be defined as having the function of inhibiting eicosanoid activity. Nomacopan has also been demonstrated to bind LTB4. Functional equivalents of the nomacopan protein may also retain the ability to bind LTB4 with a similar affinity as the nomacopan protein.

The ability of a nomacopan-type protein to bind LTB4 may be determined by standard in vitro assays known in the art, for example by means of a competitive ELISA between nomacopan and anti-LTB4 antibody competing for binding to labelled LTB4, by isothermal titration calorimetry or by fluorescence titration. Data obtained using fluorescence titration shows that nomacopan binds to LTB4 with a Kd of between 200 and 300 pM. For example, binding activity for LTB4 (Caymen Chemicals, Ann Arbor, Mich., USA) in phosphate buffered saline (PBS) can be quantified in a spectrofluorimeter e.g. a LS 50 B spectrofluorimeter (Perkin-Elmer, Norwalk, Conn., USA). This may be carried out as follows:

Purified 100 nM solutions of nomacopan, in 2 mL PBS were applied in a quartz cuvette (10 mm path length; Hellma, Mülheim, Germany) equipped with a magnetic stirrer. Temperature was adjusted to 20° C. and, after equilibrium was reached, protein Tyr/Trp fluorescence was excited at 280 nm (slit width: 15 nm). The fluorescence emission was measured at 340 nm (slit width: 16 nm) corresponding to the emission maximum. A ligand solution of 30 μM LTB4 in PBS was added step-wise, up to a maximal volume of 20 ≤L (1% of the whole sample volume), and after 30 s incubation steady state fluorescence was measured. For calculation of the KD value, data was normalized to an initial fluorescence intensity of 100%, the inner filter effect was corrected using a titration of 3 μM N-acetyl-tryptophanamide solution and data was plotted against the corresponding ligand concentration. Then, non-linear least squares regression based on the law of mass action for bimolecular complex formation was used to fit the data with Origin software version 8.5 (OriginLab, Northampton, Mass., USA) using a published formula (Breustedt et al., 2006) [48].

Nomacopan may bind LTB4 with an with a Kd of less than 1 nM, more conveniently less than 0.9 nM, most conveniently less than 0.8 nM, preferably less than 0.7 nM, more preferably less than 0.6 nM, most preferably less than 0.5 nM, even more preferably less than 0.4 nM, and advantageously less than 0.3 nM, wherein said Kd is determined using fluorescence titration, preferably in accordance with the method above. The nomacopan-type protein preferably shares these properties.

Molecules That Bind to Both C5 and LTB4

According to one embodiment of the invention, the nomacopan-type protein may bind to both C5 and to LTB4 (e.g. to both wild-type C5 and C5 from subjects with C5 polymorphisms that render treatment by eculizumab ineffective, or reduce the efficacy of treatment with eculizumab, and to LTB4).

The nomacopan-type protein may thus act to prevent the cleavage of complement C5 by C5 convertase into complement C5a and complement C5b, and also to inhibit LTB4 activity. Using an agent which binds to both C5 and LTB4 can be advantageous. C5 and the eicosanoid pathway may both contribute to the observed pathology in proliferative retinal diseases. Thus by using a single agent which inhibits multiple pathways involved in the proliferative retinal diseases an enhanced effect may be achieved, compared to using an agent which inhibits only a single pathway. There are furthermore practical advantages associated with administering a single molecule.

Molecules That Bind to LTB4 But Which do Not Bind or Which Show Reduced Binding to C5

Nomacopan-type proteins which do not bind or which show reduced binding to C5, but which do retain LTB-4-binding activity are disclosed, for instance, in WO2018/193121, the entire contents of which are incorporated herein by reference. Such nomacopan-type proteins which have reduced or absent C5-binding activity but which retain LTB-4-binding ability may be used in all aspects of the present invention.

Such nomacopan-type proteins which have reduced or absent C5-binding activity but which retain LTB-4-binding ability may comprise or consist of the following sequences:

SEQ ID NO: 22 (SEQ ID NO: 5 of WO2018/193121) is the amino acid sequence of a modified nomacopan in which SEQ ID NO: 4 has been modified to change Met114 to Gln, Met116 to Gln, Leu117 to Ser, Asp118 to Asn, Ala119 to Gly, Gly120 to Ser, Gly121 to Ala, Leu122 to Asp, Glu123 to Asp and Val124 to Lys. (nomacopan variant 1)

SEQ ID NO: 23 (SEQ ID NO: 6 of WO2018/193121) is the amino acid sequence of a modified nomacopan in which SEQ ID NO: 4 has been modified to change Ala44 to Asn, Met116 to Gln, Leu117 to Ser, Gly121 to Ala, Leu122 to Asp, Glu123 to Ala and Asp149 to Gly. (nomacopan variant 2)

SEQ ID NO: 24 (SEQ ID NO: 7 of WO2018/193121) is the amino acid sequence of a modified nomacopan in which SEQ ID NO: 4 has been modified to change Ala44 to Asn, Met116 to Gln, Leu122 to Asp and Asp149 to Gly. (nomacopan variant 3)

SEQ ID NO: 25 (SEQ ID NO: 8 of WO2018/193121) is the amino acid sequence of a modified nomacopan in which SEQ ID NO: 4 has been modified to change Ala44 to Asn. (nomacopan variant 4).

The modified nomacopan polypeptides that exhibit a reduced ability to bind to C5 compared to the unmodified nomacopan polypeptide may in some preferred embodiments exhibit no detectable binding to C5.

C5 binding may, for example, be reduced by at least 2, 5, 10, 15, 20, 50, 100 fold, or eliminated relative to the binding exhibited by the unmodified nomacopan polypeptide in SEQ ID NO: 4.

In some embodiments C5 binding is reduced by at least 50%, 60%, 70%, 80%, 90% or 95% relative to the unmodified nomacopan polypeptide in SEQ ID NO: 4.

Such polypeptides may e.g. bind C5 with a KD greater than 1 micromolar as determined by Surface Plasma Resonance according to the method described in [49], or as set out in

Example 2 of WO2018193121 and/or may inhibit sheep red blood cell lysis by less than 10% when present at a concentration of 0.02 mg/mL in whole pooled normal serum with the CH50 lytic assay performed according to or similarly to that performed in [50]. The ability of the such polypeptides to bind to C5 may also be determined by measuring the ability of the agent to inhibit complement activation in serum.

In certain preferred embodiments variant 2 is used.

These molecules are examples of molecules which are functional variants of nomacopan which share the molecule's ability to bind LTB4, but which do not bind C5 or which have reduced binding to C5.

A functional equivalent of nomacopan may thus be a homologue or fragment of nomacopan which (i) retains its ability to bind to C5 and to prevent the cleavage of C5 by C5 convertase into C5a and C5b and/or (ii) which retains its ability to bind LTB4. In certain embodiments the functional equivalent has property (i) and (ii). In other embodiments the functional equivalent has property (ii), but reduced or no binding to C5 (e.g. one of Nomacompan variants 1 to 4).

In some embodiments, the agent of the invention is derived from a haematophagous arthropod. The term “haematophagous arthropod” includes all arthropods that take a blood meal from a suitable host, such as insects, ticks, lice, fleas and mites. Preferably, the agent is derived from a tick, preferably from the tick Ornithodoros moubata.

Homologues include paralogues and orthologues of the nomacopan sequence that is explicitly identified in FIG. 2, including, for example, the nomacopan protein sequence from other tick species, including Rhipicephalus appendiculatus, R. sanguineus, R. bursa, A. americanum, A. cajennense, A. hebraeum, Boophilus microplus, B. annulatus, B. decoloratus, Dermacentor reticulatus, D. andersoni, D. marginatus, D. variabilis, Haemaphysalis inermis, Ha. leachii, Ha. punctata, Hyalomma anatolicum anatolicum, Hy. dromedarii, Hy. marginatum marginatum, Ixodes ricinus, I. persulcatus, I. scapularis, I. hexagonus, Argas persicus, A. reflexus, Ornithodoros erraticus, O. moubata moubata, O. m. porcinus, and O. savignyi.

The term “homologue” is also meant to include the equivalent nomacopan protein sequence from mosquito species, including those of the Culex, Anopheles and Aedes genera, particularly Culex quinquefasciatus, Aedes aegypti and Anopheles gambiae; flea species, such as Ctenocephalides fells (the cat flea); horseflies; sandflies; blackflies; tsetse flies; lice; mites; leeches; and flatworms. The native nomacopan protein is thought to exist in O. moubata in another three forms of around 18 kDa and the term “homologue” is meant to include these alternative forms of nomacopan.

Methods for the identification of homologues of the nomacopan sequence given in FIG. 2 will be clear to those of skill in the art. For example, homologues may be identified by homology searching of sequence databases, both public and private. Conveniently, publicly available databases may be used, although private or commercially-available databases will be equally useful, particularly if they contain data not represented in the public databases. Primary databases are the sites of primary nucleotide or amino acid sequence data deposit and may be publicly or commercially available. Examples of publicly-available primary databases include the GenBank database (http://www.ncbi.nlm.nih.gov/), the EMBL database (http://www.ebi.ac.uk/), the DDBJ database (http://www.ddbj.nig.ac.jp/), the SWISS-PROT protein database (http://expasy.hcuge.ch/), PIR (http://pir.georgetown.edu/), TrEMBL (http://www.ebi.ac.uk/), the TIGR databases (see http://www.tigr.org/tdb/index.html), the NRL-3D database (http://www.nbrfa.georgetown.edu), the Protein Data Base (http://www.rcsb.org/pdb), the NRDB database (ftp://ncbi.nlm.nih.gov/pub/nrdb/README), the OWL database (http://www.biochem.ucl.ac.uk/bsm/dbbrowser/OWL/) and the secondary databases PROSITE (http://expasy.hcuge.ch/sprot/prosite.html), PRINTS (http://iupab.leeds.ac.uk/bmb5dp/prints.html), Profiles (http://ulrec3.unil.ch/software/PFSCAN_form.html), Pfam (http://www.sanger.ac.uk/software/pfam), Identify (http://dna.stanford.edu/identify/) and Blocks (http://www.blocks.fhcrc.org) databases. Examples of commercially-available databases or private databases include PathoGenome (Genome Therapeutics Inc.) and PathoSeq (previously of Incyte Pharmaceuticals Inc.).

Typically, greater than 30% identity between two polypeptides (preferably, over a specified region such as the active site) is considered to be an indication of functional equivalence and thus an indication that two proteins are homologous. Preferably, proteins that are homologues have a degree of sequence identity with the nomacopan protein sequence identified in FIG. 2 (SEQ ID NO:2) of greater than 60%. More preferred homologues have degrees of identity of greater than 70%, 80%, 90%, 95%, 98% or 99%, respectively with the nomacopan protein sequence given in FIG. 2 (SEQ ID NO:2). Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1]. The % identity may be over the full length of the relevant reference sequence (e.g. amino acids 1-168 of SEQ ID NO:2 or amino acids 19-168 of SEQ ID NO:2). Nomacopan-type proteins thus can be described by reference to a certain % amino acid sequence identity to a reference sequence e.g. amino acids 19-168 of FIG. 2, SEQ ID NO:2 or amino acids 1-168 of FIG. 2, SEQ ID NO:2 e.g. as a protein comprising or consisting of a sequence having at least 60%,70%, 80%, 90%, 95%, 98% or 99% identity to amino acids 19-168 of FIG. 2, SEQ ID NO:2 or amino acids 1-168 of FIG. 2, SEQ ID NO:2), Where the nomacopan-type protein comprises said sequence, the nomacopan-type protein may be a fusion protein (with e.g. a second protein, e,g. a heterologous protein). Suitable second proteins are discussed below.

In the various aspects and embodiments of this disclosure, the modified nomacopan polypeptides (e.g. nomacopan-type proteins) may differ from the unmodified nomacopan polypeptides in SEQ ID NO: 2 and SEQ ID NO: 4 by from 1 to 50, 2-45, 3-40, 4-35, 5-30, 6-25, 7-20, 8-25, 9-20, 10-15 amino acids, up to 1, 2, 3, 4, 5, 7, 8, 9, 10, 20, 30, 40, 50 amino acids. These may be substitutions, insertions or deletions but are preferably substitutions. Where deletions are made these are preferably deletions of up to 1, 2, 3, 4, 5, 7 or 10 amino acids, (e.g. deletions from the N or C terminus). Mutants thus include proteins containing amino acid substitutions, e.g. conservative amino acid substitutions that do not affect the function or activity of the protein in an adverse manner. This term is also intended to include natural biological variants (e.g. allelic variants or geographical variations within the species from which the nomacopan proteins are derived). Mutants with improved ability to bind wild-type C5 and/or C5 from subjects with a C5 polymorphism that render treatment by eculizumab ineffective, or reduce the efficacy of treatment with eculizumab) and/or LTB4 may also be designed through the systematic or directed mutation of specific residues in the protein sequence.

These modifications may be made to the nomacopan polypeptide as set out in SEQ ID NO: 2 and SEQ ID NO: 4 and the molecule will remain useful and will be considered to be a functional variant provided that the resulting modified nomacopan polypeptide retains (i) LTB4 binding activity and/or also (ii) C5 binding comparable with the nomacopan polypeptide as set out in SEQ ID NO: 2 and SEQ ID NO: 4, which can be determined e.g. using the tests referred to elsewhere herein (e.g. the binding to one or both of these is at least 80, 85, 90, 95% of the binding compared to the unmodified nomacopan polypeptide). As discussed elsewhere herein, both nomacopan and L-nomacopan have been shown to be effective in the treatment of a mouse model of autoimmune uveitis. L-nomacopan binds LTB4 but does not bind C5. Nomacopan like proteins may be defined by reference to their ability to bind to C5 and/or their ability to bind to LTB4. Those that bind LTB4 are of particular use in the invention. Those that bind LTB4 and C5 are also of particular use in the invention.

Given the requirement for functional variants to bind LTB4 and optionally also C5, when modification are made, certain residues should be excluded from modification. These include conserved cysteine residues.

Other residues should be excluded from modification or, if substituted, should only be subject to conservative modification. These are the LTB4 binding residues. In embodiments where the functional variant binds LTB4 and C5 then the C5 binding residues as defined below should preferably also be excluded from modification or, if substituted, should preferaby only be subject to conservative modification. Given that the binding of LTB4 and C5 is relatively well understood it is possible to design a molecule that may have a percentage identity of around 65% to nomacopan but in which the changes are confined to residues which are not involved in LTB4 binding and optionally also C5 binding.

For LTB4 binding variants, in some embodiments each of the six cysteine amino acids at positions 6, 38, 100, 128, 129, 150 of the mature nomacopan molecule (e.g. as set out in SEQ ID NO: 4 which corresponds to residues 19 to 168 of the full length protein including the signal sequence) is retained and at least five, ten or fifteen or each of the LTB4 binding residues set out below is retained or is subject to a conservative modification.

For LTB4 binding variants, in some embodiments each of the six cysteine amino acids at positions 6, 38, 100, 128, 129, 150 of SEQ ID NO: 4 is retained and at least five, ten or fifteen or each of the LTB4 binding residues are retained or are subject to a conservative modification, wherein up to 2, 3, 4, 5, 10, 15, 20 of the LTB4 binding residues set out below are subject to a conservative modification.

For LTB4 binding variants, in some embodiments each of the six cysteine amino acids at positions 6, 38, 100, 128, 129, 150 of SEQ ID NO: 4 is retained and at least five, ten or fifteen or each of the LTB4 binding residues set out below is retained.

For LTB4 binding variants, in some embodiments each of the six cysteine amino acids at positions 6, 38, 100, 128, 129, 150 of SEQ ID NO: 4 is retained and each of the LTB4 binding residues set out below is retained or is subject to a conservative modification.

For LTB4 binding variants, in some embodiments each of the six cysteine amino acids at positions 6, 38, 100, 128, 129, 150 of SEQ ID NO: 4 is retained and each of the LTB4 binding residues set out below is retained or is subject to a conservative modification, wherein up to 2, 3, 4, 5, 10, 15, 20 of the LTB4 binding residues are subject to a conservative modification.

For LTB4 binding variants, in some embodiments each of the six cysteine amino acids at positions 6, 38, 100, 128, 129, 150 of SEQ ID NO: 4 is retained and each of the LTB4 binding residues set out below is retained.

For variants that bind C5 and LTB4, in some embodiments each of the six cysteine amino acids at positions 6, 38, 100, 128, 129, 150 of the mature nomacopan molecule (e.g. as set out in SEQ ID NO: 4 which corresponds to residues 19 to 168 of the full length protein including the signal sequence) is retained and at least five, ten or fifteen or each of the LTB4 binding residues are retained or are subject to a conservative modification and at least five, ten or fifteen or twenty or each of C5 binding residues set out below is retained or is subject to a conservative modification.

For variants that bind C5 and LTB4, each of the six cysteine amino acids at positions 6, 38, 100, 128, 129, 150 of SEQ ID NO: 4 is retained and at least five, ten or fifteen or each of the LTB4 binding residues and at least five, ten or fifteen or twenty or each of C5 binding residues set out below is retained or is subject to a conservative modification, wherein up to 2, 3, 4, 5, 10, 15, 20 of the LTB4 and C5 binding residues are subject to a conservative modification.

For variants that bind C5 and LTB4, in some embodiments each of the six cysteine amino acids at positions 6, 38, 100, 128, 129, 150 of SEQ ID NO: 4 is retained and at least five, ten or fifteen or each of the LTB4 binding residues and at least five, ten or fifteen or twenty or each of C5 binding residues set out below is retained.

For variants that bind C5 and LTB4, in some embodiments each of the six cysteine amino acids at positions 6, 38, 100, 128, 129, 150 of SEQ ID NO: 4 is retained and each of the LTB4 binding residues and each of C5 binding residues set out below is retained or is subject to a conservative modification.

For variants that bind C5 and LTB4, in some embodiments each of the six cysteine amino acids at positions 6, 38, 100, 128, 129, 150 of SEQ ID NO: 4 is retained and each of the LTB4 binding residues and each of C5 binding residues set out below is retained or is subject to a conservative modification, wherein up to 2, 3, 4, 5, 10, 15, 20 of the C5 and/or LTB4 binding residues are subject to a conservative modification.

For variants that bind C5 and LTB4, in some embodiments each of the six cysteine amino acids at positions 6, 38, 100, 128, 129, 150 of SEQ ID NO: 4 is retained and each of the LTB4 binding residues and each of C5 binding residues set out below is retained.

Modifications made outside of these regions may be conservative or non-conservative.

In each of these embodiments the spacing between these six cysteine amino acid residues is preferably retained to preserve the overall structure of the molecule (e.g. the molecule comprises six cysteine residues that are spaced relative to each other at a distance of 32 amino acids apart, 62 amino acids apart, 28 amino acids apart, 1 amino acid apart and 21 amino acids apart as arranged from the amino terminus to the carboxyl terminus of the sequence according to amino acids 1 to 168 of the amino acid sequence in FIG. 2).

LTB4 Binding Residues

Resides that are thought to be involved in binding to LTB4 and are preferably retained in unmodified form or are subject to conservative changes only in the sequence of any molecule that is modified relative to SEQ ID NO:2 or SEQ ID NO:4 are Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115 (numbering according to SEQ ID NO:4).

C5 Binding Residues

Resides that are thought to be involved in binding to C5 may be retained in unmodified form in the sequence of any molecule that is modified relative to SEQ ID NO:2 or SEQ ID NO:4 are Val26, Val28, Arg29, Ala44, Gly45, Gly61, Thr62, Ser97, His99, His101, Met 114, Met 116, Leu117, Asp118, Ala119, Gly120, Gly121, Leu122, Glu123, Val124, Glu125, Glu127, His146, Leu147 and Asp 149 (numbering according to SEQ ID NO:4). These residues are among those that are modified in the nomacopan variants that bind to LTB4 but which have been modified to reduce binding to C5.

LTB4 and/or C5 Binding Residues

There are two histidine residues involved in both LTB4 and C5 binding, His99 and His101. The list of residues involved in LTB4 and/or C5 binding is therefore Phe18, Tyr25, Val26, Val28, Arg29, Arg36, Leu39, Gly41, Pro43, Ala44, Gly45, Leu52, Val54, Met56, Phe58, Gly61, Thr62, Thr67, Trp69, Phe71, Gln87, Arg89, Ser97, His99, His101, Asp103, Met 114, Trp115, Met 116, Leu117, Asp118, Ala119, Gly120, Gly121, Leu122, Glu123, Val124, Glu125, Glu127, His146, Leu147 and Asp 149 (numbering according to SEQ ID NO:4).

Further Examples of Molecules That Bind LTB4 But Which Show Reduced or No Binding to C5

As discussed above, nomacopan-type proteins which do not bind or which show reduced binding to C5, but which do retain LTB-4-binding activity are disclosed, for instance, in WO2018/193121, the entire contents of which are incorporated herein by reference. Such nomacopan-type proteins which have reduced or absent C5-binding activity but which retain LTB-4-binding ability may be used in all aspects of the present invention.

Four exemplary nomacopan-type proteins which have reduced or absent C5-binding activity but which retain LTB-4-binding ability are disclosed in WO2018/193121, specifically proteins having the amino acid sequences as set out in SEQ ID NO: 22 (SEQ ID NO: 5 of WO2018/193121, variant 1), SEQ ID NO: 23 (SEQ ID NO: 6 of WO2018/193121, variant 2), SEQ ID NO: 24 (SEQ ID NO: 7 of WO2018/193121, variant 3) and SEQ ID NO: 25 (SEQ ID NO: 8 of WO2018/193121, variant 4). L-nomacopan as referred to in the present examples is variant 2.

Such proteins are nomacopan-type proteins, and are thus considered to be functional equivalents of nomacopan, however they share only the LTB4 binding properties thereof and have reduced or no binding to C5.

Such nomacopan-type proteins as defined in WO2018/193121 are described in more detail below and may be used in the present invention.

These proteins which have reduced or absent C5-binding activity but which retain LTB-4-binding ability may comprise or consist of the following sequences:

SEQ ID NO: 22 (SEQ ID NO: 5 of WO2018/193121) is the amino acid sequence of a modified nomacopan in which SEQ ID NO: 4 has been modified to change Met114 to Gln, Met116 to Gln, Leu117 to Ser, Asp118 to Asn, Ala119 to Gly, Gly120 to Ser, Gly121 to Ala, Leu122 to Asp, Glu123 to Asp and Val124 to Lys. (nomacopan variant 1)

SEQ ID NO: 23 (SEQ ID NO: 6 of WO2018/193121) is the amino acid sequence of a modified Coversin in which SEQ ID NO: 4 has been modified to change Ala44 to Asn, Met116 to Gln, Leu117 to Ser, Gly121 to Ala, Leu122 to Asp, Glu123 to Ala and Asp149 to Gly. (nomacopan variant 2)

SEQ ID NO: 24 (SEQ ID NO: 7 of WO2018/193121) is the amino acid sequence of a modified Coversin in which SEQ ID NO: 4 has been modified to change Ala44 to Asn, Met116 to Gln, Leu122 to Asp and Asp149 to Gly. (nomacopan variant 3)

SEQ ID NO: 25 (SEQ ID NO: 8 of WO2018/193121) is the amino acid sequence of a modified Coversin in which SEQ ID NO: 4 has been modified to change Ala44 to Asn. (nomacopan variant 4)

SEQ ID NO: 26 (SEQ ID NO: 9 of WO2018/193121) is the amino acid sequence of the loop between beta H and alpha2 at amino acid positions 114 to 124 of SEQ ID NO: 4 (amino acid positions 132-142 of SEQ ID NO: 2).

SEQ ID NO: 27 (SEQ ID NO: 10 of WO2018/193121) is the amino acid sequence of the loop between beta H and alpha2 at amino acid positions 114 to 124 of SEQ ID NO: 4 in nomacopan variant 1 (SEQ ID NO: 22).

SEQ ID NO: 28 (SEQ ID NO: 11 of WO2018/193121) is the amino acid sequence of the loop between beta H and alpha2 at amino acid positions 114 to 124 of SEQ ID NO: 4 in nomacopan variant 2 (SEQ ID NO: 23).

SEQ ID NO: 29 (SEQ ID NO: 12 of WO2018/193121) is the amino acid sequence of the loop between beta H and alpha2 at amino acid positions 114 to 124 of SEQ ID NO: 4 in nomacopan variant 3 (SEQ ID NO: 24).

The nomacopan-type polypeptides which have reduced or absent C5-binding activity but which retain LTB-4-binding ability may be described as modified nomacopan polypeptides (e.g which exhibit leukotriene or hydroxyeicosanoid binding activity and reduced or absent C5 binding). References to a “modified nomacopan polypeptide” are to be understood as a reference to a modified version of either SEQ ID NO: 2 or SEQ ID NO: 4 i.e. the nomacopan polypeptide with or without the 18 amino acid signal sequence seen at the N-terminus of SEQ ID NO: 2. They may therefore be considered to be nomacopan-type proteins.

Such polypeptides may exhibit leukotriene or hydroxyeicosanoid binding activity and reduced or absent C5 binding and can comprise SEQ ID NO: 4 in which from 1 to 30 amino acid substitutions are made, wherein

(i) in the positions 114 to 124 of SEQ ID NO: 4 one or more of the following substitutions (a)-(j) is made:

a. Met114 is replaced with Gln, Asp, Asn, Glu, Arg, Lys, Gly, Ala, Pro, His, or Thr;

b. Met116 is replaced with Gln, Asp, Asn, Glu, Arg, Lys, Gly, Ala, Pro, His, or Thr;

c. Leu117 is replaced with Ser, Asp, Asn, Glu, Arg, Lys, Gly, Ala, or Pro;

d. Asp118 is replaced with Asn, Gln, Arg, Lys, Gly, Ala, Leu, Ser, Ile, Phe, Tyr, Met Pro, His, or Thr;

e. Ala119 is replaced with Gly, Asp, Asn, Glu, Arg, Lys, Leu, Ile, Phe, Tyr, Met, Pro, or His;

f. Gly120 is replaced with Ser, Asp, Asn, Glu, Arg, Lys, Leu, Ile, Phe, Tyr, Met, Pro, or His;

g. Gly121 is replaced with Ala, Asp, Asn, Glu, Arg, Lys, Leu, Ile, Phe, Tyr, Met, Pro, or His;

h. Leu122 is replaced with Asp, Glu, Asn, Ala, Gln, Arg, Lys, Pro, or His;

i. Glu123 is replaced with Asp, Ala, Gln, Asn, Arg, Lys, Gly, Leu, Ser, Ile, Phe, Tyr, Pro, His, or Thr;

j. Val124 is replaced with Lys, Gln, Asn, Arg, Lys, Gly, Ala, Pro, His, or Thr; or/and

wherein

(ii) Ala44 in SEQ ID NO: 4 is replaced with Asn, Asp, Gln, Glu, Arg, Lys, Leu, Ile, Phe, Tyr, Met, Pro, or His;

or a fragment thereof in which up to five amino acids are deleted from the N terminus of the modified nomacopan polypeptide.

LK/E binding activity as used herein refers to the ability to bind to leukotrienes and hydroxyeicosanoids including but not limited to LTB4, B4 isoleukotrienes and any hydroxylated derivative thereof, HETEs, HPETEs and EETs. LTB4 binding is of particular interest.

The modified nomacopan polypeptides which have reduced or absent C5-binding activity but which retain LTB-4-binding ability may consist of SEQ ID NO: 2 or 4, modified in accordance with the description below, or may comprise SEQ ID NO: 2 or 4, modified in accordance with the description below.

The nomacopan polypeptide in SEQ ID NO: 2 and SEQ ID NO: 4 features a loop between beta H and alpha2 at amino acid positions 114 to 124 of SEQ ID NO: 4 (amino acid positions 132-142 of SEQ ID NO: 2). This loop has the sequence shown below:

-Met-Trp-Met-Leu-Asp-Ala-Gly-Gly-Leu-Glu-Val- (SEQ ID NO: 26)

The first Met is at position 114 of SEQ ID NO: 4 and at position 132 of SEQ ID NO: 2. In the modified nomacopan polypeptide which has reduced or absent C5-binding activity but which retains LTB-4-binding ability, the nomacopan polypeptide of SEQ ID NO: 2 or SEQ ID NO: 4 is modified such that at positions 114 to 124 of SEQ ID NO: 4 one or more of the following substitutions (a)-(j) is made:

a. Met114 is replaced with Gln, Asp, Asn, Glu, Arg, Lys, Gly, Ala, Pro, His, or Thr, preferably Gln or Ala;

b. Met116 is replaced with Gln, Asp, Asn, Glu, Arg, Lys, Gly, Ala, Pro, His, or Thr, preferably Gln or Ala;

c. Leu117 is replaced with Ser, Asp, Asn, Glu, Arg, Lys, Gly, Ala, or Pro, preferably Ser or Ala;

d. Asp118 is replaced with Asn, Gln, Arg, Lys, Gly, Ala, Leu, Ser, Ile, Phe, Tyr, Met Pro, His, or Thr, preferably Asn;

e. Ala119 is replaced with Gly, Asp, Asn, Glu, Arg, Lys, Leu, Ile, Phe, Tyr, Met, Pro, or His, preferably Gly or Asn;

f. Gly120 is replaced with Ser, Asp, Asn, Glu, Arg, Lys, Leu, Ile, Phe, Tyr, Met, Pro, or His, preferably Ser or Asn;

g. Gly121 is replaced with Ala, Asp, Asn, Glu, Arg, Lys, Leu, Ile, Phe, Tyr, Met, Pro, or His, preferably Ala or Asn;

h. Leu122 is replaced with Asp, Glu, Asn, Ala, Gln, Arg, Lys, Pro, or His, preferably Asp or Ala;

i. Glu123 is replaced with Asp, Ala, Gln, Asn, Arg, Lys, Gly, Leu, Ser, Ile, Phe, Tyr, Pro, His, or Thr, preferably Asp, Ala, Gln or Asn;

j. Val124 is replaced with Lys, Gln, Asn, Arg, Lys, Gly, Ala, Pro, His, or Thr, preferably Lys or Ala.

In the modified nomacopan polypeptide which has reduced or absent C5-binding activity but which retains LTB-4-binding ability the nomacopan polypeptide in SEQ ID NO: 2 or SEQ ID NO: 4 can be modified such that at positions 114 to 124 of SEQ ID NO: 4 one or more of the following substitutions (a)-(j) is made:

a. Met114 is replaced with Gln;

b. Met116 is replaced with Gln;

c. Leu117 is replaced with Ser;

d. Asp118 is replaced with Asn;

e. Ala119 is replaced with Gly;

f. Gly120 is replaced with Ser;

g. Gly121 is replaced with Ala;

h. Leu122 is replaced with Asp;

i. Glu123 is replaced with Asp, or Ala;

j. Val124 is replaced with Lys.

In the modified nomacopan polypeptide two, three, four, five, six, seven, eight, nine, or ten of the substitutions (a)-(j) are present. Preferably two or more, five or more, or eight or more of the substitutions (a)-(j) are present.

In the modified nomacopan polypeptide which has reduced or absent C5-binding activity but which retains LTB-4-binding ability the nomacopan polypeptide in SEQ ID NO: 2 or SEQ ID NO: 4 can be modified such that at positions 114 to 124 of SEQ ID NO: 4 the following substitutions are present:

a. Met114 is replaced with Gln;

b. Met116 is replaced with Gln;

c. Leu117 is replaced with Ser;

d. Asp118 is replaced with Asn;

e. Ala119 is replaced with Gly;

f. Gly120 is replaced with Ser;

g. Gly121 is replaced with Ala;

h. Leu122 is replaced with Asp;

i. Glu123 is replaced with Asp;

j. Val124 is replaced with Lys.

Optionally in the modified nomacopan polypeptide referred to above Trp115 is not substituted. A preferred modified nomacopan polypeptide has a loop between beta H and alpha2 at amino acid positions 114 to 124 of SEQ ID NO: 4 that has the sequence Gln-Trp-Gln-Ser-Asn-Gly-Ser-Ala-Asp-Asp-Lys (SEQ ID NO: 27).

In the modified nomacopan polypeptide which has reduced or absent C5-binding activity but which retains LTB-4-binding ability, the nomacopan polypeptide can be modified such that at positions 114 to 124 of SEQ ID NO: 4 the following substitutions are present:

a. Met116 is replaced with Gln, Asp, Asn, Glu, Arg, Lys, Gly, Ala, Pro, His, or Thr, preferably Gln;

b. Leu117 is replaced with Ser, Asp, Asn, Glu, Arg, Lys, Gly, Ala, or Pro, preferably Ser;

c. Gly121 is replaced with Ala, Asp, Glu, Arg, Lys, Leu, Ile, Phe, Tyr, Met, Pro, or His, preferably Ala;

d. Leu122 is replaced with Asp, Glu, Asn, Gln, Arg, Lys, Pro, or His, preferably Asp;

e. Glu123 is replaced with Asp, Ala, Gln, Asn, Arg, Lys, Gly, Leu, Ser, Ile, Phe, Tyr, Pro, His, or Thr, preferably Asp.

In more particular embodiments;

a. Met116 is replaced with Gln;

b. Leu117 is replaced with Ser;

c. Gly121 is replaced with Ala;

d. Leu122 is replaced with Asp;

e. Glu123 is replaced with Ala.

Optionally in this modified nomacopan polypeptide referred to above Trp 115 is not substituted. Optionally in this embodiment Met114, Trp 115, Asp118, Ala119, Gly120 and Val124 are not substituted, or are substituted with conservative substitutions as referred to elsewhere herein. A preferred modified nomacopan polypeptide which has reduced or absent C5-binding activity but which retains LTB-4-binding ability has a loop between beta H and alpha2 at amino acid positions 114 to 124 of SEQ ID NO: 4 that has the sequence Met-Trp-Gln-Ser-Asp-Ala-Gly-Ala-Asp-Ala-Val (SEQ ID NO: 28).

In the modified nomacopan polypeptide which has reduced or absent C5-binding activity but which retains LTB-4-binding ability, the nomacopan polypeptide can be modified such that at positions 114 to 124 of SEQ ID NO: 4 the following substitutions are present:

a. Met116 is replaced with Gln, Asp, Asn, Glu, Arg, Lys, Gly, Ala, Pro, His, or Thr, preferably Gln;

b. Leu122 is replaced with Asp, Glu, Asn, Gln, Arg, Lys, Pro, or His, preferably Asp;

In more particular embodiments;

a. Met116 is replaced with Gln;

b. Leu122 is replaced with Asp.

Optionally in this modified nomacopan polypeptide referred to above Trp 115 is not substituted. Optionally in this embodiment Met114, Trp 115, Leu117, Asp118, Ala119, Gly120, Gly121, Glu123 and Val124 are not substituted. A preferred modified nomacopan polypeptide which has reduced or absent C5-binding activity but which retains LTB-4-binding ability has a loop between beta H and alpha2 at amino acid positions 114 to 124 of SEQ ID NO: 4 that has the sequence Met-Trp-Gln-Leu-Asp-Ala-Gly-Gly-Asp-Glu-Val (SEQ ID NO: 29).

In the modified nomacopan polypeptide which has reduced or absent C5-binding activity but which retains LTB-4-binding ability the nomacopan polypeptide can be modified such that Ala44 in SEQ ID NO: 4 (Ala62 in SEQ ID NO: 2) is replaced with Asn, Asp, Gln, Glu, Arg, Lys, Leu, Ile, Phe, Tyr, Met, Pro, or His.

In preferred embodiments Ala44 in SEQ ID NO: 4 is replaced with Asn.

This substitution at position 44 of SEQ ID NO: 4 (or position 62 of SEQ ID NO: 2) may be made in combination with any of the other substitutions referred to herein.

In another modified nomacopan polypeptide which has reduced or absent C5-binding activity but which retains LTB-4-binding ability the nomacopan polypeptide can be modified such that at positions 114 to 124 of SEQ ID NO: 4 one or more of the following substitutions (a)-(j) is present:

a. Met114 is replaced with Gln, Asp, Asn, Glu, Arg, Lys, Gly, Ala, Pro, His, or Thr, preferably Gln or Ala, e.g. Gln;

b. Met116 is replaced with Gln, Asp, Asn, Glu, Arg, Lys, Gly, Ala, Pro, His, or Thr, preferably Gln or Ala e.g. Gln;

c. Leu117 is replaced with Ser, Asp, Asn, Glu, Arg, Lys, Gly, Ala, or Pro, preferably Ser or Ala, e.g. Ser;

d. Asp118 is replaced with Asn, Gln, Arg, Lys, Gly, Ala, Leu, Ser, Ile, Phe, Tyr, Met Pro, His, or Thr, preferably Asn;

e. Ala119 is replaced with Gly, Asp, Glu, Arg, Lys, Leu, Ile, Phe, Tyr, Met, Pro, or His, preferably Gly or Asn, e.g. Gly;

f. Gly120 is replaced with Ser, Asp, Glu, Arg, Lys, Leu, Ile, Phe, Tyr, Met, Pro, or His, preferably Ser or Asn, e.g. Ser;

g. Gly121 is replaced with Ala, Asp, Glu, Arg, Lys, Leu, Ile, Phe, Tyr, Met, Pro, or His preferably Ala or Asn, e.g. Ala;

h. Leu122 is replaced with Asp, Glu, Asn, Gln, Arg, Lys, Pro, or His, preferably Asp or Ala, e.g. Asp;

i. Glu123 is replaced with Asp, Ala, Gln, Asn, Arg, Lys, Gly, Leu, Ser, Ile, Phe, Tyr, Pro, His, or Thr, preferably Asp, Ala, Gln or Asn, e.g. Asp or Ala;

j. Val124 is replaced with Lys, Gln, Asn, Arg, Lys, Gly, Ala, Pro, His, or Thr, preferably Lys or Ala, e.g. Lys;

and additionally Ala44 in SEQ ID NO: 4 (Ala62 in SEQ ID NO: 2) is replaced with Asn, Asp, Gln, Glu, Arg, Lys, Leu, Ile, Phe, Tyr, Met, Pro, or His, preferably Asn.

In some modified nomacopan polypeptides which have reduced or absent C5-binding activity but which retain LTB-4-binding ability, the nomacopan polypeptide can be modified such that at positions 114 to 124 of SEQ ID NO: 4 the following substitutions are present:

a. Met116 is replaced with Gln;

b. Leu117 is replaced with Ser;

c. Gly121 is replaced with Ala;

d. Leu122 is replaced with Asp;

e. Glu123 is replaced with Ala;

and Ala44 in SEQ ID NO: 4 is replaced with Asn.

In preferred aspects of this embodiment the amino acid residues corresponding to positions 114 to 124 of SEQ ID NO: 4 are as set out in SEQ ID NO: 28.

In some modified nomacopan polypeptides which have reduced or absent C5-binding activity but which retain LTB-4-binding ability, the nomacopan polypeptide is modified such that at positions 114 to 124 of SEQ ID NO: 4 the following substitutions are present:

a. Met116 is replaced with Gln;

b. Leu122 is replaced with Asp;

and Ala44 in SEQ ID NO: 4 is replaced with Asn

In preferred aspects of this embodiment the amino acid residues corresponding to positions 114 to 124 of SEQ ID NO: 4 are as set out in SEQ ID NO: 29.

In some modified nomacopan polypeptides which have reduced or absent C5-binding activity but which retain LTB-4-binding ability the Coversin polypeptide can be modified such that Asp149 in SEQ ID NO: 4 is replaced with Gly, Gln, Asn, Ala, Met, Arg, Lys, Leu, Ser, Ile, Phe, Tyr, Pro, His, or Thr. In some embodiments the nomacopan polypeptide is modified such that Asp149 of SEQ ID NO: 4 is replaced with Gly. This substitution at position 149 of SEQ ID NO: 4 (position 167 of SEQ ID NO: 2) may be made in combination with any of the other substitutions referred to herein.

In some modified nomacopan polypeptides which have reduced or absent C5-binding activity but which retain LTB-4-binding ability the nomacopan polypeptide can be modified such that at positions 114 to 124 of SEQ ID NO: 4 the following substitutions are present:

a. Met116 is replaced with Gln;

b. Leu117 is replaced with Ser;

c. Gly121 is replaced with Ala;

d. Leu122 is replaced with Asp;

e. Glu123 is replaced with Ala;

Ala44 in SEQ ID NO: 4 is replaced with Asn and Asp149 of SEQ ID NO: 4 is replaced with Gly149.

In preferred aspects of this embodiment the amino acid residues corresponding to positions 114 to 124 of SEQ ID NO: 4 are as set out in SEQ ID NO: 28.

In some modified nomacopan polypeptides which have reduced or absent C5-binding activity but which retain LTB-4-binding ability, the nomacopan polypeptide can be modified such that at positions 114 to 124 of SEQ ID NO: 4 the following substitutions are present:

a. Met116 is replaced with Gln;

b. Leu122 is replaced with Asp;

Ala44 in SEQ ID NO: 4 is replaced with Asn and Asp149 of SEQ ID NO: 4 is replaced with Gly149.

In preferred aspects of this embodiment the amino acid residues corresponding to positions 114 to 124 of SEQ ID NO: 4 are as set out in SEQ ID NO: 29.

In the various aspects and embodiments of this disclosure, the modified nomacopan polypeptides which have reduced or absent C5-binding activity but which retain LTB-4-binding ability differ from the unmodified nomacopan polypeptides in SEQ ID NO: 2 and SEQ ID NO: 4 by from 1 to 30 amino acids. Any modifications may be made to the nomacopan polypeptide in SEQ ID NO: 2 and SEQ ID NO: 4 provided that the resulting modified nomacopan polypeptide exhibits LK/E binding activity and reduced or absent C5 binding, compared to the unmodified nomacopan polypeptide.

In some embodiments the six cysteine amino acids at positions 6, 38, 100, 128, 129, 150 of SEQ ID NO: 4 are retained in the modified nomacopan polypeptides of the invention.

In some modified nomacopan polypeptides, Asn60 and Asn84 in SEQ ID NO: 4 are each replaced with Gln. This modification can be carried out by site directed mutagenesis to prevent N-linked hyperglycosylation when the polypeptide is expressed in yeast.

In some modified nomacopan polypeptides one or more of the following amino acids in SEQ ID NO: 4 are thought to be involved in binding to LTB4 and may therefore be retained in unmodified form: Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115. In some modified nomacopan polypeptides, at least five, ten or fifteen, or all of these amino acids are retained in unmodified form in the modified nomacopan polypeptides of the invention. In some modified nomacopan polypeptides which have reduced or absent C5-binding activity but which retain LTB-4-binding ability one or more of these amino acids may be conservatively substituted. In some modified nomacopan polypeptides which have reduced or absent C5-binding activity but which retain LTB-4-binding ability up to five, ten or fifteen, or all of these amino acids are conservatively substituted in the modified nomacopan polypeptides of the invention.

Amino acids at the following positions in SEQ ID NO: 4 are highly conserved between nomacopan and TSGP2 and TSGP3: 5, 6, 11, 13-15, 20-21, 24-27, 29-32, 35-41, 45, 47-48, 50, 52-60, 64, 66, 69-81, 83, 84, 86, 90-94, 97-104, 112-113, 115, 125-129, 132-139, 145, 148, and 150.

Amino acids at the following positions in SEQ ID NO: 4 are thought to be involved in binding to LTB4 and/or are highly conserved between nomacopan and TSGP2 and TSGP3: 5, 6, 11, 13-15, 18, 20-21, 24-27, 29-32, 35-41, 43, 45, 47-48, 50, 52-60, 64, 66, 67, 69-81, 83, 84, 86, 87, 89, 90-94, 97-104, 112-113, 115, 125-129, 132-139, 145, 148, and 150.

Amino acids at the following positions in SEQ ID NO: 4 are thought to be involved in binding to LTB4 and/or are highly conserved between nomacopan and TSGP2 and TSGP3: 5, 6, 11, 13-15, 18, 20-21, 24-25, 27, 30-32, 35-41, 43, 47-48, 50, 52-60, 64, 66, 67, 69-81, 83, 84, 86, 87, 89, 90-94, 98, 100, 102-104, 112-113, 115, 126, 128-129, 132-139, 145, 148, and 150.

In some modified nomacopan polypeptides which have reduced or absent C5-binding activity but which retain LTB-4-binding ability therefore the above amino acids are retained in unmodified form. In some embodiments, at least five, ten or fifteen, or all of these amino acids are retained in unmodified form in the modified nomacopan polypeptides of the invention. In some embodiments one or more of these amino acids may be conservatively substituted. In some embodiments up to five, ten or fifteen, twenty, twenty five, 30, 40, 50 or all of these amino acids are conservatively substituted in the modified nomacopan polypeptides of the invention

The modified nomacopan polypeptides referred to herein typically differ from SEQ ID NO: 2 or SEQ ID NO: 4 by from 1 to 30, preferably from 2 to 25, more preferably from 3 to 20, even more preferably from 4 to 15 amino acids. Typically the difference will be 5 to 12, or 6 to 10 amino acid changes. For example, from 1 to 30, or 2 to 25, 3 to 30, 4 to 15, 5 to 12, or 6 to 10 amino acid substitutions may be made in SEQ ID NO: 2 or SEQ ID NO: 4.

Modified nomacopan polypeptides which have the loop between beta H and alpha2 at amino acid positions 114 to 124 of SEQ ID NO: 4 (amino acid positions 132-142 of SEQ ID NO: 2) as set out in SEQ ID NO: 27 have 10 amino acid substitutions compared to SEQ ID NO: 4 as a result of the presence of this loop. In some embodiments, the modified nomacopan polypeptides referred to herein preferably therefore have 1-15, 2-10, 3-5, or up to 2, 3, 4 or 5 additional substitutions compared to SEQ ID NO: 4 beyond those that are set out in SEQ ID NO: 22 (e.g. in the loop of SEQ ID NO: 27).

Modified nomacopan polypeptides which have the loop between beta H and alpha2 at amino acid positions 114 to 124 of SEQ ID NO: 4 (amino acid positions 132-142 of SEQ ID NO: 2) as set out in SEQ ID NO: 28 have 5 amino acid substitutions compared to SEQ ID NO: 4 as a result of the presence of this loop. In some embodiments, the modified nomacopan polypeptides referred to herein preferably therefore have 1-20, 2-15, 3-10, or up to 2, 3, 4, 5, 6, 7, 8, 9, 10 additional substitutions compared to SEQ ID NO: 4 beyond those that are set out in SEQ ID NO: 23 (e.g. in the loop of SEQ ID NO: 28). The additional substitutions preferably include substitutions at position 44 and 149, as set out elsewhere herein.

Modified nomacopan polypeptides which have the loop between beta H and alpha2 at amino acid positions 114 to 124 of SEQ ID NO: 4 (amino acid positions 132-142 of SEQ ID NO: 2) as set out in SEQ ID NO: 29 have 2 amino acid substitutions compared to SEQ ID NO: 4 as a result of the presence of this loop. In some embodiments, the modified nomacopan polypeptides preferably therefore have 1-25, 2-12, 3-15, or up to 2, 3, 4, 5, 6, 7, 8, 9, 10, 11, 12, 13, 14, 15 additional substitutions compared to SEQ ID NO: 4 beyond those that are set out in SEQ ID NO: 24 (e.g. substitutions in the loop of SEQ ID NO: 29). The additional substitutions preferably include substitutions at position 44 and 149, as set out elsewhere herein.

Modified nomacopan polypeptides which have the substitution at position 44 of SEQ ID NO: 4 as set out elsewhere herein preferably have 1-25, 2-12, 3-15, or up to 2, 3 , 4, 5, 6, 7, 8, 9, 10, 11, 12, 13, 14, 15 additional substitutions compared to SEQ ID NO: 4.

Substitutions other than those explicitly referred to above are preferably conservative substitutions, for example according to the following Table. Amino acids in the same block in the second column and preferably in the same line in the third column may be substituted for each other:

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

Preferred modified nomacopan polypeptides which have reduced or absent C5-binding activity but which retain LTB-4-binding ability may comprise or consist of the amino acid sequences set out in one of SEQ ID NOs: 22, 23, 24, 25.

Examples of modified nomacopan polypeptides which have reduced or absent C5-binding activity but which retain LTB-4-binding ability include
1. A modified nomacopan polypeptide which exhibits leukotriene or hydroxyeicosanoid binding activity and reduced or absent C5 binding, said modified nomacopan polypeptide comprising SEQ ID NO: 4 in which from 1 to 30 amino acid substitutions are made, wherein
   (i) in positions 114 to 124 of SEQ ID NO: 4 one or more of the following substitutions (a)-(j) is made:
      a. Met114 is replaced with Gln, Asp, Asn, Glu, Arg, Lys, Gly, Ala, Pro, His, or Thr;
      b. Met116 is replaced with Gln, Asp, Asn, Glu, Arg, Lys, Gly, Ala, Pro, His, or Thr;
      c. Leu117 is replaced with Ser, Asp, Asn, Glu, Arg, Lys, Gly, Ala, or Pro;
      d. Asp118 is replaced with Asn, Gln, Arg, Lys, Gly, Ala, Leu, Ser, Ile, Phe, Tyr, Met Pro, His, or Thr;
      e. Ala119 is replaced with Gly, Asp, Asn, Glu, Arg, Lys, Leu, Ile, Phe, Tyr, Met, Pro, or His;
      f. Gly120 is replaced with Ser, Asp, Asn, Glu, Arg, Lys, Leu, Ile, Phe, Tyr, Met, Pro, or His;
      g. Gly121 is replaced with Ala, Asp, Asn, Glu, Arg, Lys, Leu, Ile, Phe, Tyr, Met, Pro, or His;
      h. Leu122 is replaced with Asp, Glu, Asn, Ala, Gln, Arg, Lys, Pro, or His;
      i. Glu123 is replaced with Asp, Ala, Gln, Asn, Arg, Lys, Gly, Leu, Ser, Ile, Phe, Tyr, Pro, His, or Thr;
      j. Val124 is replaced with Lys, Gln, Asn, Arg, Lys, Gly, Ala, Pro, His, or Thr; or/and wherein
   (ii) Ala44 in SEQ ID NO: 3 is replaced with Asn, Asp, Gln, Glu, Arg, Lys, Leu, Ile, Phe, Tyr, Met, Pro, or His;
   or a fragment thereof in which up to five amino acids are deleted from the N terminus of the modified nomacopan polypeptide.
2. A modified nomacopan polypeptide according to clause 1 wherein
   (i) in positions 114 to 124 of SEQ ID NO: 4 one or more of the following substitutions (a)-(j) is made:
      a. Met114 is replaced with Gln;
      b. Met116 is replaced with Gln;
      c. Leu117 is replaced with Ser;
      d. Asp118 is replaced with Asn;
      e. Ala119 is replaced with Gly;
      f. Gly120 is replaced with Ser;
      g. Gly121 is replaced with Ala;
      h. Leu122 is replaced with Asp;
      i. Glu123 is replaced with Asp, or Ala;
      j. Val124 is replaced with Lys; or/and wherein
   (ii) Ala44 in SEQ ID NO: 3 is replaced with Asn44;
   or a fragment thereof in which up to five amino acids are deleted from the N terminus of the modified nomacopan polypeptide.
3. A modified nomacopan polypeptide according to clause 1 or clause 2 or fragment thereof, wherein in positions 114 to 124 of SEQ ID NO: 4 one or more of the substitutions (a)-(j) is present.
4. A modified nomacopan polypeptide according to clause 3 or a fragment thereof, wherein two or more of the substitutions (a)-(j) are present.
5. A modified nomacopan polypeptide according to clause 4 or a fragment thereof, wherein five or more of the substitutions (a)-(j) are present.
6. A modified nomacopan polypeptide according to clause 5 or a fragment thereof, wherein each of the substitutions (a)-(j) is present, optionally wherein Trp 115 is not substituted.
7. A modified nomacopan polypeptide according to clause 5 or a fragment thereof, wherein each of the substitutions (a)-(j) as defined in clause 2 is present, optionally wherein Trp 115 is not substituted.
8. The modified polypeptide according to clause 7 or a fragment thereof, wherein Glu123 is replaced with Asp.
9. A modified nomacopan polypeptide according to any one of clauses 1 to 8, or a fragment thereof which has a loop sequence between amino acid positions 114 to 124 of SEQ ID NO:4 as set out in SEQ ID NO:27 and which has 1-15 additional substitutions compared to SEQ ID NO:4 beyond those that are set out in SEQ ID NO:22.
10. The modified nomacopan polypeptide according to clause 9, or a fragment thereof which has 2-10 additional substitutions compared to SEQ ID NO:27 beyond those that are set out in SEQ ID NO:22.
11. The modified nomacopan polypeptide according to clause 9 or 10, or a fragment thereof which has 3-5 additional substitutions compared to SEQ ID NO:27 beyond those that are set out in SEQ ID NO:22.
12. The modified nomacopan polypeptide according to any one of clauses 1 to 8 which consists of or comprises SEQ ID NO:22.
13. A modified nomacopan polypeptide according to any one of clauses 1 to 5, or a fragment thereof wherein:
   a. Met116 is replaced with Gln, Asp, Asn, Glu, Arg, Lys, Gly, Ala, Pro, His, or Thr, preferably Gln;
   b. Leu117 is replaced with Ser, Asp, Asn, Glu, Arg, Lys, Gly, Ala, or Pro, preferably Ser;
   c. Gly121 is replaced with Ala, Asp, Asn, Glu, Arg, Lys, Leu, Ile, Phe, Tyr, Met, Pro, or His, preferably Ala;
   d. Leu122 is replaced with Asp, Glu, Asn, Ala, Gln, Arg, Lys, Pro, or His, preferably Asp; and
   e. Glu123 is replaced with Asp, Ala, Gln, Asn, Arg, Lys, Gly, Leu, Ser, Ile, Phe, Tyr, Pro, His, or Thr, preferably Ala or Asp.
14. A modified nomacopan polypeptide according to clause 13, or a fragment thereof, wherein in positions 114 to 124 of SEQ ID NO: 4:
   a. Met116 is replaced with Gln;
   b. Leu117 is replaced with Ser;
   c. Gly121 is replaced with Ala;
   d. Leu122 is replaced with Asp; and
   e. Glu123 is replaced with Ala.
15. A modified nomacopan polypeptide according to clause 13 or clause 14, or a fragment thereof, wherein Trp 115 is not substituted.
16. A modified nomacopan polypeptide according to clause 13, 14 or 15, or a fragment thereof, wherein Met114, Trp 115, Asp118, Ala119, Gly120 and Val124 are not substituted.
17. A modified nomacopan polypeptide according to any one of clauses 1 to 5 or 13 to 16, or a fragment thereof which has a loop sequence between amino acid positions 114 to 124 of SEQ ID NO:4 as set out in SEQ ID NO:28 and which has 1-20 additional substitutions compared to SEQ ID NO:4 beyond those that are set out in SEQ ID NO:23.
18. The modified nomacopan polypeptide according to clause 17, or a fragment thereof which has 2-15 additional substitutions compared to SEQ ID NO:4 beyond those that are set out in SEQ ID NO:23.
19. The modified nomacopan polypeptide according to clause 17 or 18, or a fragment thereof which has 3-10 additional substitutions compared to SEQ ID NO:4 beyond those that are set out in SEQ ID NO:23.
20. The modified nomacopan polypeptide according to any one of clauses 1 to 5 or 13 to 16 which consists of or comprises SEQ ID NO:23.
21. A modified nomacopan polypeptide according to any one of clauses 1 to 4, or a fragment thereof, wherein:
   a. Met116 is replaced with Gln, Asp, Asn, Glu, Arg, Lys, Gly, Ala, Pro, His, or Thr, preferably Gln;
   b. Leu122 is replaced with Asp, Glu, Asn, Ala, Gln, Arg, Lys, Pro, or His, preferably Asp.
22. A modified nomacopan polypeptide according to clause 21 or a fragment thereof, wherein
   a. Met116 is replaced with Gln; and
   b. Leu122 is replaced with Asp.
23. A modified nomacopan polypeptide according to clause 21 or clause 22, or a fragment thereof, wherein Trp 115 is not substituted.
24. A modified nomacopan polypeptide according to clause 21, 22 or 23, or a fragment thereof, wherein Met114, Trp 115, Leu117, Asp118, Ala119, Gly120, Gly121, Glu123 and Val124 are not substituted.
25. A modified nomacopan polypeptide according to any one of clauses 1 to 4 or 21 to 24, or a fragment thereof which has a loop sequence between amino acid positions 114 to 124 of SEQ ID NO:4 as set out in SEQ ID NO:29 and which has 1-25 additional substitutions compared to SEQ ID NO:4 beyond those that are set out in SEQ ID NO:24.
26. The modified nomacopan polypeptide according to clause 25, or a fragment thereof which has 2-12 additional substitutions compared to SEQ ID NO:4 beyond those that are set out in SEQ ID NO:24.
27. The modified nomacopan polypeptide according to clause 25 or 26, or a fragment thereof which has 3-15 additional substitutions compared to SEQ ID NO:4 beyond those that are set out in SEQ ID NO:24.
28. The modified nomacopan polypeptide according to any one of clauses 1 to 4 or 21 to 24, which consists of or comprises SEQ ID NO:24.
29. A modified nomacopan polypeptide according to any one of clauses 1 to 11 or 13 to 28, or a fragment thereof, wherein Ala44 in SEQ ID NO: 4 is replaced with Asn, Asp, Gln, Glu, Arg, Lys, Leu, Ile, Phe, Tyr, Met, Pro, or His.
30. A modified nomacopan polypeptide according to clause 29, or a fragment thereof, wherein Ala44 in SEQ ID NO: 4 is replaced with Asn.
31. A modified nomacopan polypeptide according to any one of clauses 1 to 11 or 13 to 30, or a fragment thereof, wherein Asp149 in SEQ ID NO: 4 is replaced with Gly, Gln, Asn, Ala, Met, Arg, Lys, Leu, Ser, Ile, Phe, Tyr, Pro, His, or Thr.
32. A modified nomacopan polypeptide according to clause 30 or 31, wherein Ala44 in SEQ ID NO: 4 is replaced with Asn and Asp149 in SEQ ID NO: 4 is replaced with Gly.
33. A modified nomacopan polypeptide according to any one of the preceding clauses, or a fragment thereof, wherein the six cysteine amino acids at positions 6, 38, 100, 128, 129, 150 of SEQ ID NO: 4 are retained in unmodified form.
34. A modified nomacopan polypeptide according to any one of clauses 1 to 11, 13 to 19 or 21 to 27, wherein Asn60 and Asn84 are each replaced with Gln.
35. A modified nomacopan polypeptide according to any one of the preceding clauses, or a fragment thereof, wherein one or more of the following amino acids is not substituted: Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115.
36. A modified nomacopan polypeptide according to clause 35, or a fragment thereof, wherein all of the following amino acids are not substituted: Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115.
37. A modified nomacopan polypeptide according to any one of clauses 1 to 36, or a fragment thereof wherein:
   a. none of amino acids 5, 6, 11, 13-15, 20-21, 24-27, 29-32, 35-41, 45, 47-48, 50, 52-60, 64, 66, 69-81, 83, 84, 86, 90-94, 97-104, 112-113, 115, 125-129, 132-139, 145, 148, and 150 in SEQ ID NO:4 are substituted; or
   b. none of amino acids 5, 6, 11, 13-15, 18, 20-21, 24-27, 29-32, 35-41, 43, 45, 47-48, 50, 52-60, 64, 66, 67, 69-81, 83, 84, 86, 87, 89, 90-94, 97-104, 112-113, 115, 125-129, 132-139, 145, 148, and 150 in SEQ ID NO:4 are substituted; or
   c. none of amino acids 5, 6, 11, 13-15, 18, 20-21, 24-25, 27, 30-32, 35-41, 43, 47-48, 50, 52-60, 64, 66, 67, 69-81, 83, 84, 86, 87, 89, 90-94, 98, 100, 102-104, 112-113, 115, 126, 128-129, 132-139, 145, 148, and 150 in SEQ ID NO:4 are substituted.
38. A modified nomacopan polypeptide according to clause 1 or clause 2 which comprises or consists of the sequence SEQ ID NO: 25.
39. A modified nomacopan polypeptide according to any one of the preceding clauses or a fragment thereof which binds to LTB4.

Fragments

Functional equivalents of nomacopan include fragments of the nomacopan protein providing that such fragments retain the ability to (i) bind LTB4 and/or (ii) C5 (e.g. wild-type C5 and/or C5 from subjects with a C5 polymorphism that renders treatment by eculizumab ineffective, or reduce the efficacy of treatment with eculizumab). Preferably the functional fragment has property (i) and (ii). In other preferred embodiments the functional fragment has property (i) but reduced or absent C5 binding.

Fragments may include, for example, polypeptides derived from the nomacopan protein sequence (or homologue) which are less than 150 amino acids, less than 145 amino acids, provided that these fragments retain the ability to bind to LTB4 and optionally also C5.

Fragments may include, for example, polypeptides derived from the nomacopan protein sequence (or homologue) which are at least 150 amino acids, at least 145 amino acids, provided that these fragments retain the ability to bind to LTB4 and optionally also C5.

Any functional equivalent or fragment thereof preferably retains the pattern of cysteine residues that is found in nomacopan. For example, said functional equivalent comprises six cysteine residues that are spaced relative to each other at a distance of 32 amino acids apart, 62 amino acids apart, 28 amino acids apart, 1 amino acid apart and 21 amino acids apart as arranged from the amino terminus to the carboxyl terminus of the sequence according to amino acids 1 to 168 of the amino acid sequence in FIG. 2 (SEQ ID NO:2). Exemplary fragments of nomacopan protein are disclosed in SEQ ID NO: 4, SEQ ID NO: 6, SEQ ID NO: 8, SEQ ID NO: 10, SEQ ID NO: 12, SEQ ID NO: 14. The DNA encoding the corresponding fragments are disclosed in SEQ ID NO: 3, SEQ ID NO: 5, SEQ ID NO: 7, SEQ ID NO: 9, SEQ ID NO: 11, SEQ ID NO: 13.

Included as such fragments are not only fragments of the O. moubata nomacopan protein that is explicitly identified herein in FIG. 2, but also fragments of homologues (e.g. variants) of this protein, as described above. Such fragments of homologues will typically possess greater than 60% identity with fragments of the nomacopan protein sequence in FIG. 2, although more preferred fragments of homologues will display degrees of identity of greater than 70%, 80%, 90%, 95%, 98% or 99%, respectively with fragments of the nomacopan protein sequence in FIG. 2. Preferably such fragments will retain the cysteine spacing referred to above. Fragments with improved properties may, of course, be rationally designed by the systematic mutation or fragmentation of the wild type sequence followed by appropriate activity assays. Fragments may exhibit similar or greater affinity for LTB4 as nomacopan and optionally also similar or greater affinity for C5 as nomacopan. These fragments may be of a size described above for fragments of the nomacopan protein.

As discussed above, nomacopan-type proteins preferably bind to LTB4 and optionally also C5.

Conservative Substitutions

Any substitutions are preferably conservative substitutions, for example according to the following Table. Amino acids in the same block in the second column and preferably in the same line in the third column may be substituted for each other:

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

Fusion Proteins

A functional equivalent used according to the invention may be a fusion protein, obtained, for example, by cloning a polynucleotide encoding the nomacopan protein or a functionally equivalent in frame to the coding sequences for a heterologous protein sequence.

The term “heterologous”, when used herein, is intended to designate any polypeptide other than the nomacopan protein or its functional equivalent. Example of heterologous sequences, that can be comprised in the soluble fusion proteins either at N- or at C-terminus, are the following: extracellular domains of membrane-bound protein, immunoglobulin constant regions (Fc region), PAS or XTEN or similar unstructured polypeptides, multimerization domains, domains of extracellular proteins, signal sequences, export sequences, or sequences allowing purification by affinity chromatography. Many of these heterologous sequences are commercially available in expression plasmids since these sequences are commonly included in the fusion proteins in order to provide additional properties without significantly impairing the specific biological activity of the protein fused to them [51]. Examples of such additional properties are a longer lasting half-life in body fluids (e.g. resulting from the addition of an Fc region or PASylation [52]), the extracellular localization, or an easier purification procedure as allowed by a tag such as a histidine, GST, FLAG, avidin or HA tag. Fusion proteins may additionally contain linker sequences (e.g. 1-50 amino acids in length, such that the components are separated by this linker.

Fusion proteins are thus examples of proteins comprising a nomacopan-like protein, and include by way of specific example a protein comprising a PAS sequence and a nomacopan-type protein sequence. PAS sequences are described e.g. in [52], and EP2173890, with a PASylated nomacopan molecule being described in Kuhn et al [53]. PASylation® describes the genetic fusion of a protein with conformationally disordered polypeptide sequences composed of the amino acids Pro, Ala, and/or Ser. This is a technology developed by XL Protein (http://xl-protein.com/) and provides a simple way to attach a solvated random chain with large hydrodynamic volume to the protein to which it is fused. The polypeptide sequence adopts a random coil structure. The apparent molecular weight of the resulting fusion protein is thus much larger than the actual molecular weight of the fusion protein. This greatly reduces clearance rates by kidney filtration in biological systems. Appropriate PAS sequences are described in EP2173890, as well as [52]. Any suitable PAS sequence may be used in the fusion protein. Examples include an amino acid sequence consisting of at least about 100 amino acid residues forming a random coil conformation and consisting of or consisting essentially of alanine, serine and proline residues (or consisting of or consisting essentially of proline and alanine residues). This may comprise a plurality of amino acid repeats, wherein said repeats consist of or consist essentially of Ala, Ser, and Pro residues (or proline and alanine residues) and wherein no more than 6 consecutive amino acid residues are identical. Proline residues may constitute more than 4% and less than 40% of the amino acids of the sequence. The sequence may comprise an amino acid sequence selected from:

(SEQ ID NO: 15)
ASPAAPAPASPAAPAPSAPA;
(SEQ ID NO: 16)
AAPASPAPAAPSAPAPAAPS;
(SEQ ID NO: 17)
APSSPSPSAPSSPSPASPSS,
(SEQ ID NO: 18)
SAPSSPSPSAPSSPSPASPS,
(SEQ ID NO: 19)
SSPSAPSPSSPASPSPSSPA,
(SEQ ID NO: 20)
AASPAAPSAPPAAASPAAPSAPPA
and
(SEQ ID NO: 21)
ASAAAPAAASAAASAPSAAA

or circular permuted versions or multimers of these sequences as a whole or parts of these sequences. There may, for example be 5-40, 10-30, 15-25, 18-20 preferably 20-30 or 30 copies of one of the repeats present in the PAS sequence, i.e. one of SEQ ID NOs 15-21, preferably SEQ ID NO:15. Preferably the PAS sequence comprises or consists of 30 copies of SEQ ID NO:15. Preferably the PAS sequence is fused to the N terminus of the nomacopan-type protein (directly or via a linker sequence).

In certain preferred embodiments the nomacopan-type protein may comprise or consist of amino acids 19-168 of SEQ ID NO:2 (e.g. the fusion protein comprises (a) a PAS sequence consisting of 30 copies of SEQ ID NO:15 and (b) amino acids 19-168 of SEQ ID NO:2, wherein (a) is fused to the N terminus of (b) directly or via a linker sequence). An exemplary sequence is provided in FIG. 2D and SEQ ID NO:30.

In another preferred embodiment the nomacopan-type protein may comprise or consist of SEQ ID NO:22, 23, 24 or 25 (e.g. the fusion protein comprises (a) a PAS sequence consisting of 30 copies of SEQ ID NO:15 and (b) SEQ ID NO: 22, 23, 24 or 25, wherein (a) is fused to the N terminus of (b) directly or via a linker sequence). A preferred version will contain SEQ ID NO:22. An exemplary sequence is provided in FIG. 2E and SEQ ID NO:31. Fusion proteins may additionally contain linker sequences (e.g. 1-50, 2-30, 3-20, 5-10, 2-4, 3-5, 1, 2, 3, 4, 5, 6, 7, 8, 9, 10 or up to 1, 2, 3, 4, 5, 6, 7, 8, 9, 10 amino acids in length), such that the components are separated by this linker. In one embodiment the linker sequence can be a single alanine residue.

In the present “PAS-nomacopan” is intended to refer to a functional equivalent of nomacopan that is PASylated, e.g. as described above and PAS-L-nomacopan is intended to refer to a functional equivalent of nomacopan that binds LTB4 but not C5 and which is PASylated . The sequence of the tested PAS-nomacopan molecule in the Examples is set out in FIG. 2D and SEQ ID NO:30. The sequence of the tested PAS-L-nomacopan molecule in Examples is set out in FIG. 2E and SEQ ID NO:31. PAS-nomacopan and PAS-L-nomacopan have the advantage that they each have a longer half-life, which allows less frequent administration, which is more convenient for patients. PAS-nomacopan thus combines the advantages of nomacopan, in that it inhibits both the C5 and the LTB4 dependent pathways, yet can be administered less frequently than nomacopan thus providing an administration advantage. PAS-L-nomacopan thus combines the advantages of L-nomacopan, in that it inhibits both the the LTB4 dependent pathway without inhibiting the C5 dependent pathway, yet can be administered less frequently than L-nomacopan thus providing an administration advantage.

Preparation of Agents

The protein and functional equivalents thereof, may be prepared in recombinant form by expression in a host cell. Such expression methods are well known to those of skill in the art and are described in detail by [54] and [55]. Recombinant forms of the nomacopan protein and functional equivalents thereof are preferably unglycosylated. Preferably the host cell is E. coli.

The nomacopan protein and functional equivalents thereof, are preferably in isolated form, e.g. separated from at least one component of the host cell and/or cell growth media in which it was expressed. In some embodiments, the nomacopan protein or functional equivalent thereof is purified to at least 90%, 95%, or 99% purity as determined, for example, by electrophoresis or chromatography. The proteins and fragments of the present invention can also be prepared using conventional techniques of protein chemistry. For example, protein fragments may be prepared by chemical synthesis. Methods for the generation of fusion proteins are standard in the art and will be known to the skilled reader. For example, most general molecular biology, microbiology recombinant DNA technology and immunological techniques can be found in [54] or [56].

Agent as Nucleic Acid Molecule

According to a further embodiment of the invention, the agent may be a nucleic acid molecule encoding the nomacopan-type protein. For example, gene therapy may be employed to effect the endogenous production of the nomacopan-type protein by the relevant cells in the subject, either in vivo or ex vivo. Another approach is the administration of “naked DNA” in which the therapeutic gene is directly injected into the bloodstream or into muscle tissue.

Preferably, such a nucleic acid molecule comprises or consists of bases 55 to 507 of the nucleotide sequence in FIG. 2 (SEQ ID NO: 1). This nucleotide sequence encodes the nomacopan protein in FIG. 2 without the signal sequence. The first 54 bases of the nucleotide sequence in FIG. 2 encode the signal sequence which is not required for complement inhibitory activity or LTB4 binding activity. Alternatively, the nucleic acid molecule may comprise or consist of bases 1 to 507 of the nucleic acid sequence in FIG. 2, which encodes the protein with the signal sequence.

Modes of Administration

Nomacopan-type proteins may be administered directly to the eye or systemically. They are preferably administered directly onto the eye surface, e.g. topically (e.g. in the form of drops or ointments) or by direct introduction into the eye (e.g. direct administration within the boundary of the eye as defined by the sclera). Examples of direct administration within the boundary of the eye include intravitreal administration and suprachoroidal administration. Intravitreal administration (e.g. intravitreal injection) is well known in the art. Suprachoroidal administration may also be carried out.

Topical Administration

The inventors have observed that administration of nomacopan-type proteins topically to the eye affects experimental UAE, which is surprising as it was not previously thought that such a protein could traverse the cornea. As shown in Example 1, L-nomacopan, nomacopan and PAS-nomacopan all reduced clinical score to some degree in the UAE mouse model that was used. This is consistent with these molecules being able to traverse the cornea and carry out their effect within the eye.

The fact that topical treatment with nomacopan-type proteins has an effect on retinal proliferation diseases, i.e. diseases where the pathological effect is within the eye means that any of the nomacopan-type proteins can be used alone for the treatment of retinal proliferation diseases in certain embodiments. In certain embodiments of the invention, nomacopan-type proteins are administered topically e.g. daily, or 2-5, 3-4 times daily.

Administration Directly Into the Eye

It can be advantageous to introduce the agent of the invention directly into the eye, e.g direct administration within the boundary of the eye as defined by the sclera. In certain embodiments the agent of the invention is introduced intravitreally. In other embodiments the agent of the invention is introduced suprachoroidally.

In certain embodiments the agent of the invention is introduced intravitreally. Intravitreal administration is well known in the art. In certain embodiments of the invention intravitreal administration is performed about once every 4-6, 4-8, 4-10 weeks.

In other embodiments the agent of the invention is introduced suprachoroidally. The suprachoroidal space (SCS) is a potential space between the sclera and choroid that traverses the circumference of the posterior segment of the eye. Introducing agents into the SCS targets the choroid, retinal pigment epithelium, and retina with high bioavailability, while maintaining low levels elsewhere in the eye. Indeed, phase III clinical trials are investigating the safety and efficacy of SCS drug delivery. Agents can be administered to the SCS e.g. with a hypodermic needle, microneedle, micro-stent [57]. In certain embodiments of the invention suprachoroidal administration is performed about once every 4-6, 4-8, 4-10 weeks.

Topical Administration in Combination With Administration Directly Into the Eye

Nomacopan-type proteins may also be used in combination with a second agent, as discussed elsewhere herein. In certain embodiments the second agent is introduced directly into the eye. The advantage of this is that it may reduce the frequency with which administration of said second agent, directly into the eye, needs to be carried out. Administration directly into the eye, although generally safe, has certain risks of complications (e.g. infections, blockage of the main artery into the eye, retinal detachment, bleeding in the eye and damage to the lens in the eye). It is furthermore a medical procedure which requires patients to attend hospital. For treating retinal proliferation diseases such administrations can be carried out e.g. once every 1 to 2 months. It would therefore be advantageous to reduce the frequency with which these administrations are carried out.

Therefore, in certain embodiments the method of treating or preventing a retinal proliferation disease in a subject comprises (i) topical administration of a nomacopan-type protein as defined herein and (ii) administration of a second retinal proliferation treatment directly into the eye in said subject. The second retinal proliferation treatment may be as defined elsewhere herein, or may itself be a nomacopan-type protein (e.g. it may be the same nomacopan-type protein as the nomacopan-type protein that is administered topically or it may be a second nomacopan-type protein).

Examples of a second retinal proliferation treatment include an anti-VEGF agent (e.g. an anti-VEGF-A antibody or fragment thereof such as bevacizumab (Avastin), ranibizumab (Lucentis), an anti-VEGF aptamer (such as pegaptanib (Macugen)), or another VEGF antagonist such as aflibercept (Eylea, a recombinant fusion protein consisting of vascular endothelial growth factor (VEGF)-binding portions from the extracellular domains of human VEGF receptors 1 and 2, that are fused to the Fc portion of the human IgG1 immunoglobulin), a steroid (e.g. corticosteroid), an immunomodulatory therapy (IMT) drug (e.g. methotrexate, azathioprine or mycophenolate), a biologic response modifier (BRM) drug (an anti-TNFalpha agent, for example an antibody or fragment thereof that bind TNFalpha, such as infliximab or adalimumab). Preferably said second agent is an anti-VEGF agent.

In embodiments where a nomacopan-type protein is administered topically and a second nomacopan-type protein is administered directly into the eye, the agent which is administered topically does not have to be a long acting protein (e.g. a fusion protein which contains sequences that are designed to reduce clearance time). It therefore may e.g. be an agent which is a protein comprising amino acids 19 to 168 of the amino acid sequence in FIG. 2 (SEQ ID NO: 2) or a functional equivalent of this protein wherein the protein is up to 150, 151, 152, 153, 154, 155, 156, 157, 158, 159, 160 amino acids (preferably up to 150 amino acids) in length. For example it may be an agent which is a protein comprising amino acids 19 to 168 of the amino acid sequence in FIG. 2 (SEQ ID NO:2) or a functional equivalent thereof, wherein if said protein is a functional equivalent it is not a fusion protein (e.g. it may be a functional equivalent that is a homologue of said protein, a fragment of said protein, or a fragment of a homologue where the homologue is as defined elsewhere herein). In these embodiments the agent that is administered directly into the eye may be a functional equivalent that is a fusion protein as defined elsewhere herein. By way of example the agent that is administered topically may be a protein consisting of amino acids 19 to 168 of the amino acid sequence in FIG. 2 (SEQ ID NO:2), SEQ ID NO:22, 23, 24, 25, preferably amino acids 19-168 of SEQ ID NO:2 or SEQ ID NO:22 and/or the agent that is administered directly into the eye is a fusion protein comprising (i) amino acids 19 to 168 of the amino acid sequence in FIG. 2 (SEQ ID NO:2), SEQ ID NO:22, 23, 24, 25, preferably amino acids 19-168 of SEQ ID NO:2 or SEQ ID NO:22 and (ii) a PAS sequence as defined herein, e.g. a sequence consisting of 30 copies of SEQ ID NO:15.

In other embodiments one agent which is a protein comprising amino acids 19 to 168 of the amino acid sequence in FIG. 2 (SEQ ID NO: 2) or a functional equivalent of this protein is administered topically and a second retinal proliferative disease treatment is administered directly into the eye. The second retinal proliferative disease treatment may for example an anti-VEGF agent (e.g. an anti-VEGF-A antibody or fragment thereof such as bevacizumab (Avastin), ranibizumab (Lucentis), an anti-VEGF aptamer (such as pegaptanib (Macugen)), or another VEGF antagonist such as aflibercept (Eylea, a recombinant fusion protein consisting of vascular endothelial growth factor (VEGF)-binding portions from the extracellular domains of human VEGF receptors 1 and 2, that are fused to the Fc portion of the human IgG1 immunoglobulin, a steroid (e.g. corticosteroid), an immunomodulatory therapy (IMT) drug (e.g. methotrexate, azathioprine or mycophenolate), a biologic response modifier (BRM) drug (an anti-TNFalpha agent, for example an antibody or fragment thereof that bind TNFalpha, such as infliximab or adalimumab). Preferably said second agent is an anti-VEGF agent. Preferably the agent that is administered topically is a protein consisting of amino acids 19 to 168 of the amino acid sequence in FIG. 2 (SEQ ID NO:2), SEQ ID NO:22, 23, 24, 25, preferably amino acids 19-168 of SEQ ID NO:2 or SEQ ID NO:22.

Systemic Administration

In other embodiments the nomacopan-type protein or agent is administered systemically, e.g. subcutaneously.

Therapeutically or Prophylactically Effective Amount

The agent is administered in a therapeutically or prophylactically effective amount. The term “therapeutically effective amount” refers to the amount of agent needed to treat the relevant condition, e.g. retinal proliferative disease. In this context, “treating” includes reducing the severity of the disorder.

The term “prophylactically effective amount” used herein refers to the amount of agent needed to prevent the relevant condition, e.g. retinal proliferative disease. In this context, “preventing” includes reducing the severity of the disorder, e.g. if the presence of the disorder is not detected before the administration of the agent is commenced. Reducing the severity of the disorder could be, for example, improving visual acuity.

The reduction or improvement is relative to the outcome without administration or the agent as described herein. The outcomes are assessed according to the standard criteria used to assess such patients. To the extent that this can be quantitated, there is a reduction or improvement of at least 10, 20, 30, 40, 50, 60, 70, 80, 90, 100% in the relative criteria.

For systemic administration, the dose, calculated on the basis of the nomacopan molecule is from 0.1 mg/kg/day to 10 mg/kg/day (mass of drug compared to mass of patient), e.g. 0.2-5, 0.25-2, or 0.1-1 mg/kg/day. As fusion proteins (e.g. as discussed herein) are larger than the nomacopan molecule an equivalent molar amount could be used for such proteins. Thus for a functional equivalent of nomacopan, an equivalent molar amount of the dose referred to above can be used. For example for a fusion protein comprising nomacopan and a PAS 30 portion of about 600 amino acids, or a PAS portion as defined herein, e.g. PAS-nomacopan) an equivalent molar amount of 0.1 mg/kg/day is 0.4 mg/kg/day, so the dose could be 0.4 mg/kg/day to 40 mg/kg/day (mass of drug compared to mass of patient), e.g. 0.8-20, 1-8, or 0.4-4 mg/kg/day. Alternatively, and to account for the longer half-life of these fusion proteins, greater amounts can be given per dose, and the dose administered less often, e.g. 40 mg-2 g, 50 mg-1.5 g, 75 mg-1 g, over the course of one week, e.g. with administration being e.g. one or twice per week.

The therapeutically or prophylactically effective amount can additionally be defined in terms of the inhibition of terminal complement, for example, an amount that means that terminal complement activity (TCA) is reduced by at least 10, 20, 30, 40, 50, 60, 70, 80, 90, 91, 92, 93, 94, 95, 96, 97, 98, 99, 100%, compared to terminal complement activity in the absence of treatment. Dose and frequency may be adjusted in order to maintain terminal complement activity at the desired level, which may be, for example 10% or less, for example 9, 8, 7, 6, 5, 4, 3, 2, 1% or less compared to terminal complement activity in the absence of treatment.

The therapeutically or prophylactically effective amount can additionally be defined in terms of the reduction of LTB4 levels in plasma, for example, an amount that means that the LTB4 level in plasma is reduced by at least 10, 20, 30, 40, 50, 60, 70, 80, 90, 91, 92, 93, 94, 95, 96, 97, 98, 99, 100%, compared to the LTB4 level in plasma in the absence of treatment or which causes LTB4 levels to be within a certain range of the normal levels (e.g. 90-110% of normal, 85-115% of normal). Dose and frequency may be adjusted in order to maintain the LTB4 level in plasma at the desired level, which may be, for example 90%, 80%, 70%, 60%, 50%, 40%, 30%, 20%, 10% or less, for example 9, 8, 7, 6, 5, 4, 3, 2, 1% or less compared to the LTB4 level in plasma in the absence of treatment or which is within a certain range of the normal levels (e.g. 90-110% of normal, 85-115% of normal). LTB4 levels may be determined by routine methods (e.g. immunoassays, see e.g. the commercially available R&D Systems assay based on a sequential competitive binding technique [58]).

Where a dose is given, this relates to a dose of the agent which is a protein or functional equivalent thereof. Appropriate doses for an agent which is a nucleic acid molecule may be used to give rise to these levels. Doses may vary to account for the presence of non-active protein present (e.g. PAS-nomacopan with a 600 amino acid PAS portion has approximately 4× the molecular weight of nomacopan so an equivalent molar amount would be approximately four times the amount of nomacopan). An equivalent molar amount of any dose provided for nomacopan may be used for any nomacopan functional equivalent thereof which contains additional sequence. The equivalent molar amount can be calculated using routine methods.

Terminal complement activity can be measured by standard assays known in the art, e.g. using the Quidel CH₅₀ haemolysis assay and the sheep red blood cell lytic CH50 assay.

The frequency with which the dose needs to be administered will depend on the half-life of the agent involved. The nomacopan protein or a functional equivalent thereof, may be administered e.g. on a twice daily basis, daily basis, or every two, three, four days, five, six, or seven, days or more e.g. twice daily or on a daily basis). Extended half-life versions, e.g. PASylated nomacopan molecules could be administered less frequently (e.g. every two, three, four days, five, six, seven, 10, 15 or 20 days or more, e.g. once daily or every two or more days, or every week)

The exact dosage and the frequency of doses may also be dependent on the patient's status at the time of administration. Factors that may be taken into consideration when determining dosage include the need for treatment or prophylaxis, the severity of the disease state in the patient, the general health of the patient, the age, weight, gender, diet, time and frequency of administration, drug combinations, reaction sensitivities and the patient's tolerance or response to therapy. The precise amount can be determined by routine experimentation, but may ultimately lie with the judgement of the clinician.

The dosage regimen may also take the form of an initial “ablating regimen” followed by one or more subsequent doses (e.g. maintenance dose). In general, the ablating regimen will be greater than the subsequent dose(s). By way of example for nomacopan this may be an ablating regimen of 0.6-1.2 mg/kg, then 0.3-0.6 mg/kg 8-18, 10-14, or 11-13 hours (e.g. about 12 hours) later, followed by a maintenance dose of 0.45-0.9 mg/kg, which may be administered e.g. once daily.

For PASylated versions (e.g. PAS-nomacopan, e.g. as described elsewhere herein a suitable regimen may be an ablating regimen of 6-12 mg/kg (e.g. 600 mg), then 6-12 mg/kg (e.g. 600 mg) 3-10, 4-8, 5-7, e.g. about 7 days later, followed by a maintenance dose of 4-8 mg/kg (e.g. 400 mg), which may be administered e.g. once daily.

The ablating dose or doses may be at least 1.5, 2, or 5 times greater than the maintenance dose. The ablating dose may be administered as a single dose, or as one or more doses in a particular time frame (e.g. two doses). Typically, the loading dose will be 1, 2, 3, 4 or 5 doses administered in a single 24 hour period (or a single week for an extended half-life version). The maintenance dose may be a lower dose that is repeated at regular intervals. The maintenance dose may be repeated at intervals, such as every 12, 24, or 48 hours (or every week, or every two weeks for an extended half-life version). The precise regimen can be determined by routine experimentation, but may ultimately lie with the judgement of the clinician. The maintenance dose may be at least 20, 30, 40, 50, 60, 70, 80, 90 or 100% of the initial ablating dose, or up to 20, 30, 40, 50, 60, 70, 80, 90 or 100% of the initial ablating dose.

In a further embodiment the same dose is used throughout the course of treatment (e.g. daily or twice daily or weekly).

Preferably, the topical dose of the nomacopan-type protein is between 50 and 500 μg per dose, more preferably between 100 and 400 μg per dose and most preferably between 200 and 300 μg per dose. Alternatively the dose may be 500-2000, 600-1500, 700-1250 or about 1250 μg per dose. For a fusion protein the dose may be selected to give an equivalent molar amount of the active agent, which e.g. for a PAS-nomacopan is approximately 4 times the amount per dose (e.g. between 400 and 1600 μg per dose and most preferably between 800 and 1200 μg per dose). Alternatively the dose may be 2000-8000, 2400-6000, 2800-5000 or about 5000 μg per dose.

In applying a composition topically it is usually indicated that a certain number of drops or certain length of an ointment be applied to an eye. It will be a matter of routine to the skilled person to adjust the concentration of the nomacopan-type protein in any composition to ensure that the correct dose is administered when the application instructions are followed. For example one drop is usually about 40 μl, and so one drop of a solution containing 0.5% w/v nomacopan or L-nomacopan will contain 200 μg of nomacopan or L-nomacopan. Likewise, one drop of a solution containing 2% w/v of PAS-nomacopan or PAS-L-nomacopan will contain 800 μg of PAS-nomacopan or PAS-L-nomacopan. Since PAS-nomacopan or PAS-L-nomacopan has a molecular weight approximately 4 times that of nomacopan or L-nomacopan equal volumes of a solution containing 2% w/v of PAS-nomacopan or PAS-L-nomacopan a solution containing 0.5% w/v nomacopan or L-nomacopan give equivalent molar amounts of the nomacopan portions of the proteins. Similar doses of nomacopan-type molecules may be used for administration directly into the eye. In applying a composition directly into the eye, a volume of e.g. 10-50 μl may be used. It will be a matter of routine to the skilled person to adjust the concentration of the nomacopan-type protein in any composition to ensure that the correct dose is administered. For example if 50 μl of a solution containing 0.5% w/v nomacopan or L-nomacopan is used, this will contain 250 μg of nomacopan or L-nomacopan. Likewise, one drop of a solution containing 2% w/v of PAS-nomacopan or PAS-L-nomacopan will contain 1000 μg of PAS-nomacopan or PAS-L-nomacopan.

The composition for topical administration or administration directly into the eye may also be defined in terms of its nomacopan-type protein concentration e.g. 0.1-20% w/v, 0.2-15, 0.25-10, 0.5-5% w/v (e.g. 0.4-80, 0.8-60, 1-40, 2-20% w/v for a PASylated version thereof).

Pharmaceutical Formulations

The agent will generally be administered in conjunction with or in a pharmaceutically acceptable carrier. The term “pharmaceutically acceptable carrier”, in general will be a liquid or but may include other agents provided that the carrier does not itself induce toxicity effects or cause the production of antibodies that are harmful to the individual receiving the pharmaceutical composition. Pharmaceutically acceptable carriers may e.g. contain liquids such as water, saline, glycerol, ethanol or auxiliary substances such as wetting or emulsifying agents, pH buffering substances and the like. The pharmaceutical carrier employed will thus vary depending on the route of administration. A thorough discussion of pharmaceutically acceptable carriers is available in [59]. In a preferred embodiment the agent is administered in an optically acceptable composition which may be a liquid, e.g. in a solution in water or PBS.

The agent may optionally be delivered using colloidal delivery systems (e.g. liposomes, nanoparticles or microparticles (e.g. as discussed in [60])). Advantages of these carrier systems include protection of sensitive proteins, prolonged release, reduction of administration frequency, patient compliance and controlled plasma levels.

Liposomes (e.g. comprising phospholipids of synthetic and/or natural origin) may e.g. be 20 nm 100 or 200 micrometers, e.g. small unilamellar vesicles (25-50 nm), large unilamellar vesicles (100-200 nm), giant unilamellar vesicles (1-2 μm) or multilamellar vesicles (MLV; 1 μm-2 μm).

Nanoparticles (colloidal carriers with size ranging from 10 to 1000 nm) can be fabricated from lipids, polymers or metal. Polymeric nanoparticles may be made from natural or synthetic polymers (e.g. chitosan, alginate, PCL, polylactic acid (PLA), poly (glycolide), PLGA and may be generated as nanospheres (molecules are uniformly distributed into polymeric matrix) or nanocapsules (carrying drug molecules confined within a polymeric membrane).

Microparticles e.g. made of starch, alginate, collagen, poly (lactide-co-glycolide) (PLGA), polycaprolactones (PCL) can also be used.

Hydrogels may alternatively or additionally be present.

For larger molecular weight molecules, e.g. fusion proteins additional excipients such as hyaluronidase may also be used, e.g. to allow administration of larger volumes (e.g. 2-20ml).

Duration of Treatment

Preferably the course of treatment is continued for at least 1, 2, 3, 4, 5 or 6 weeks, or at least 1, 2, 3, 4, 5 or 6 months or at least 1, 2, 3, 4, 5 or 6 years. The course of treatment is preferably continued at least until the subject's symptoms have reduced. The course of treatment may thus be administration of the agent (e.g. daily, every other day or weekly) for at least 1, 2, 3, 4, 5, 6, 7, 8, 9, 10, 15, 20, 25, 30, 35, 40 weeks.

### BRIEF DESCRIPTION OF FIGURES

FIG. 1A: Schematic diagram of classical and alternative pathways of complement activation. Anaphylatoxins enclosed in starbursts.

FIG. 1B: Schematic diagram of the eicosanoid pathway.

FIG. 2A: Primary sequence of nomacopan. Signal sequence underlined. Cysteine residues in bold type. Nucleotide and amino acid number indicated at right.

FIG. 2B: Examples of nomacopan variants

FIG. 2C: Examples of nomacopan variants that bind LTB4 but which show reduced or absent C5 binding.

FIGS. 2D and E: Examplary PAS-nomacopan (D) and PAS-L-nomacopan sequences (E).

FIG. 3: Shows results of fundus examination for individual eyes (n=12) after 7 days of topical treatment on day 21 post immunization. Bars indicate SD or SEM and two tailed nonparametric t test has been applied.

FIG. 4: Shows BLT1 (LTB4 receptor) and C5a receptors on cells within the retinas of mice used in an experimental autoimmune uveitis (EAU) model. The images show infiltrating cells captured in vitreous. Nuclei are shown in blue, BLT1 in green and the C5a receptor in red. There are cells expressing both receptors (yellow). Cells indicated by arrows.

FIG. 5: Shows the clinical score in EAU mice pre treatment at 15 days post immunization and following intravitreal administration of nomacopan-type proteins on day 18 post immunization. Fundoscopic evaluation of disease was performed on day 15 (pre-treatment) and 22 (post treatment) for individual eyes (n=16). On day 15 and 18 EAU mice were treated intravitreally with 1-2 ul of indicated compounds. Representative fundus and corresponding histopathology images are shown, together with a graphical summary showing histology score from treated mice (n=3 in each group) (nomacopan Bars indicate SD or SEM and two tailed nonparametric t test has been applied.

FIG. 6: Shows the results of flow cytometric analysis of cells. (A-B) RORgt/T.bet+ expression in CD4+ infiltrating cells in the EAU mice (RORgt expression on the y axis and Tbet expression on the x axis). (C-D) RORgt+ expression alone in CD4+ cells (RORgt expression on the x axis, FSC-H on y axis). (E-F) T.bet expression alone in CD4+ cells (T.bet expression on the x axis, FSC-H on y axis)). (G-H) IL-17A expression in CD4+ cells (IL-17A expression on the x axis, FSC-H on y axis). In each of FIGS. 6b, 6d, 6f and 6h, the top row of results on each page is for saline treated EAU mice, the second is for nomacopan treated mice, the third is for PAS-nomacopan treated mice, the fourth is for PAS-L-nomacopan treated mice and the fifth is for dexamethasone treated mice. FIGS. 6A, C, E, G show graphical summaries of the respective expression levels. Bars indicate SEM. The graph shows means of 8 mice in each group (DEX n=6).

FIG. 7: (A) Shows VEGF levels in the retinal tissue of a mouse model of experimental autoimmune uveitis (EAU), using B10.RIII mice. The groups of mice were treated intravitreally with nomacopan (noma), PAS-nomacopan (PAS-noma), anti-VEGF antibody (anti-VEGF) or saline. The retinal tissue was harvested 21 days after induction. Non-induced, untreated, healthy mice were used as a passive control (“healthy ct”). There were six mice per intravitreal injected group and two non-induced untreated (“healthy ct”). The retinal tissue was analysed for VEGF levels using an ELISA assay. All assays were performed twice on replicate samples. (B) shows the clinical score in EAU mice pre treatment at 15 days post immunization and following intravitreal administration of nomacopan-type proteins on day 18 post immunization. Evaluation was carried out on day 15 and day 26. (C) shows stacked clinical scores.

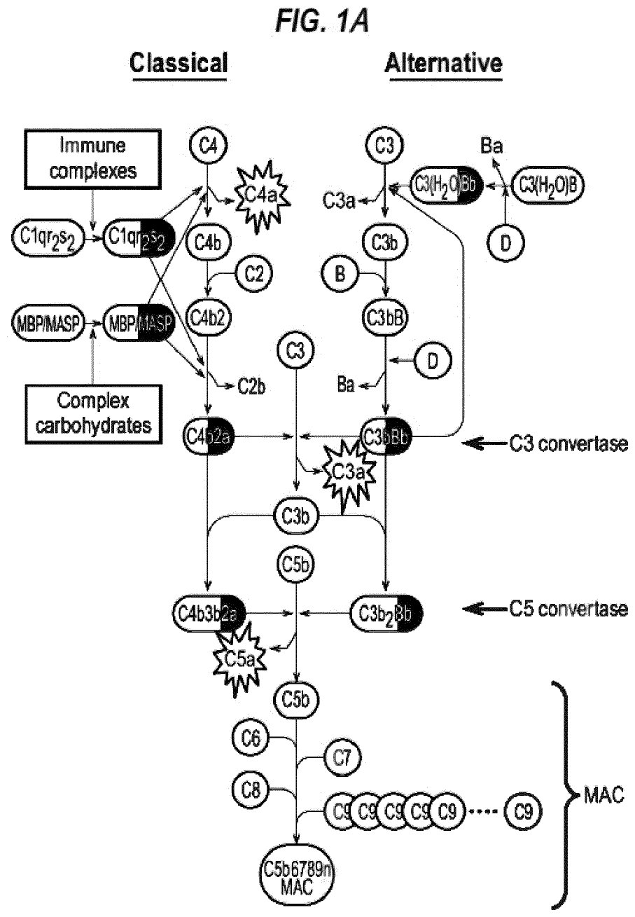

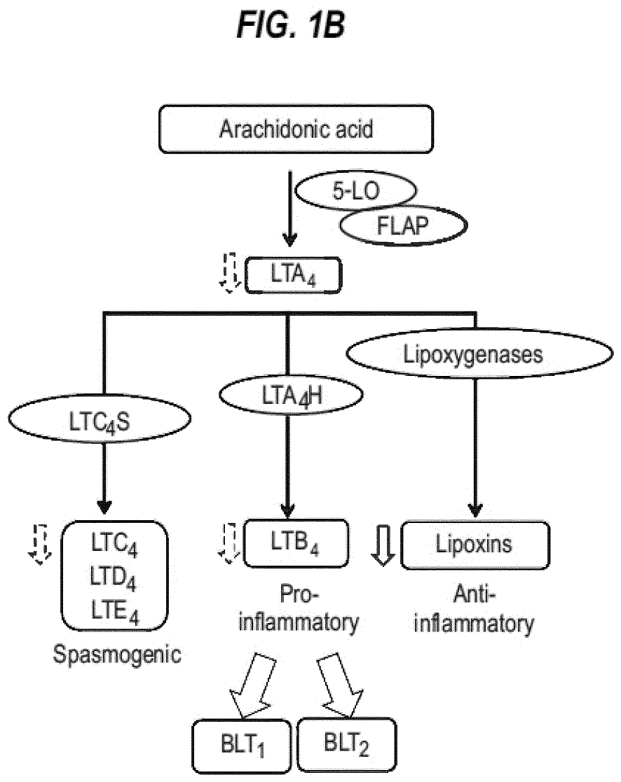

### EXAMPLES

Materials and Methods for Examples 1-3

EAU Induction

EAU was induced as previously described [61], by immunising C57/B16 mice (female, aged 5-7 weeks, Charles River) s.c. with 300 μg of interphotoreceptor retinoid-binding protein (IRBP) 161-180 peptide (SGIPYIISYLHPGNTILHVD, Cambridge Peptides, Cambridge, U.K.) in PBS emulsified with Complete Freud's Adjuvant (CFA, Sigma-Aldrich, Gillingham, U.K.) supplemented with Mycobacterium tuberculosis (Difco, Voigt Global Distribution, Lawrence, Kans.). Mice also received 0.4 μg of pertussis (Sigma-Aldrich) i.p. Treatment

Retinal inflammation was monitored from day 14 onwards to score (mice were only included in the study when retinal inflammation is detected, as assessed by retinal fundoscopy on day 14 onwards), according to previous studies [62]). Mice were harvested on days 1, 2 and 5 (n=6 per group).

For intravitreal administration, mice were treated on days 15 and 18 post immunization with 1-2 μl nomacopan (5 mg/ml), PAS-nomacopan (20 mg/ml), PAS-L-nomacopan (20 mg/ml), dexamethasone or saline. For topical administration the mice were treated twice daily with 2-5 μl of nomacopan (5 mg/ml), PAS-nomacopan (20 mg/ml), L-nomacopan (5 mg/ml), dexamethasone (Maxidex; DEX) or saline for 7 days. Disease progression was graded clinically by retinal fundoscopy pre- and post-treatment and scored histologically.

EAU Scoring

Mice were assessed as previously described regularly for signs of EAU by regular funduscopic observation (e.g. using a Micron III fundus camera (Phoenix Research Labs, Pleasanton, Calif.)). Clinical scoring was based on observations of the retinal optical disk, retinal vessels, retinal tissue infiltrate and structural damage.

One eye from each mouse was harvested at the conclusion of the experiment and fixed and embedded for histological scoring (e.g. enucleated and fixed in 4% glutaraldehyde for 1 h and transferred to 10% formaldehyde for at least 24 h, embedded, processed for H&E staining, and examined histologically). Scores were assigned on a scale of 0-5, according to the criteria for EAU scoring, based on the level of the immune cell infiltration and the degree of retinal damage, as previously described [63]. Immunofluorescence staining for monocyte, neutrophil and T cell infiltration can be performed to identify any change to the infiltrating cell population.

For retinal flow cytometry, retinal layers were collected, minced, filtered and re-suspended in media for ex vivo stimulation (4 hours) and stained for cell surface and intracellular markers 64

### Example 1

### Treatment of EAU With Topical Administration of Nomacopan-Type Proteins

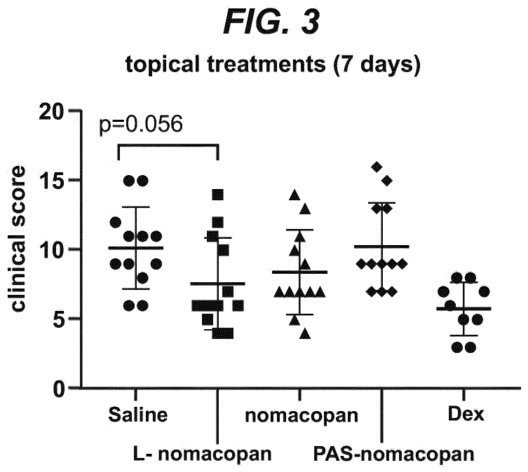

As shown in FIG. 3, following topical administration of nomacopan (5 mg/ml), PAS-nomacopan (20 mg/ml), L-nomacopan (5 mg/ml) the clinical score in EAU mice was lower compared to mice treated only with saline, with the results being statistically significant for L-nomacopan. Trends for nomacopan and PAS-nomacopan treated mice were observed. The clinical score for L-nomacopan treated mice was in line with the clinical score for dexamethasone treated mice. Mean score±SEM; 8.083±0.848; n=12) relative to saline controls (10.33±0.666; n=12; P=0.048), with no significant changes in CD4+ T cell numbers or subtypes in treated mice.

These results are surprising as it was not previously known that the test molecules could pass through the cornea. The reduced efficacy of PAS-nomacopan compared to the un-PASylated version may reflect the fact that the PASylated version is larger.

### Example 2

### Coexpression of BLT1 and C5a Receptors in Mouse Retinal Cells

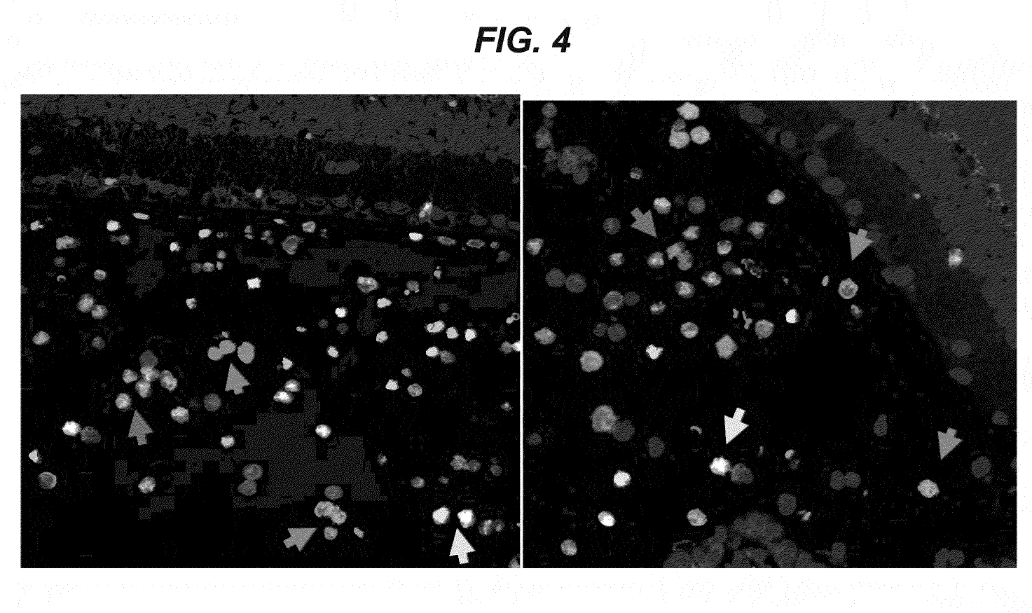

Confocal microscopy of mouse retinal sections in UAE mice using monoclonal antibodies recognising BLT1 and C5a receptors, counterstained with DAPI (blue to indicated nuclei) shows both receptors expressed in inflammatory cells. Some individual cells co-expressed both receptors, whilst many single receptor-expressing cells were also observed. It is believed that at least some of the cells are M2 macrophages which are known to migrate to areas of retinal damage where they release VEGF in response to LTB4 stimulation (see FIG. 4, 7 and Example 4).

This is consistent with other results presented herein that nomacopan-type proteins can be useful agents in treatment of such diseases (e.g. diseases in which there is an LTB4 involvement and/or involvement of the complement pathway).

### Example 3

### Treatment of EAU With Intravitreal Administration of Nomacopan-Type Proteins

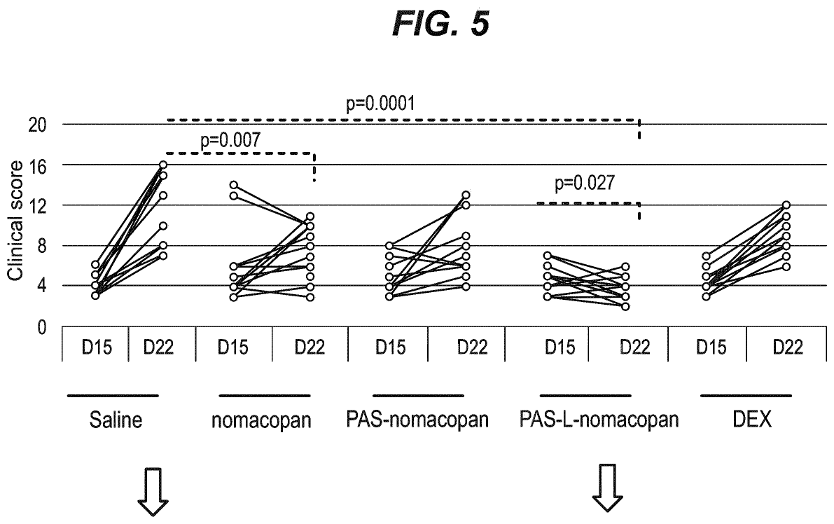

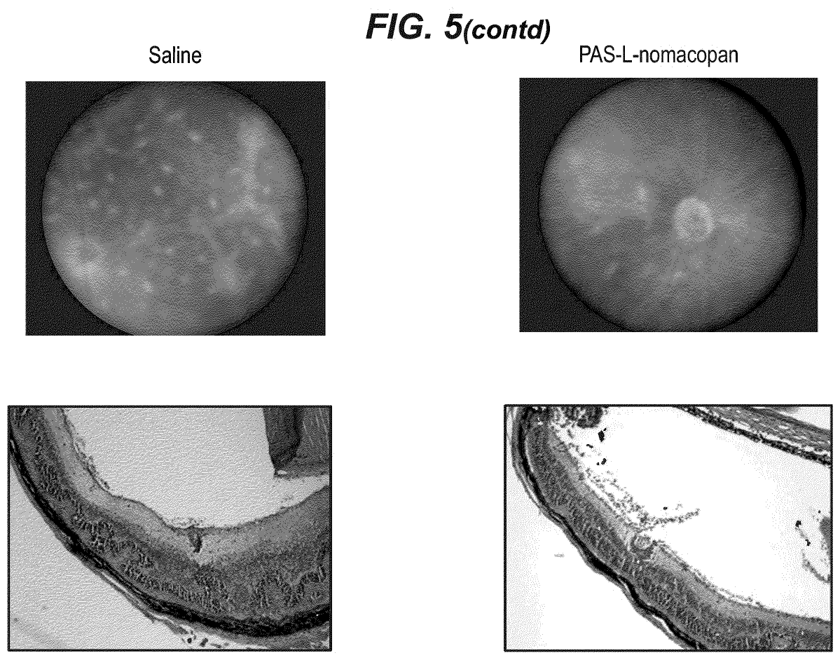

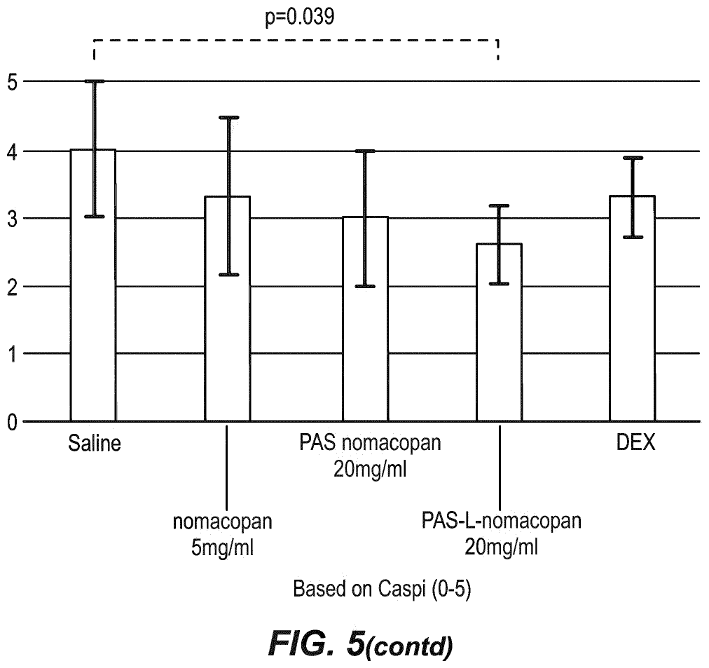

As shown in FIG. 5, following intravitreal administration of nomacopan-type proteins on days 15 and 18 (nomacopan (5 mg/ml), PAS-nomacopan (20 mg/ml), PAS-L-nomacopan (20 mg/ml), each administered in a volume of 1-2 μl to EAU mice, the observed clinical scores at day 22 was in general lower than those in the control (saline treated) mice, and the increase in clinical score between the two time points (day 15 and day 22) was less for the treated mice than for the control (saline treated) mice. PAS-nomacopan mitigated disease progression post intravitreal administration as determined by clinical scoring (mean score±SEM; 3.813±1.014; n=16) relative to saline controls (12.36±1.014; n=16; P=0.0001).

FIG. 5 also shows the composite histological scores for the various treatments. Again there was a lower clinical score following treatment with each of the Nomocopan type molecules, compared to the control (saline treated) mice, and a statistically significant difference between the control (saline treated) mice composite histological scores and the composite histological score for the PAS-L-nomacopan (20 mg/ml) treated mice.

By way of example, and as also shown in FIG. 5, the retinal folding (one of the components of the composite histological score) is visually very different in control (saline treated) mice compared to in the PAS-L-nomacopan (20 mg/ml) treated mice.

These data show for the first time that confirm that nomacopan-type proteins are bioactive when injected intravitreally (i.e. that they have the ability to penetrate Bruch's member and/or the inner limiting membrane to have their effect on cells residing within the choroid). This is to be contrasted with other proteinaceous molecules that have been tested for eye treatments, such as Lampalizumab [65]

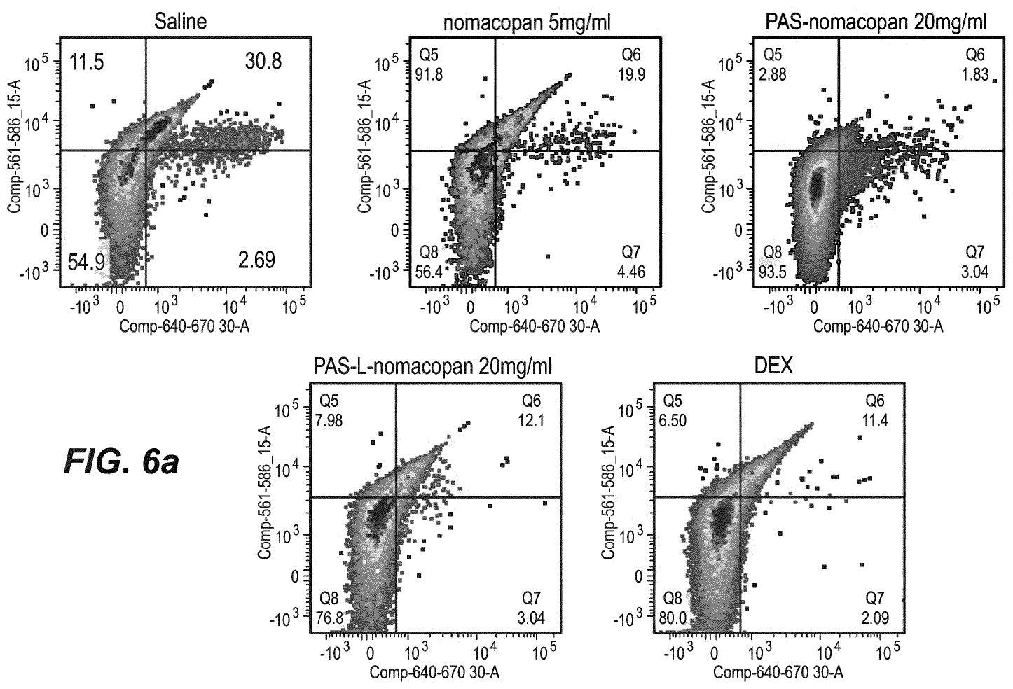

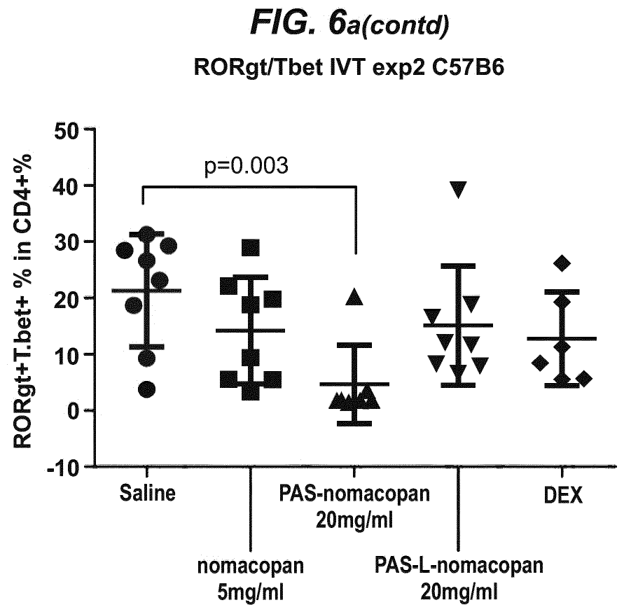

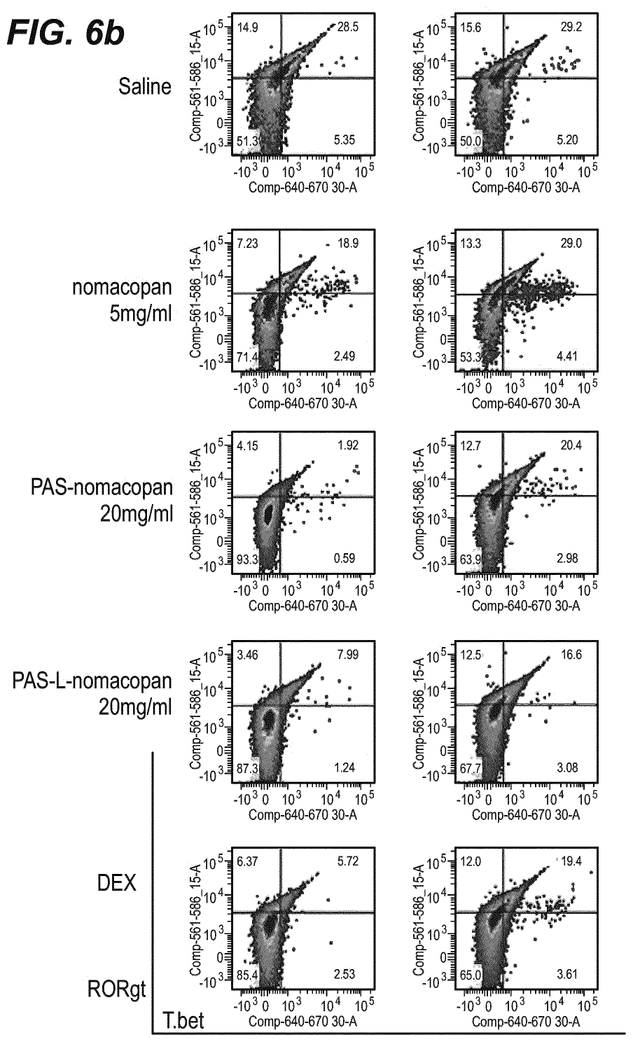

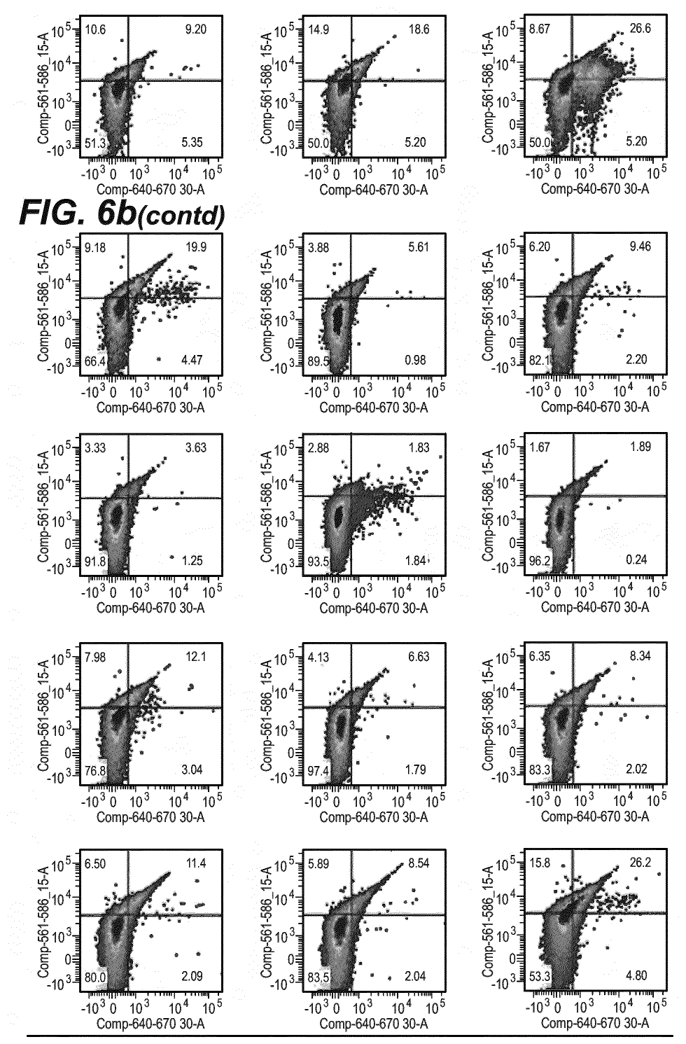

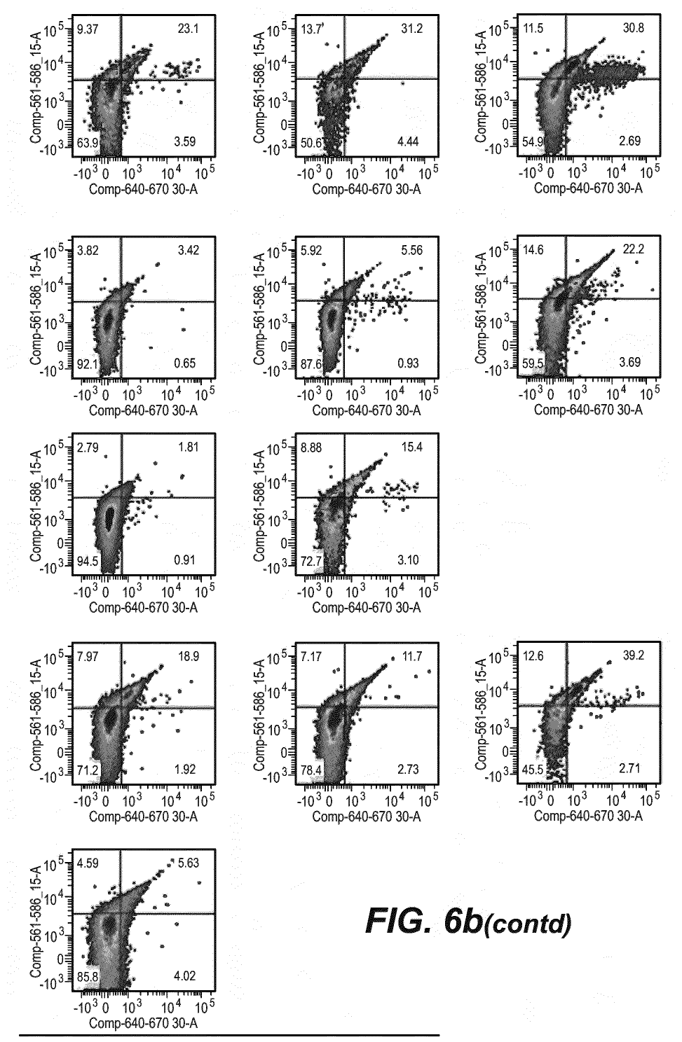

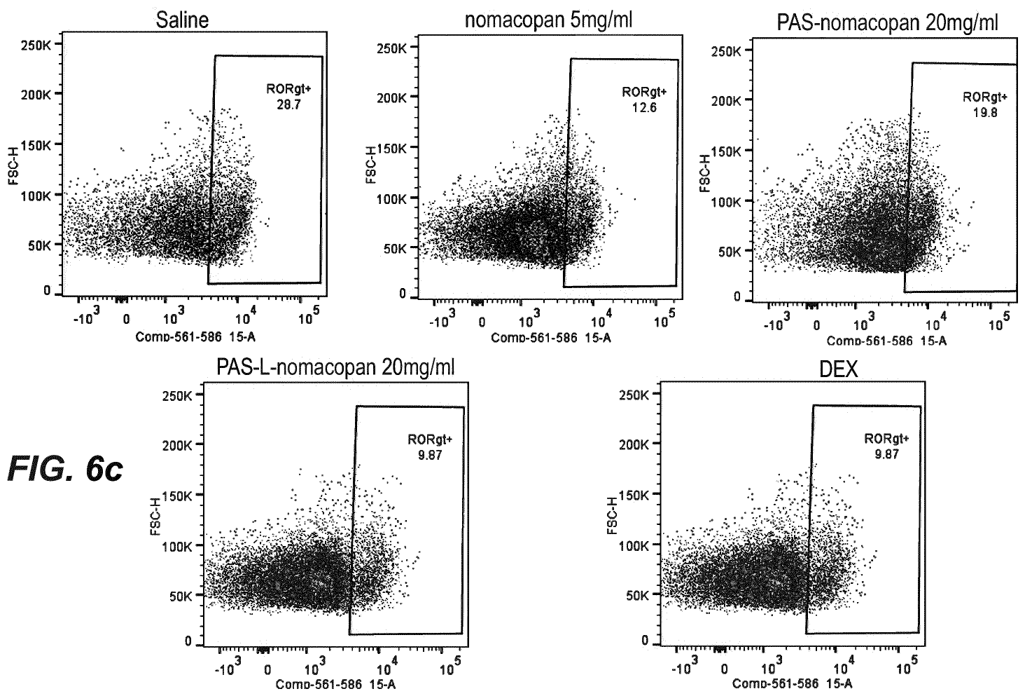

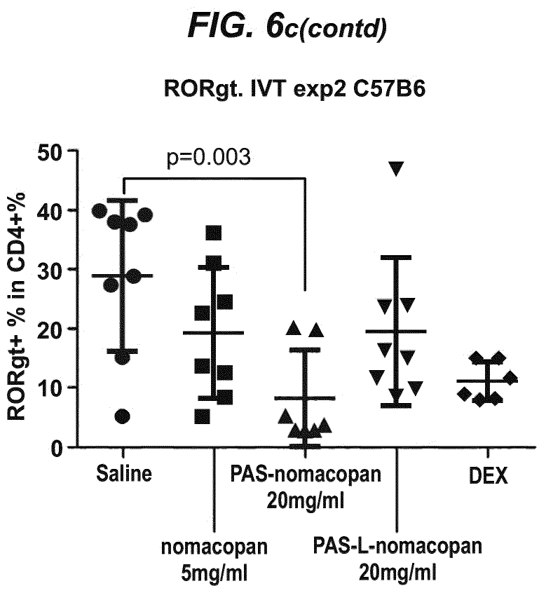

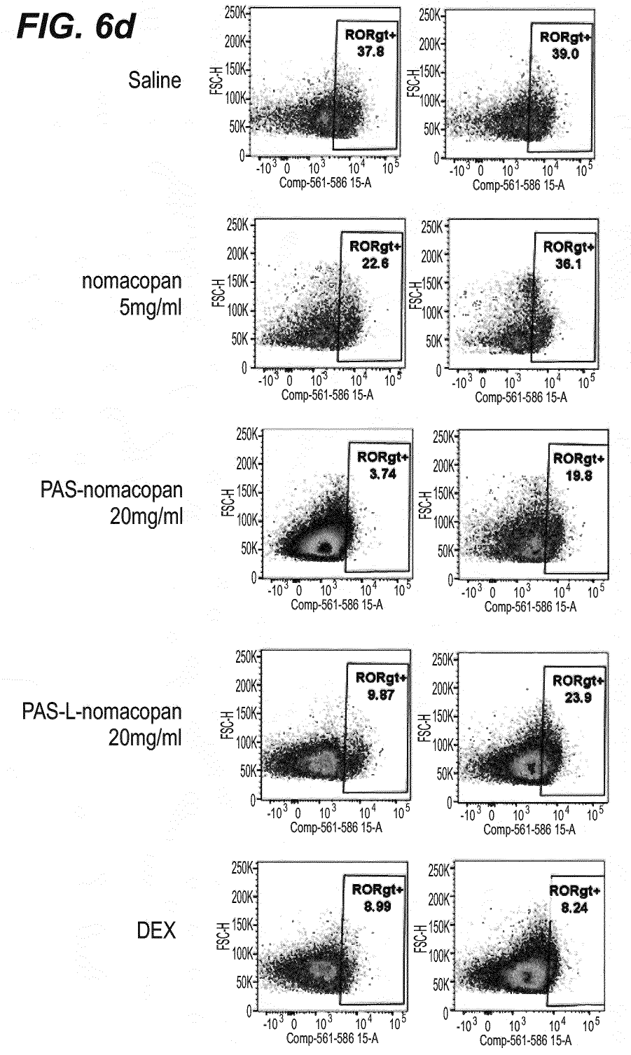

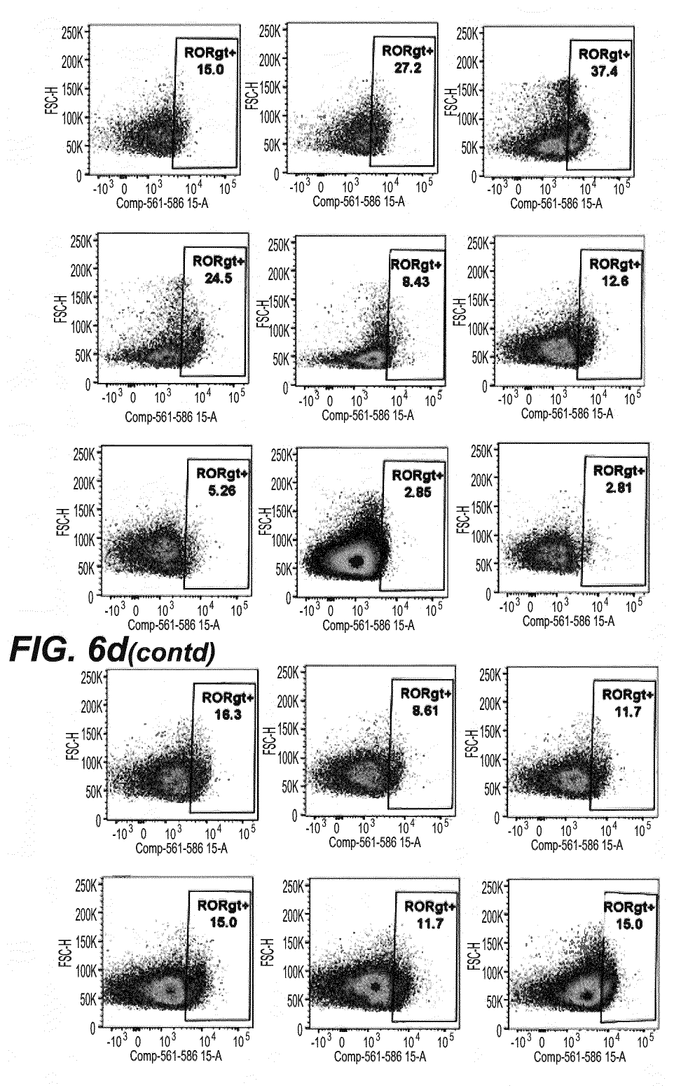

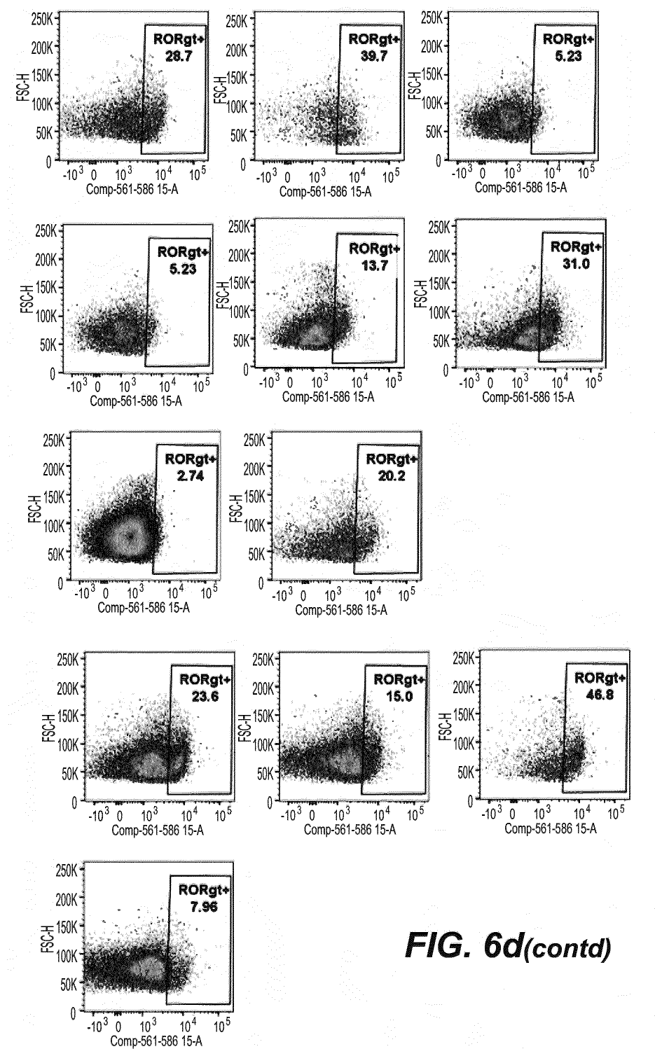

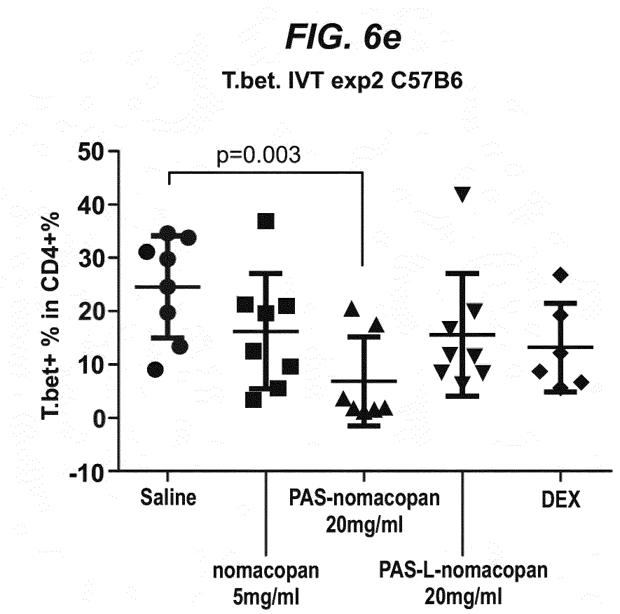

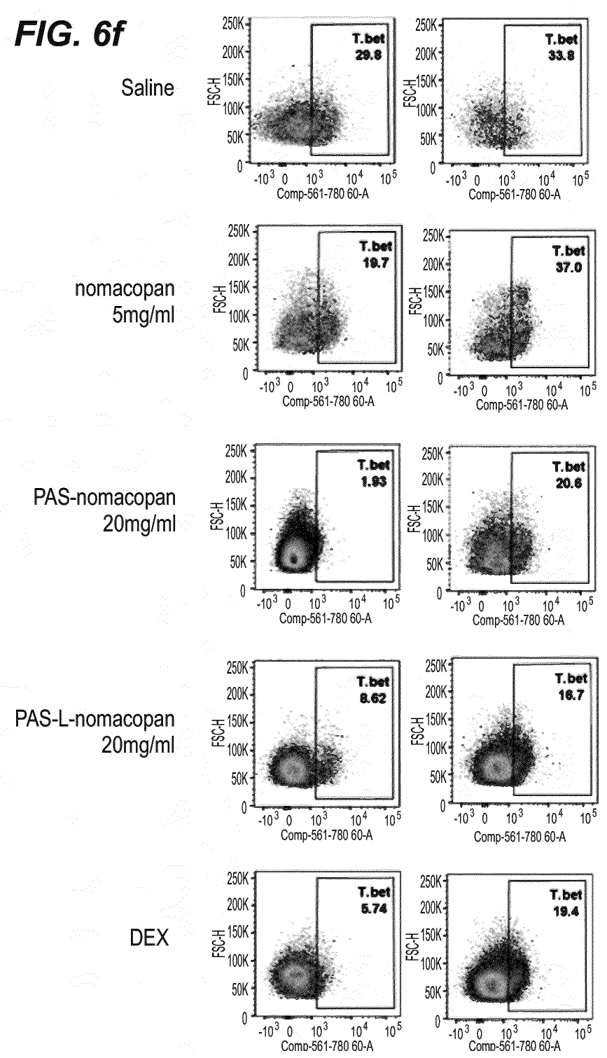

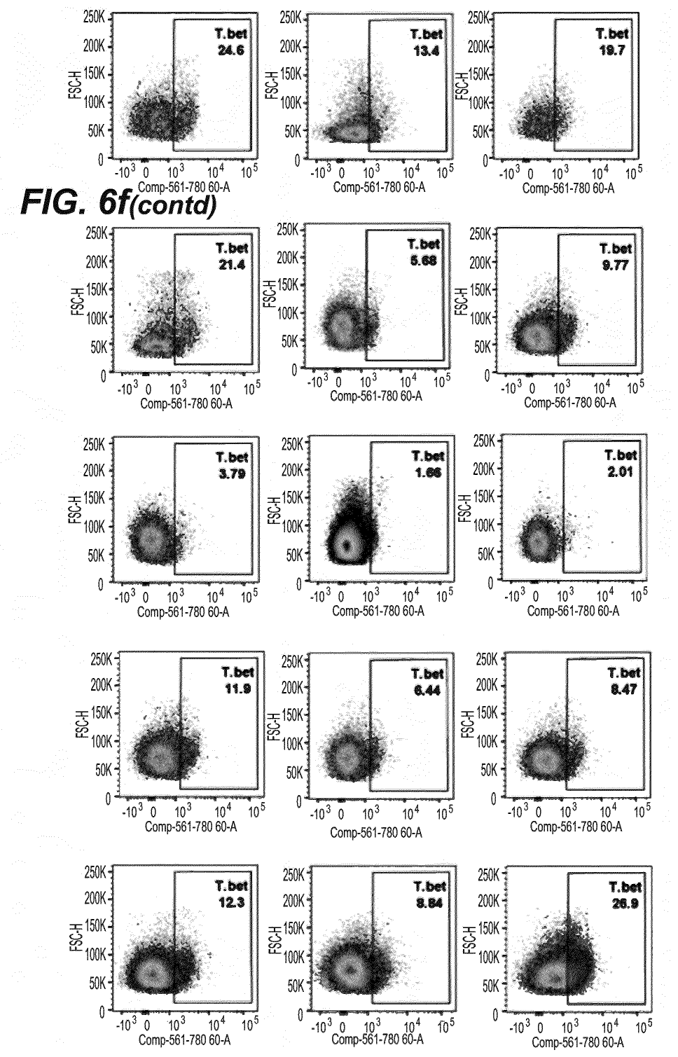

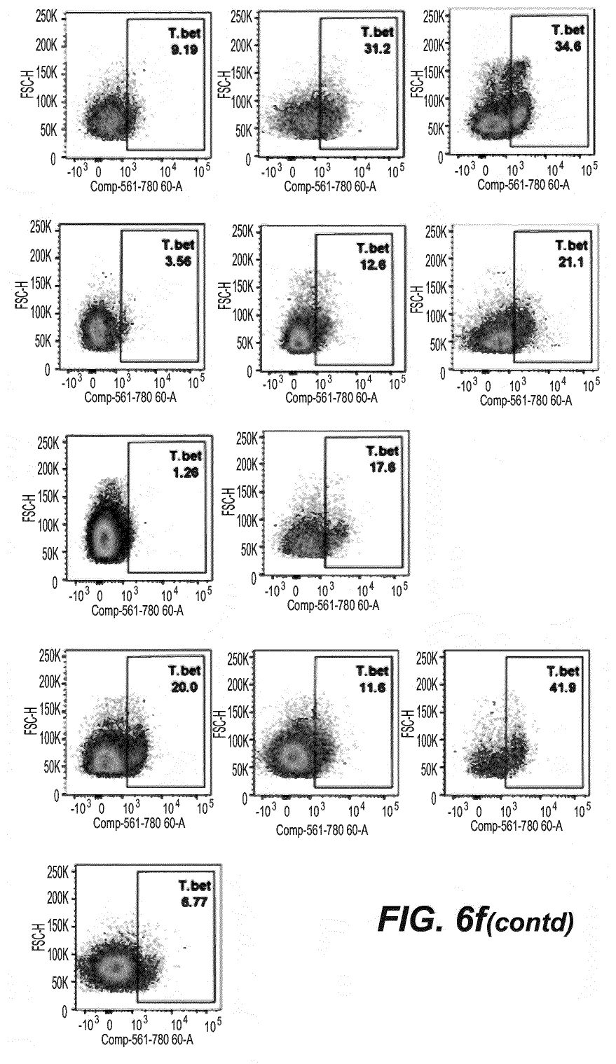

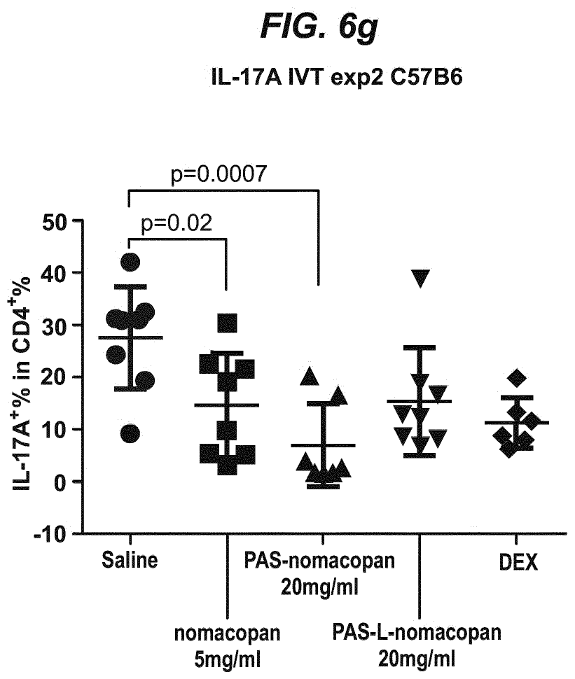

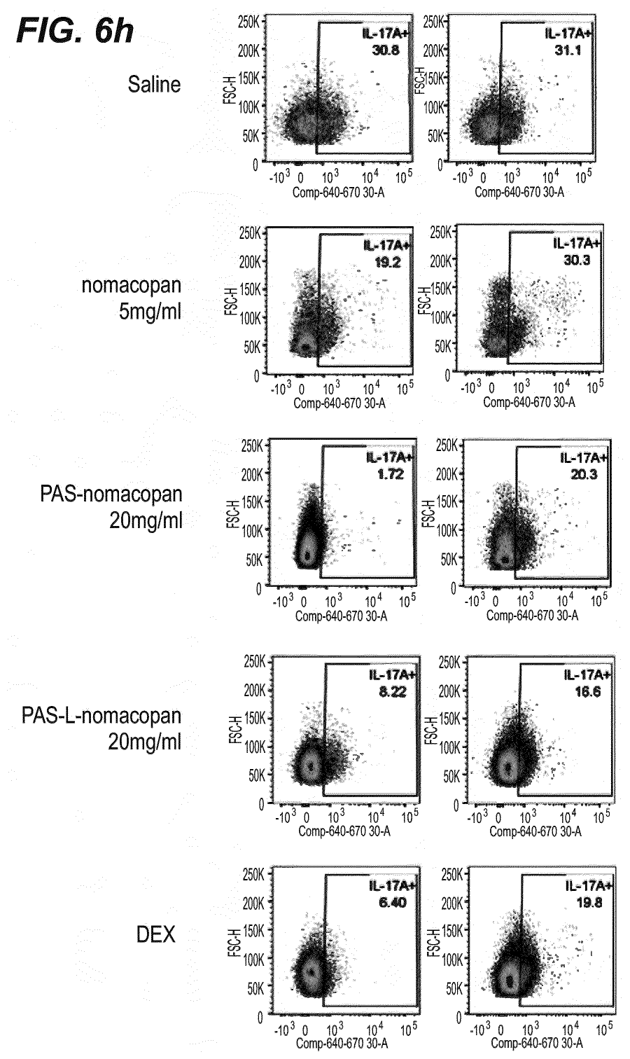

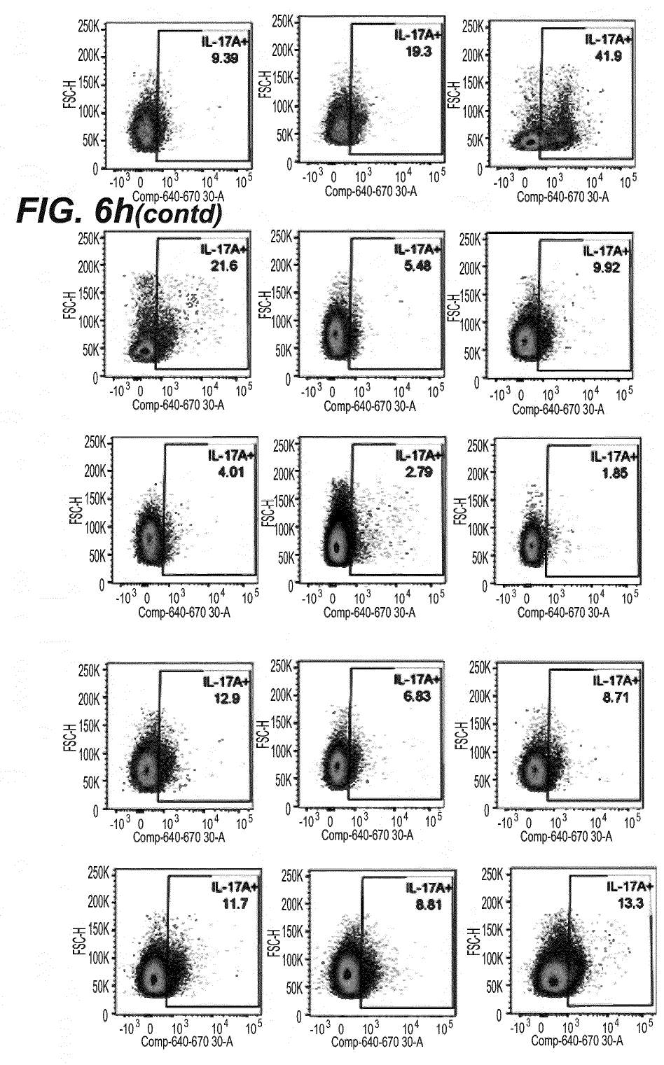

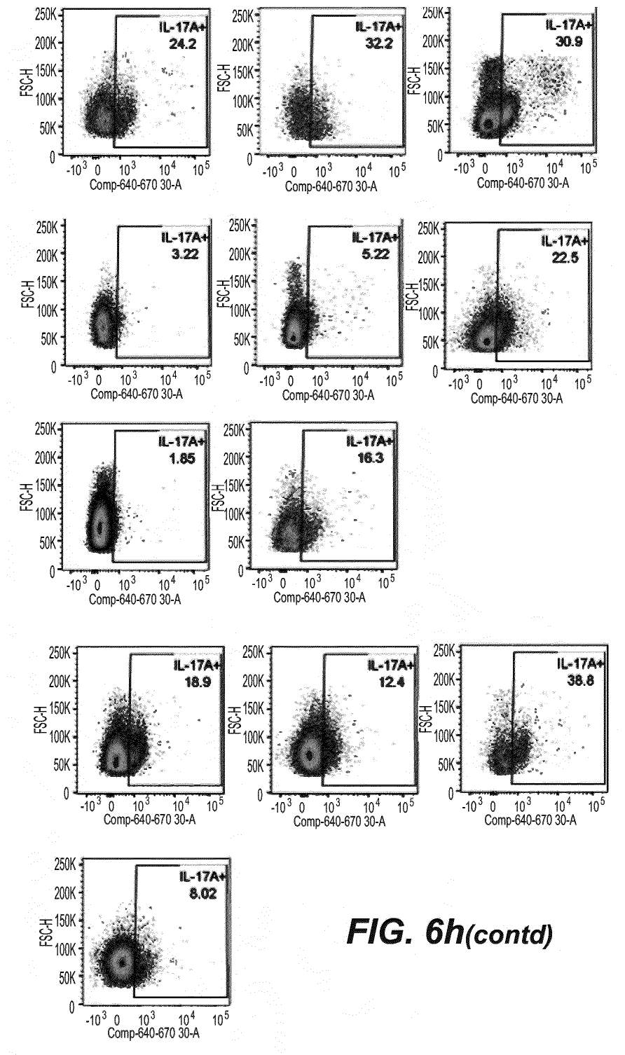

RORgt/T.bet+ expression in CD4+ infiltrating cells in the EAU mice was also analysed by flow cytometry (see FIG. 6a-b which shows the percentage of RORgt/T.bet cells in CD4+ cells, with RORgt expression on the y axis and Tbet expression on the x axis). RORgt and Tbet are Tcell markers that are associated with the Th17 cell type and IL-17 expression. The percentage of RORgt/T.bet+ expressing cells in CD4+ infiltrating cells in the EAU mice was lower in nomacopan type protein treated mice than in control (saline treated) mice. The levels in PAS-nomacopan (20 mg/ml) treated mice were lowest and the difference between the levels in these mice and in the control (saline treated) mice was statistically significant. A significant decrease in retinal double-positive Th1/Th17 (RORγt⁺T-bet⁺) cells (4.717±2.627; n=8) was also observed in PAS-nomacopan-treated EAU mice as compared to saline controls (21.28±3.544; n=8; P=0.003) after twice intravitreal treatments (day 15 and 18) while there was no significant difference in the expression levels of other T cell subsets (data not shown).

RORgt+ expression alone and T.bet expression alone was also analysed in these cells (see FIG. 6c-6f).

IL-17A expression was also analysed in these cells (see FIG. 6g-6h). Decreases in Th17 (IL-17A⁺CD4⁺) cells in the nomacopan and PAS-nomacopan treated groups (mean %±SEM; 14.67±3.534 and 7.019±3.005 respectively, n=8) were detected relative to saline controls (27.47±3.461; n=8; P=0.02 and 0.0007).

### Example 4

### VEGF Levels in EAU Mice and Treatment With Intravitreal Administration of Nomacopan-Type Proteins

Materials and Methods

EAU Induction

EAU was induced by immunising B10.RIII mice (female, aged 5-7 weeks, colony bred in house) s.c. with 300 μg of interphotoreceptor retinoid-binding protein (IRBP) 161-180 peptide (SGIPYIISYLHPGNTILHVD). An appropriate protocol for EAU is set out in [61], in which IRBP (Cambridge Peptides, Cambridge, U.K.) in PBS is emulsified with Complete Freud's Adjuvant (CFA, Sigma-Aldrich, Gillingham, U.K.) supplemented with Mycobacterium tuberculosis (Difco, Voigt Global Distribution, Lawrence, Kans.). In this protocol mice also receive 0.4 μg of pertussis (Sigma-Aldrich) i.p.

Treatment and VEGF Measurement

In an initial experiment, retinal tissue was harvested after induction to determine VEGF levels. The retinal tissue was analysed for VEGF levels using an ELISA assay.

In a subsequent experiment, the retinal tissue was harvested 21 days after induction. Groups of mice were treated intravitreally on days 15 and 18 post induction with 1-2 μl nomacopan (5 mg/ml), L-nomacopan (5 mg/ml) PAS-nomacopan (20 mg/ml), PAS-L-nomacopan (20 mg/ml), anti-VEGF antibody (50 μg/ml) BioLegend (Ultra-Leaf purified anti-mouse VEGF A antibody) or saline. Non-induced, untreated, healthy mice were used as a passive control (“healthy ct”). There were six mice per intravitreal injected group and two non-induced untreated healthy mice (“healthy ct”). The retinal tissue was analysed for VEGF levels using an ELISA assay with mouse VEGF, from R&D (Mouse Quantikine ELISA). All assays were performed twice on replicate samples.

Results

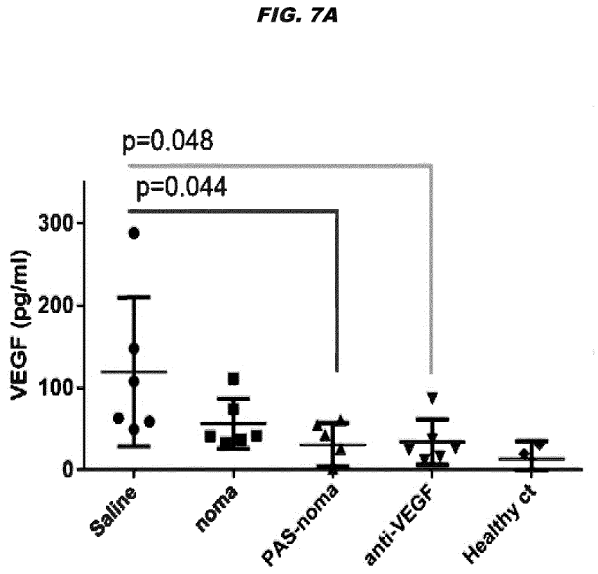

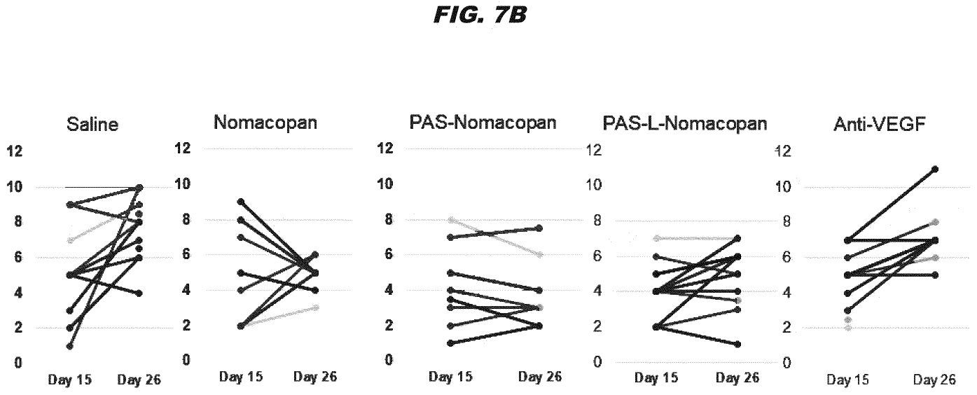

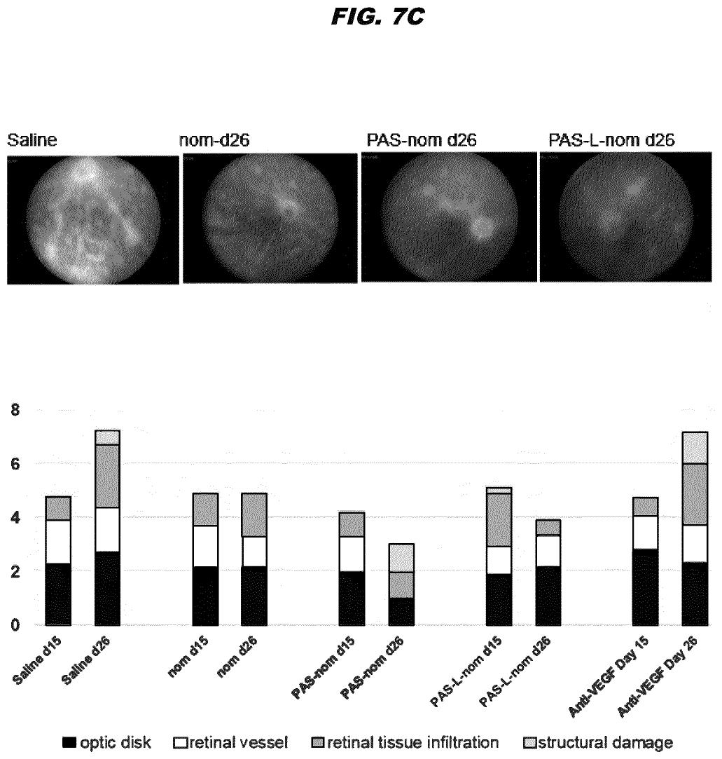

VEGF levels in the retinal tissue of these groups are shown in FIG. 7A. In the initial experiment, induction with IRBP resulted in a significant increase in VEGF levels compared to normal controls (data not shown). An increase can also been seen in FIG. 7A when comparing “saline” with “healthy ct”.

FIG. 7A shows that PAS-nomacopan is at least as effective as the anti-VEGF antibody in reducing retinal VEGF concentration in this EAU model. As nomacopan and its variants are not known to have any direct effect on VEGF, it is thought that the reduction of VEGF levels is an indirect effect of inhibiting LTB4-based activation of M2 macrophages, which is presumed to be the principal source of VEGF in this model [35].

While, L-nomacopan and PAS-L-nomacopan were also tested, the available results for L-nomacopan were inconclusive. The inconclusive results were thought to be the result of an experimental error, so are not shown here.

The scavenging of LTB4 from neutrophils by nomacopan-type proteins is thought to also have the effect of reducing the retinal to peripheral blood LTB4 concentration gradient thereby reducing inflammatory cell trafficking and of inhibiting activation of Th-17 cells (as shown in example 3) and macrophages. The additional reduction of complement C5a and membrane attack complex through the inhibitory effect of nomacopan and PAS-nomacopan on complement C5 may also further reduce the secondary release of VEGF through reducing the C5a concentration gradient and reducing activation of inflammatory cells including Th cells, neutrophils and mononuclear/macrophages. Reducing the trafficking of neutrophils may also reduce the amount of LTB4 available for release.

Clinical scores were taken as shown in FIGS. 7B and 7C.

The anti-VEGF antibody had little or no effect on inflammation in this model whereas suppression of inflammation was observed with certain test molecules.

[1] Hoover et al, 1984, Proc. Nat. Acad. Sci. U.S.A. 81, 2191-2193

[2] Harrison and Murphy, 1995, J. Biol. Chem. 270, 17273-17276

[3] Ford-Hutchinson, 1990, Crit. Rev. Immunol. 10, 1-12

[4] Showell et al., 1995, J. Pharm. Exp. Ther. 273, 176-184

[5] Klaas et al, 2005 J. Exp. Med. 201, 1281-1292

[6] Del Prete et al, 2007 Blood, 109, 626-631

[7] Miyahara et al, 2006 A llergol Int. 55, 91-7

[8] Taube et al, 2006 J. Immunol. 176, 3157-3164

[9] Yamaoka et al, 1989 J. Immunol. 143, 1996-2000

[10] Yokomizo et al, 1997 Nature 387, 620-624

[11] Yokomizo et al, 2000 J. Exp. Med. 192, 421-432

[12] Tager and Luster, 2003 Prostaglandins Leukot. Essent. Fatty Acids 69, 123-134

[13] Yokomizo et al., 2001, J. Biol. Chem. 276, 12454-12459

[14] Kim, N. D. and Luster, A. D. (2007) The Scientific World Journal 7, 1307-1328.

[15] Kim et al., 2006. J. Exp. Med. 203, 829-835

[16] Noiri et al., 2000 Proc Nat Acad Sci USA 97, 823-828

[17] Lundeen et al., 2006. J. Immunol. 177, 3439-3447

[18] Shao et al, 2006. J. Immunol. 176, 6254-6261

[19] Chen et al., 2006. J. Exp. Med. 203, 837-842

[20] Sebaldt et al., 1990 Proc Natl Acad Sd. U.S.A. 8, 6974-6978

[21] Curry et al., 2005 Journal of the American Animal Hospital Association 41 , 298-309

[22] Dube et al., 1998. Zileuton: the first leukotriene inhibitor for use in the management of chronic asthma. In: Drazen J M, Dahlen S, Lee T H, eds. Five-lipoxygenase Products in Asthma. New York, N.Y. Marcel Dekkar, Inc

[23] Sharma and Mohammed, 2006 Immunopharmacology 14, 10-16

[24] WO2004/106369

[25] Jore, M. M. et al, Nature Structural & Molecular Biology 2016 volume 23, pages 378-386

[26] Xu et al European Journal of Pharmacology 2016 787 94

[27] Anaya J M et al. Autoimmunity: From Bench to Bedside, Chapter 37—Autoimmune uveitis, Bogota (Colombia): El Rosario University Press; 2013 Jul. 18.

[28] Guedes et al, Indian J Ophthalmol. 2016; 64(9): 628-634

[29] Gulati et al. Br J Ophthalmol 2011 95: 162-165

[30] Mitchell and Bradley, Health Qual Life Outcomes. 2006; 4: 97.

[31] Delcourt et al. Investigative Ophthalmology & Visual Science 2017, 58, 6399-6407

[32] Clinical Classification of Age-related Macular Degeneration, Ophthalmology 2013; 120:844-851

[33] Xu and Chen Eur J Pharmacol. 2016 15; 787: 94-104.

[34] Ferrara et al. Annu. Rev. Med. 2007. 58:491-504

[35] Sasaki et al. JCI Insight. 2018 Sep. 20; 3(18). pii: 96902.

[36] Cai & McGinnis, J Diabetes Res. 2016; 2016:3789217

[37] Borke et al. Medscape 2017, Retinal Vein Occlusion (RVO)

[38] The Royal College of Ophthalmologists, Clinical Guidelines Retinal Vein Occlusion (RVO) Guidelines 2017

[39] An international classification of retinopathy of prematurity. The Committee for the Classification of Retinopathy of Prematurity. Arch Ophthalmol. 1984; 102(8):1130-4.

[40] Sankar et al. Cochrane Database of Systematic Reviews 2018, Issue 1. Art. No. CD009734.

[41] Lu et. al. Graefes Arch Clin Exp Ophthalmol, 2017, 255:1057-1062

[42] Battaglia et. al. Br J Ophthalmol, 2015, 99:1268-1270

[43] https://eyewiki.aao.org/Polypoidal_Choroidal_Vasculopathy#cite_ref-Yannuzzi_1_1-0

[44] Roversi, P et al Journal of Biological Chemistry 2013, 288(26) 18789-18802

[45] Guo, R. F. and P. A. Ward, Annu Rev Immunol, 2005, 23: p. 821-52

[46] Ricklin D & Lambris J, Nature Biotechnology, 25: 1265-1275 (2007)

[47] Nishimura, Jet al., New Engl J. Med., 30;7: 632-639 (2014)

[48] Breustedt D. A., Schönfeld D. L., Skerra A. (2006) Comparative ligand-binding analysis of ten human lipocalins. Biochim Biophys Acta 1764(2):161-173.

[49] Roversi et al. (2013) J Biol Chem. 288, 18789-18802

[50] Giclas 1994 (Giclas, P. C. (1994). Classical and alternative pathway evaluation (sections 13.1 and 13.2). In Current Protocols in Immunology, Vol. 3

[51] Terpe K, Appl Microbiol Biotechnol, 60: 523-33, 2003

[52] Schlapschy M, et al Protein Eng Des Sel. 2013 August; 26(8):489-501

[53] Kuhn et al Bioconjugate Chem., 2016, 27 (10), pp 2359-2371

[54] Sambrook et al (2000)

[55] Fernandez & Hoeffler (1998)

[56] Ausubel et al. (1991)

[57] Chiang B et al Adv Drug Deliv Rev. 2018 Feb. 15; 126: 58-66.

[58] https://resources.mdsystems.com/pdfs/datasheets/kge006b.pdf

[59] Remington's Pharmaceutical Sciences; Mack Pub. Co., N.J. 1991

[60] Patel et al Ther. Deliv. (2014) 5(3), 337-365

[61] Gardner et al The American Journal of Pathology 2015 Vol 185(5) 1324-1333

[62] Hertweck et al 2016 Cell reports 15 2756

[63] Gegg et al 2005 J Immunol 174 2327-2335

[64] Eskandarpour et al 2017 The Journal of Immunology 198: 1093-1103

[65] Holz et al JAMA Ophthalmology June 2018 Volume 136, Number 6

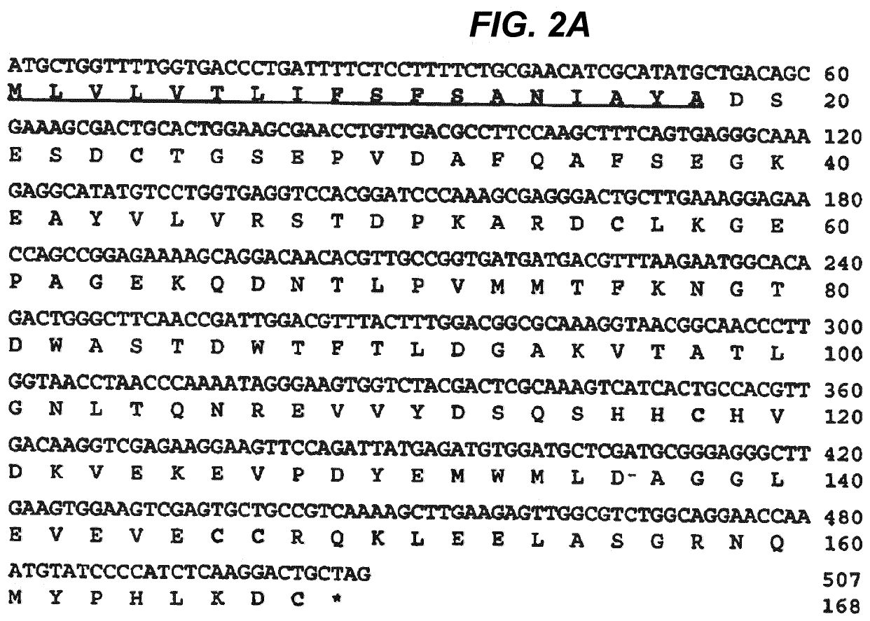

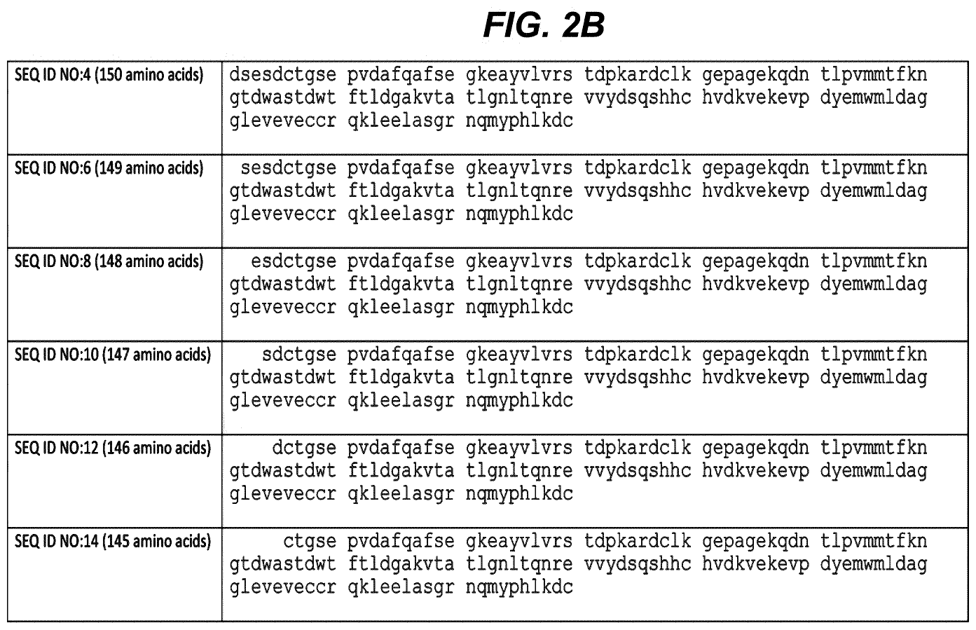

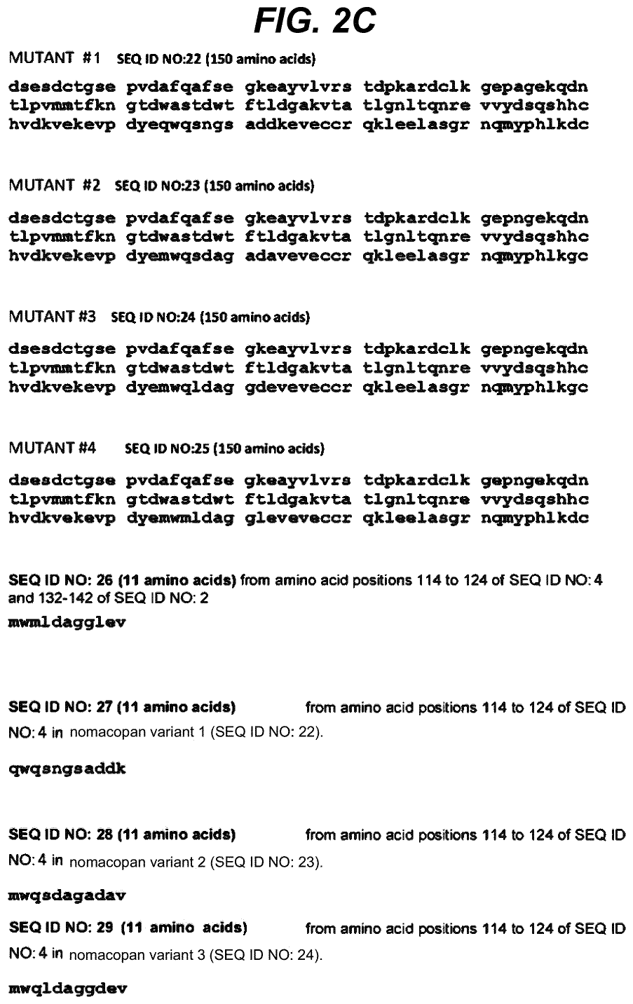

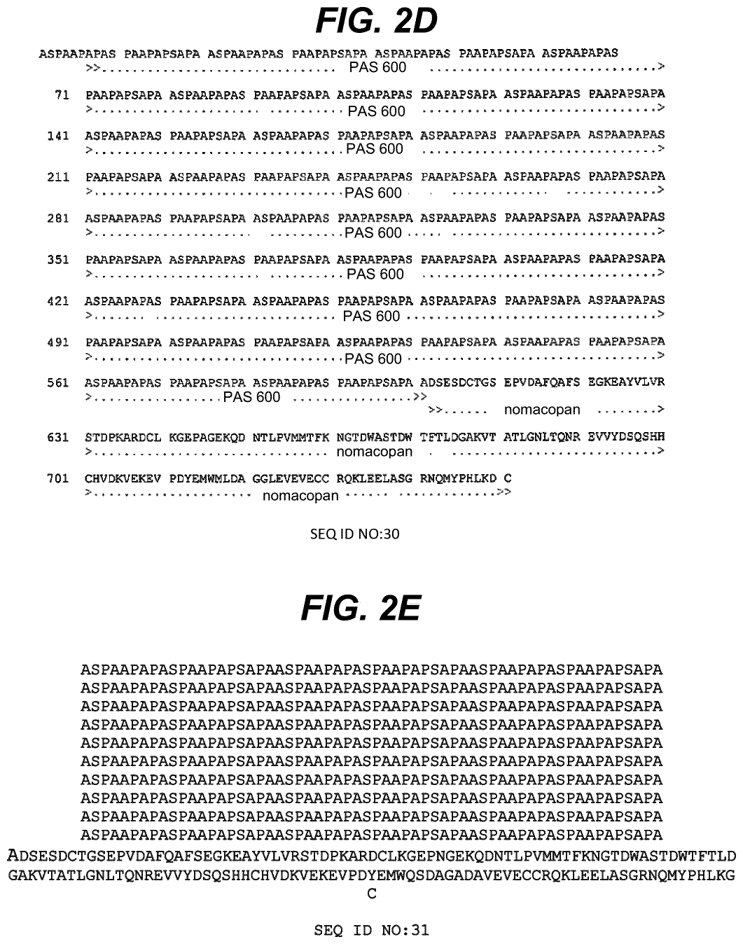

1. A method of treating or preventing a proliferative retinal disease in a subject, which comprises administering to the subject a therapeutically or prophylactically effective amount of an agent which is a protein comprising amino acids 19 to 168 of the amino acid sequence in FIG. 2 (SEQ ID NO: 2) or a functional equivalent of this protein.

2. An agent which is a protein comprising amino acids 19 to 168 of the amino acid sequence in FIG. 2 (SEQ ID NO: 2) or a functional equivalent of this protein for use in a method of treating or preventing a proliferative retinal disease.

3. A method of treating or preventing a proliferative retinal disease in a subject, which comprises administering to the subject a therapeutically or prophylactically effective amount of an, agent which is a nucleic acid molecule encoding a protein comprising amino acids 19 to 168 of the amino acid sequence in FIG. 2 (SEQ ID NO: 2) or a functional equivalent of this protein.

4. An agent which is a nucleic acid molecule encoding a protein comprising amino acids 19 to 168 of the amino acid sequence in FIG. 2 (SEQ ID NO: 2) or a functional equivalent of this protein for use in a method of treating or preventing a proliferative retinal disease.

5. The method of any one of claim 1 or 3, or the agent for use of any one of claim 2 or 4, wherein agent is, or encodes, a protein comprising the sequence of amino acids 19 to 168 of SEQ ID NO: 2, in which up to 50 amino acid substitutions, insertions or deletions have been made,
and the protein binds C5 to prevent the cleavage of complement C5 by convertase into complement C5a and complement C5b and binds to LTB4,
wherein each of the six cysteine amino acids at positions 6, 38, 100, 128, 129, 150 of the mature Nomacopan molecule as set out in SEQ ID NO: 4 is retained and at least five, ten or fifteen or each of the LTB4 binding residues and at least five, ten or fifteen or twenty or each of C5 binding residues set is retained or is subject to a conservative modification, wherein the LTB4 binding residues are Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115 (numbering according to SEQ ID NO:4) and the C5 binding residues are Val26, Val28, Arg29, Ala44, Gly45, Gly61, Thr62, Ser97, His99, His101, Met 114, Met 116, Leu117, Asp118, Ala119, Gly120, Gly121, Leu122, Glu123, Val124, Glu125, Glu127, His146, Leu147 and Asp 149 (numbering according to SEQ ID NO:4).

6. The method or agent for use of claim 5 wherein up to 2, 3, 4, 5, 10, 15, 20 of the LTB4 and C5 binding residues are subject to a conservative modification.

7. The method or agent for use of claim 5 or 6 wherein at least five, ten or fifteen or each of the LTB4 binding residues and at least five, ten or fifteen or twenty or each of the C5 binding residues is retained.

8. The method or agent for use of any of claims 5 to 7, wherein each of the LTB4 binding residues and each of the C5 binding residues is retained or is subject to a conservative modification.

9. The method or agent for use of any of claims 5 to 8, wherein each of the LTB4 binding residues and each of the C5 binding residues is retained or is subject to a conservative modification, wherein up to 2, 3, 4, 5, 10, 15, 20 of the C5 and/or LTB4 binding residues are subject to a conservative modification.

10. The method or agent for use of any of claims 5 to 9, wherein each of the LTB4 binding residues and each of the C5 binding residues is retained.

11. The method of any one of claim 1 or 3 or the agent for use of any one of claim 2, or 4, wherein the agent is, or encodes, a protein comprising a sequence having at least 80% sequence identity to the sequence of amino acids 19 to 168 of SEQ ID NO: 2, and said protein:
(i) binds C5 to prevent the cleavage of complement C5 by convertase into complement C5a and complement C5b and binds to LTB4, or
(ii) binds to LTB4 but has reduced or absent C5-binding activity.

12. The method of any one of claim 1, 3 or 11 or the agent for use of any one of claim 2, 4 or 11, wherein the agent is, or encodes, a protein comprising a sequence having at least 90% sequence identity to the sequence of amino acids 19 to 168 of SEQ ID NO: 2, and said protein:
(i) binds C5 to prevent the cleavage of complement C5 by convertase into complement C5a and complement C5b and binds to LTB4, or
(ii) binds to LTB4 but has reduced or absent C5-binding activity.

13. The method of any one of claims 1, 3, or 11 to 12, or the agent for use of any one of claims 2, 4, or 11 to 12, wherein the agent is, or encodes, a protein comprising a sequence having at least 95% sequence identity to the sequence of amino acids 19 to 168 of SEQ ID NO: 2,
and said protein:
(i) binds C5 to prevent the cleavage of complement C5 by convertase into complement C5a and complement C5b and binds to LTB4, or
(ii) binds to LTB4 but has reduced or absent C5-binding activity.

14. The method of any one of claim 1, 3, or 11, or the agent for use of any one of claim 10 2, 4, or 11, wherein the agent is, or encodes a protein which exhibits LTB4 binding activity and reduced or absent C5 binding, said protein comprising or consisting of SEQ ID NO: 4 in which from 1 to 30 amino acid substitutions are made, wherein
(i) in the positions 114 to 124 of SEQ ID NO: 4 one or more of the following substitutions (a)-(j) is made:
a. Met114 is replaced with Gln, Asp, Asn, Glu, Arg, Lys, Gly, Ala, Pro, His, or Thr;
b. Met116 is replaced with Gln, Asp, Asn, Glu, Arg, Lys, Gly, Ala, Pro, His, or Thr;
c. Leu117 is replaced with Ser, Asp, Asn, Glu, Arg, Lys, Gly, Ala, or Pro;
d. Asp118 is replaced with Asn, Gln, Arg, Lys, Gly, Ala, Leu, Ser, Ile, Phe, Tyr, Met Pro, His, or Thr;
e. Ala119 is replaced with Gly, Asp, Asn, Glu, Arg, Lys, Leu, Ile, Phe, Tyr, Met, Pro, or His;
f. Gly120 is replaced with Ser, Asp, Asn, Glu, Arg, Lys, Leu, Ile, Phe, Tyr, Met, Pro, or His;
g. Gly121 is replaced with Ala, Asp, Asn, Glu, Arg, Lys, Leu, Ile, Phe, Tyr, Met, Pro, or His;
h. Leu122 is replaced with Asp, Glu, Asn, Ala, Gln, Arg, Lys, Pro, or His;
i. Glu123 is replaced with Asp, Ala, Gln, Asn, Arg, Lys, Gly, Leu, Ser, Ile, Phe, Tyr, Pro, His, or Thr;
j. Val124 is replaced with Lys, Gln, Asn, Arg, Lys, Gly, Ala, Pro, His, or Thr; or/and
wherein
(ii) Ala44 in SEQ ID NO: 4 is replaced with Asn, Asp, Gln, Glu, Arg, Lys, Leu, Ile, Phe, Tyr, Met, Pro, or His.

15. The method of any one of claim 1, 3, 11 or 14, or the agent for use of any one of claim 2, 4, 11 or 14, wherein the agent is, or encodes a protein as defined in claim 14, wherein:
a. Met116 is replaced with Gln, Asp, Asn, Glu, Arg, Lys, Gly, Ala, Pro, His, or Thr, preferably Gln;
b. Leu117 is replaced with Ser, Asp, Asn, Glu, Arg, Lys, Gly, Ala, or Pro, preferably Ser;
c. Gly121 is replaced with Ala, Asp, Asn, Glu, Arg, Lys, Leu, Ile, Phe, Tyr, Met, Pro, or His, preferably Ala;
d. Leu122 is replaced with Asp, Glu, Asn, Ala, Gln, Arg, Lys, Pro, or His, preferably Asp; and
e. Glu123 is replaced with Asp, Ala, Gln, Asn, Arg, Lys, Gly, Leu, Ser, Ile, Phe, Tyr, Pro, His, or Thr, preferably Ala or Asp.

16. The method of any one of claims 1, 3, 11 or 14 to 15, or the agent for use of any one of claim 2, 4, 11 or 14 to15, wherein the agent is, or encodes a protein as defined in any of claim 14 or 15, wherein:
in positions 114 to 124 of SEQ ID NO: 4:
f. Met116 is replaced with Gln;
g. Leu117 is replaced with Ser;
h. Gly121 is replaced with Ala;
i. Leu122 is replaced with Asp; and
j. Glu123 is replaced with Ala.

17. The method of any one of claim 1, 3 or 5, 11 or 14 to 16, or the agent for use of any one of claim 2, 4 or 5, 11 or 14 to16, wherein the agent is, or encodes a protein as defined in any one of claims of claims 14 to 16, wherein Trp 115 is not substituted.

18. The method of any one of claims 1, 3 or 5, 11 or 14 to 17, or the agent for use of any one of claim 2, 4 or 5, 11 or 14 to17, wherein the agent is, or encodes a protein as defined in any one of claims 14 to 17, wherein Met114, Trp 115, Asp118, Ala119, Gly120 and Val124 are not substituted.

19. The method of any one of claims 1, 3 or 5, 11 or 14 to 18, or the agent for use of any one of claims 2, 4 or 5 or 14 to 18, wherein the agent is, or encodes a protein as defined in any one of claims 14 to 18, which has a loop sequence between amino acid positions 114 to 124 of SEQ ID NO:4 as set out in SEQ ID NO:28 and which has 1-20 additional substitutions compared to SEQ ID NO:4 beyond those that are set out in SEQ ID NO:23.

20. The method of any one of claims 1, 3 or 5, 11 or 14 to 19, or the agent for use of any one of claims 2, 4 or 5, 11 or 14 to 19, wherein the agent is, or encodes a protein as defined in any one of claims 14 to 19, which has 2-15 additional substitutions compared to SEQ ID NO:4 beyond those that are set out in SEQ ID NO:23.

21. The method of any one of claims 1, 3 or 5, 11 or 14 to 20, or the agent for use of any one of claims 2, 4 or 5, 11 or 14 to 20, wherein the agent is, or encodes a protein as defined in any of claims 14 to 20, which has 3-10 additional substitutions compared to SEQ ID NO:4 beyond those that are set out in SEQ ID NO:23.

22. The method of any one of claim 1, 3 or 5, 11 or 14 to21, or the agent for use of any one of claim 2, 4 or 5, 11 or 14 to21, wherein the agent is, or encodes a protein as defined in any of claims 14 to 21, wherein Ala44 in SEQ ID NO: 4 is replaced with Asn, Asp, Gln, Glu, Arg, Lys, Leu, Ile, Phe, Tyr, Met, Pro, or His.

23. The method of any one of claim 1, 3 or 5, 11 or 14 to22, or the agent for use of any one of claim 2, 4 or 5, 11 or 14 to22, wherein the agent is, or encodes a protein as defined in any of claims 14 to 22, wherein Ala44 in SEQ ID NO: 4 is replaced with Asn.

24. The method of any one of claim 1, 3 or 5, 11 or 14 to23, or the agent for use of any one of claim 2, 4 or 5, 11 or 14 to23, wherein the agent is, or encodes a protein as defined in any of claims 14 to 23, wherein Asp149 in SEQ ID NO: 4 is replaced with Gly, Gln, Asn, Ala, Met, Arg, Lys, Leu, Ser, Ile, Phe, Tyr, Pro, His, or Thr.

25. The method of any one of claim 1, 3 or 5, 11 or 14 to24, or the agent for use of any one of claims 2, 4 or 5, 11 or 14 to 24, wherein the agent is, or encodes a protein as defined in any of claims 14 to 24, wherein Ala44 in SEQ ID NO: 4 is replaced with Asn and Asp149 in SEQ ID NO: 4 is replaced with Gly.

26. The method of any one of claims 1, 3 or 5, 11 or 14 to 25, or the agent for use of any one of claims 2, 4 or 5, 11 or 14 to 25, wherein the agent is, or encodes a protein as defined in any one of claims 14 to 25, wherein the six cysteine amino acids at positions 6, 38, 100, 128, 129, 150 of SEQ ID NO: 4 are retained in unmodified form.

27. The method of any one of claims 1, 3 or 5, 11 or 14 to 26, or the agent for use of any one of claims 2, 4 or 5, 11 or 14 to 26, wherein the agent is, or encodes a protein as defined in any one of claims 14 to 25, wherein one or more of the following amino acids is not substituted: Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115.

28. The method of any one of claims 1, 3 or 5, 11 or 14 to 27, or the agent for use of any one of claims 2, 4 or 5, 11 or 14 to 27, wherein the agent is, or encodes a protein as defined in any one of claims 14 to 27, wherein all of the following amino acids are not substituted: Phe18, Tyr25, Arg36, Leu39, Gly41, Pro43, Leu52, Val54, Met56, Phe58, Thr67, Trp69, Phe71, Gln87, Arg89, His99, His101, Asp103, and Trp115.

29. The method of any one of claims 1, 3 or 5, 11 or 14 to 27, or the agent for use of any one of claims 2, 4 or 5, 11 or 14 to 27, wherein the agent is, or encodes a protein as defined in claims 14 to 27 wherein:
a. none of amino acids 5, 6, 11, 13-15, 20-21, 24-27, 29-32, 35-41, 45, 47-48, 50, 52-60, 64, 66, 69-81, 83, 84, 86, 90-94, 97-104, 112-113, 115, 125-129, 132-139, 145, 148, and 150 in SEQ ID NO:4 are substituted; or
b. none of amino acids 5, 6, 11, 13-15, 18, 20-21, 24-27, 29-32, 35-41, 43, 45, 47-48, 50, 52-60, 64, 66, 67, 69-81, 83, 84, 86, 87, 89, 90-94, 97-104, 112-113, 115, 125-129, 132-139, 145, 148, and 150 in SEQ ID NO:4 are substituted; or
c. none of amino acids 5, 6, 11, 13-15, 18, 20-21, 24-25, 27, 30-32, 35-41, 43, 47-48, 50, 52-60, 64, 66, 67, 69-81, 83, 84, 86, 87, 89, 90-94, 98, 100, 102-104, 112-113, 115, 126, 128-129, 132-139, 145, 148, and 150 in SEQ ID NO:4 are substituted.

30. The method of any one of claims 1, 3 or 5 to 13, or the agent for use of any one of claims 2, 4 or 5 to 13, wherein the agent is, or encodes a protein comprising or consisting of (i) the sequence of amino acids 19 to 168 of SEQ ID NO: 2, (ii) SEQ ID NO:22, (iii) SEQ ID NO:23, (iv) SEQ ID NO:24 or (v) SEQ ID NO:25,
preferably wherein the agent is, or encodes a protein comprising or consisting of the sequence of amino acids 19 to 168 of SEQ ID NO: 2, or SEQ ID NO:23.

31. The method of any one of claim 1, 3, 5 to 13, or 30, or the agent for use of any one of claim 2, 4, 5 to 13 or 30, wherein the agent is, or encodes a protein comprising or consisting of the sequence of amino acids 19 to 168 of SEQ ID NO: 2.

32. The method of any one of claim 1, 3 or 14 or 30, or the agent for use of any one of claim 2, 4 or 14, wherein the agent is, or encodes a protein comprising or consisting of SEQ ID NO:22 (variant 1).

33. The method of any one of claims 1, 3 or 14 to 30, or the agent for use of any one of claims 2, 4 or 14 to 30, wherein the agent is, or encodes a protein comprising or consisting of SEQ ID NO:23 (variant 2).

34. The method of any one of claim 1, 3 or 14 or 30, or the agent for use of any one of claim 2, 4 or 14 or 30, wherein the agent is, or encodes a protein comprising or consisting of SEQ ID NO:24 (variant 3).

35. The method of any one of claim 1, 3 or 14 or 30, or the agent for use of any one of claim 2, 4 or 14 or 30, wherein the agent is, or encodes a protein comprising or consisting of SEQ ID NO:25 (variant 4).

36. The method of any preceding claim, or the agent for use of any one preceding claim, wherein the agent is, or encodes, a fragment of the protein as defined in any of claims 1, 2 or 5 to 35, and the protein
(i) binds C5 to prevent the cleavage of complement C5 by convertase into complement C5a and complement C5b and binds to LTB4, or
(ii) binds to LTB4 but has reduced or absent C5-binding activity.

37. The method or the agent for use of any preceding claim wherein the functional equivalent of the protein comprising amino acids 19 to 168 of SEQ ID NO:2 is a fusion protein comprising (a) a sequence as defined in any of claims 5 to 35, or consisting of a fragment as defined in claim 36, and (b) a second sequence, and said fusion protein
(i) binds C5 to prevent the cleavage of complement C5 by convertase into complement C5a and complement C5b and binds to LTB4, or
(ii) binds to LTB4 but has reduced or absent C5-binding activity.

38. The method or agent for use of claim 37 wherein said second sequence is a PAS sequence.

39. The method or agent for use of claim 37 or 38, wherein said fusion protein comprises multiple copies of one of ASPAAPAPASPAAPAPSAPA (SEQ ID NO: 15); AAPASPAPAAPSAPAPAAPS (SEQ ID NO: 16); APSSPSPSAPSSPSPASPSS (SEQ ID NO: 17), SAPSSPSPSAPSSPSPASPS (SEQ ID NO: 18), SSPSAPSPSSPASPSPSSPA (SEQ ID NO:19), AASPAAPSAPPAAASPAAPSAPPA (SEQ ID NO: 20) and ASAAAPAAASAAASAPSAAA (SEQ ID NO: 21), preferably 20-30 or 30 copies of one of SEQ ID NOs 15-21.

40. The method or agent for use of any of claims 37 to 39, wherein said fusion protein comprises (a) a protein consisting of (i) amino acids 19 to 168 of the amino acid sequence in FIG. 2 (SEQ ID NO:2), or (ii) SEQ ID NO:22, 23, 24 or 25,
preferably amino acids 19-168 of SEQ ID NO:2 or SEQ ID NO:22.

41. The method or agent for use of claim 40, wherein said fusion protein comprises a PAS sequence consisting of 30 copies of SEQ ID NO:15.

42. The method or agent for use of any of claims 37 to 41, wherein said fusion protein comprises or consists of SEQ ID NO:30.

43. The method or agent for use of any of claims 37 to 41, wherein said fusion protein comprises or consists of SEQ ID NO:31.

44. The method or the agent for use of any preceding claim, wherein the agent is administered topically to the eye or directly into the eye.

45. The method or the agent for use of any preceding claim, wherein the agent is administered topically to the eye.

46. The method or the agent for use of any preceding claim, wherein the agent is administered directly into the eye, e.g. intravitreally, or suprachoroidally.

47. The method or the agent for use of any preceding claim, wherein the subject is a human.

48. The method or the agent for use of any preceding claim, wherein the proliferative retinal disease is selected from autoimmune uveitis, infective uveitis, wet age-related macular degeneration (choroidal neovascularisation), dry age-related macular degeneration (geographic atrophy), diabetic retinopathy, diabetic macular oedema, optic neuritis (e.g. glaucoma associated optic neuritis), retinal vein occlusion and retinopathy of prematurity, preferably autoimmune uveitis.

49. The method or the agent for use of any preceding claim, wherein the method further comprises the administration of a second proliferative retinal disease treatment.

50. The method or the agent for use of claim 49, wherein the second proliferative retinal disease treatment is selected from an anti-inflammatory medication, an immunomodulatory therapy (IMT) drug, a biologic response modifier (BRM) drug, an anti-VEGF treatment and a second agent which is a protein comprising amino acids 19 to 168 of the amino acid sequence in FIG. 2 (SEQ ID NO: 2) or a functional equivalent of this protein.

51. The method or the agent for use of claim 50, wherein:
(a) the anti-inflammatory medication is a steroid such as a corticosteroid,
(b) the IMT drug is selected from methotrexate, azathioprine, or mycophenolate,
(c) the BRM drug is an anti TNF agent, for example an anti-TNFalpha antibody or fragment thereof such as infliximab or adalimumab,
(d) the anti-VEGF treatment is an anti-VEGF-A antibody or a fragment thereof, such as Bevacizumab (Avastin) or ranibizumab (Lucentis), an anti-VEGF aptamer such as pegaptanib (Macugen) or another anti-VEGF antagonist such as aflibercept (Eylea),
(e) the second agent which is a protein comprising amino acids 19 to 168 of the amino acid sequence in FIG. 2 (SEQ ID NO: 2) or a functional equivalent of this protein is as defined in any one of claims 1 to 43.

52. The method or the agent for use of any of claims 49 to 51, wherein the method comprises administering an agent as defined in any of claims 5 to 36 to the eye topically and administering the second proliferative retinal disease treatment directly into the eye, for example intravitreally or suprachoroidally.

53. The method or the agent for use of claim 52, wherein second proliferative retinal disease treatment is an agent which is a fusion protein, as defined in any of claims 37 to 43.

54. The method or the agent for use of claim 52 or 53, wherein the method comprises administering an agent as defined in any of claim 5 to 31 or 36 to the eye topically and administering a second proliferative retinal disease treatment which is an agent which is a fusion protein, as defined in any of claims 37 to 42 directly into the eye, wherein said agent and said fusion protein each bind C5 to prevent the cleavage of complement C5 by convertase into complement C5a and complement C5b and bind to LTB4.

55. The method or the agent for use of claim 54, wherein the method comprises adminstering a protein comprising or consisting of amino acids 19 to 168 of the amino acid sequence in FIG. 2 (SEQ ID NO: 2) topically to the eye and adminstering the fusion protein of claim 42 directly into the eye.

56. The method or the agent for use of any of claim 52 or 53, wherein the method comprises administering an agent as defined in any of claims 5 to 30 or 32 to 36 to the eye topically and administering a second proliferative retinal disease treatment which is an agent which is a fusion protein, as defined in any of claim 37 to 41 or 43 directly into the eye, wherein said said agent and said fusion protein each bind to LTB4 but have reduced or absent C5 binding activity.

57. The method or the agent for use of claim 56, wherein the method comprises adminstering a protein as defined in claim 32 topically to the eye and adminstering the fusion protein of claim 43 directly into the eye.

58. The method or the agent for use of claim 52, wherein the method comprises administering an agent as defined in any of claims 5 to 30 or 32 to 36 to the eye topically and administering a second proliferative retinal disease treatment which is an anti-VEGF treatment directly into the eye.

59. The method or the agent for use of claims 58, wherein said anti-VEGF treatment is an anti-VEGF-A antibody or a fragment thereof, such as Bevacizumab (Avastin) or ranibizumab (Lucentis), an anti-VEGF aptamer such as pegaptanib (Macugen) or another anti-VEGF antagonist such as aflibercept (Eylea).


SEQUENCE LISTING (32 sequences)

SEQ ID NO: 1 - DNA, 507 bp, Ornithodoros moubata
atgctggttttggtgaccctgattttctccttttctgcgaacatcgcatatgctgacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 2 - protein, 168 aa, Ornithodoros moubata
MLVLVTLIFSFSANIAYADSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 3 - DNA, 453 bp, Ornithodoros moubata
gacagcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 4 - protein, 150 aa, Ornithodoros moubata
DSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 5 - DNA, 450 bp, Ornithodoros moubata
agcgaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 6 - protein, 149 aa, Ornithodoros moubata
SESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 7 - DNA, 447 bp, Ornithodoros moubata
gaaagcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 8 - protein, 148 aa, Ornithodoros moubata
ESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 9 - DNA, 444 bp, Ornithodoros moubata
agcgactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 10 - protein, 147 aa, Ornithodoros moubata
SDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 11 - DNA, 441 bp, Ornithodoros moubata
gactgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 12 - protein, 146 aa, Ornithodoros moubata
DCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 13 - DNA, 438 bp, Ornithodoros moubata
tgcactggaagcgaacctgttgacgccttccaagctttcagtgagggcaaagaggcatatgtcctggtgaggtccacggatcccaaagcgagggactgcttgaaaggagaaccagccggagaaaagcaggacaacacgttgccggtgatgatgacgtttaagaatggcacagactgggcttcaaccgattggacgtttactttggacggcgcaaaggtaacggcaacccttggtaacctaacccaaaatagggaagtggtctacgactcgcaaagtcatcactgccacgttgacaaggtcgagaaggaagttccagattatgagatgtggatgctcgatgcgggagggcttgaagtggaagtcgagtgctgccgtcaaaagcttgaagagttggcgtctggcaggaaccaaatgtatccccatctcaaggactgctag

SEQ ID NO: 14 - protein, 145 aa, Ornithodoros moubata
CTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 15 - protein, 20 aa, Artificial Sequence
  PAS sequence
ASPAAPAPASPAAPAPSAPA

SEQ ID NO: 16 - protein, 20 aa, Artificial Sequence
  PAS sequence
AAPASPAPAAPSAPAPAAPS

SEQ ID NO: 17 - protein, 20 aa, Artificial Sequence
  PAS sequence
APSSPSPSAPSSPSPASPSS

SEQ ID NO: 18 - protein, 20 aa, Artificial Sequence
  PAS sequence
SAPSSPSPSAPSSPSPASPS

SEQ ID NO: 19 - protein, 20 aa, Artificial Sequence
  PAS sequence
SSPSAPSPSSPASPSPSSPA

SEQ ID NO: 20 - protein, 24 aa, Artificial Sequence
  PAS sequence
AASPAAPSAPPAAASPAAPSAPPA

SEQ ID NO: 21 - protein, 20 aa, Artificial Sequence
  PAS sequence
ASAAAPAAASAAASAPSAAA

SEQ ID NO: 22 - protein, 150 aa, Artificial Sequence
  nomacopan variant 1
DSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEQWQSNGSADDKEVECCRQKLEELASGRNQMYPHLKDC

SEQ ID NO: 23 - protein, 150 aa, Artificial Sequence
  nomacopan variant 2
DSESDCTGSEPVDAFQAFSEGKEAYVLVRSTDPKARDCLKGEPNGEKQDNTLPVMMTFKNGTDWASTDWTFTLDGAKVTATLGNLTQNREVVYDSQSHHCHVDKVEKEVPDYEMWQSDAGADAVEVECCRQKLEELASGRNQMYPHLKGC

SEQ ID NO: 24 - protein, 150 aa, Artificial Sequence
  nomacopan variant 3
DSESDCTGSEPVDAFQAFSEGKEAYVLVRSTDPKARDCLKGEPNGEKQDNTLPVMMTFKNGTDWASTDWTFTLDGAKVTATLGNLTQNREVVYDSQSHHCHVDKVEKEVPDYEMWQLDAGGDEVEVECCRQKLEELASGRNQMYPHLKGC

SEQ ID NO: 25 - protein, 150 aa, Artificial Sequence
  nomacopan variant 4
DSESDCTGSEPVDAFQAFSEGKEAYVLVRSTDPKARDCLKGEPNGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 26 - protein, 11 aa, Artificial Sequence
  Loop sequence
MWMLDAGGLEV

SEQ ID NO: 27 - protein, 11 aa, Artificial Sequence
  Loop sequence of nomacopan variant 1
QWQSNGSADDK

SEQ ID NO: 28 - protein, 11 aa, Artificial Sequence
  Loop sequence of nomacopan variant 2
MWQSDAGADAV

SEQ ID NO: 29 - protein, 11 aa, Artificial Sequence
  Loop sequence of nomacopan variant 3
MWQLDAGGDEV

SEQ ID NO: 30 - protein, 751 aa, Artificial Sequence
  PAS-nomacopan
ASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAADSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDC

SEQ ID NO: 31 - protein, 751 aa, Artificial Sequence
  PAS-L-nomacopan
ASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAADSESDCTGSEPVDAFQAFSEGKEAYVLVRSTDPKARDCLKGEPNGEKQDNTLPVMMTFKNGTDWASTDWTFTLDGAKVTATLGNLTQNREVVYDSQSHHCHVDKVEKEVPDYEMWQSDAGADAVEVECCRQKLEELASGRNQMYPHLKGC

SEQ ID NO: 32 - protein, 20 aa, Artificial Sequence
  interphotoreceptor retinoid-binding protein (IRBP)
SGIPYIISYLHPGNTILHVD